SEQUENCE LISTING (389 sequences)

SEQ ID NO: 1 - RNA, 20 nt, synthetic construct
  misc_feature 1..20: note = Synthetic RNA
  source 1..20: mol_type = other RNA
ctgttgcctccggttctgaa

SEQ ID NO: 2 - protein, 1368 aa, synthetic construct [sequence omitted]

SEQ ID NO: 3 - DNA, 36 bp, synthetic construct
  misc_feature 1..36: note = Synthetic DNA
  source 1..36: mol_type = other DNA
aagaacaccttcagaaccggaggcaacagttgaatg

SEQ ID NO: 4 - DNA, 36 bp, synthetic construct
  misc_feature 1..36: note = Synthetic DNA
  source 1..36: mol_type = other DNA
cattcaactgttgcctccggttctgaaggtgttctt

SEQ ID NO: 5 - protein, 5 aa, synthetic construct
  REGION 1..5: note = Synthetic peptide
LRVQR

SEQ ID NO: 6 - protein, 1368 aa, synthetic construct
  REGION 1..1368: note = Synthetic protein
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLSPTVAYSVLVVAKVEKGKSKKLKSVKELLGITIMERSSFEKNPIDFLEAKGYKEVKKDLIIKLPKYSLFELENGRKRMLASARVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 7 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 8 - protein, 11 aa, synthetic construct
  REGION 1..11: note = Synthetic amino acid
MLWWEEVEDCY

SEQ ID NO: 9 - protein, 7 aa, synthetic construct
  REGION 1..7: note = Synthetic amino acid
MSEVSSD

SEQ ID NO: 10 - protein, 16 aa, synthetic construct
  REGION 1..16: note = Synthetic amino acid
MTEIILLIFFPAYFLN

SEQ ID NO: 11 - protein, 13 aa, synthetic construct
  REGION 1..13: note = Synthetic amino acid
MSARKLRNLSYKK

SEQ ID NO: 12 - protein, 10 aa, synthetic construct
  REGION 1..10: note = Synthetic amino acid
MLHRKTYHVK

SEQ ID NO: 13 - DNA, 20 bp, synthetic construct
  misc_feature 1..20: note = Synthetic oligonucleotide
  source 1..20: mol_type = other DNA
ctgttgcctccggttctgaa

SEQ ID NO: 14 - protein, 5 aa, synthetic construct
  REGION 1..5: note = Synthetic peptide
LKVQR

SEQ ID NO: 15 - protein, 5 aa, synthetic construct
  REGION 1..5: note = Synthetic peptide
LSVQR

SEQ ID NO: 16 - protein, 6 aa, synthetic construct
  REGION 1..6: note = Synthetic peptide
LWKVQR

SEQ ID NO: 17 - protein, 6 aa, synthetic construct
  REGION 1..6: note = Synthetic peptide
LWSVQR

SEQ ID NO: 18 - protein, 6 aa, synthetic construct
  REGION 1..6: note = Synthetic peptide
LWRVQR

SEQ ID NO: 19 - protein, 6 aa, synthetic construct
  REGION 1..6: note = Synthetic peptide
LWKSQR

SEQ ID NO: 20 - protein, 6 aa, synthetic construct
  REGION 1..6: note = Synthetic peptide
LWSSQR

SEQ ID NO: 21 - protein, 6 aa, synthetic construct
  REGION 1..6: note = Synthetic peptide
LWRSQR

SEQ ID NO: 22 - protein, 5 aa, synthetic construct
  REGION 1..5: note = Synthetic peptide
LKVQR

SEQ ID NO: 23 - protein, 5 aa, synthetic construct
  REGION 1..5: note = Synthetic peptide
LSVQR

SEQ ID NO: 24 - protein, 6 aa, synthetic construct
  REGION 1..6: note = Synthetic peptide
LWKVQR

SEQ ID NO: 25 - protein, 6 aa, synthetic construct
  REGION 1..6: note = Synthetic peptide
LWSVQR

SEQ ID NO: 26 - protein, 6 aa, synthetic construct
  REGION 1..6: note = Synthetic peptide
LWRVQR

SEQ ID NO: 27 - protein, 6 aa, synthetic construct
  REGION 1..6: note = Synthetic peptide
LWKSQR

SEQ ID NO: 28 - protein, 6 aa, synthetic construct
  REGION 1..6: note = Synthetic peptide
LWSSQR

SEQ ID NO: 29 - protein, 6 aa, synthetic construct
  REGION 1..6: note = Synthetic peptide
LWRSQR

SEQ ID NO: 30 - DNA, 450 bp, synthetic construct
  misc_feature 1..450: note = Synthetic nucleic acid
  source 1..450: mol_type = other DNA
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 31 - DNA, 436 bp, synthetic construct
  misc_feature 1..436: note = Synthetic nucleic acid
  source 1..436: mol_type = other DNA
tgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 32 - protein, 18 aa, Thosea asigna virus
EGRGSLLTCGDVEENPGP

SEQ ID NO: 33 - protein, 20 aa, synthetic construct
  REGION 1..20: note = Synthetic amino acid
QCTNYALLKLAGDVESNPGP

SEQ ID NO: 34 - protein, 19 aa, synthetic construct
  REGION 1..19: note = Synthetic amino acid
ATNFSLLKQAGDVEENPGP

SEQ ID NO: 35 - protein, 23 aa, synthetic construct
  REGION 1..23: note = Synthetic amino acid
PVKQLLNFDLLKLAGDVESNPGP

SEQ ID NO: 36 - protein, 41 aa, synthetic construct
  REGION 1..41: note = Synthetic amino acid
RMRKFKNKGKDTAELRRRRVEVSVELRKAKKDEQILKRRNV

SEQ ID NO: 37 - protein, 8 aa, synthetic construct
  REGION 1..8: note = Synthetic amino acid
VSRKRPRP

SEQ ID NO: 38 - protein, 8 aa, synthetic construct
  REGION 1..8: note = Synthetic amino acid
PPKKARED

SEQ ID NO: 39 - protein, 8 aa, synthetic construct
  REGION 1..8: note = Synthetic amino acid
PQPKKKPL

SEQ ID NO: 40 - protein, 12 aa, synthetic construct
  REGION 1..12: note = Synthetic amino acid
SALIKKKKKMAP

SEQ ID NO: 41 - protein, 5 aa, synthetic construct
  REGION 1..5: note = Synthetic amino acid
DRLRR

SEQ ID NO: 42 - protein, 6 aa, synthetic construct
  REGION 1..6: note = Synthetic amino acid
KQKKRK

SEQ ID NO: 43 - protein, 10 aa, synthetic construct
  REGION 1..10: note = Synthetic amino acid
RKLKKKIKKL

SEQ ID NO: 44 - protein, 10 aa, synthetic construct
  REGION 1..10: note = Synthetic amino acid
REKKKFLKRR

SEQ ID NO: 45 - protein, 20 aa, synthetic construct
  REGION 1..20: note = Synthetic amino acid
KRKGDEVDGVDEVAKKKSKK

SEQ ID NO: 46 - protein, 17 aa, synthetic construct
  REGION 1..17: note = Synthetic amino acid
RKCLQAGMNLEARKTKK

SEQ ID NO: 47 - DNA, 115 bp, synthetic construct
  misc_feature 1..115: note = Synthetic nucleotide
  source 1..115: mol_type = other DNA
ggccactccctctctgcgcgctcgctcgctcactgaggccgggcgaccaaaggtcgcccgacgcccgggctttgcccgggcggcctcagtgagcgagcgagcgcgcagagaggga

SEQ ID NO: 48 - DNA, 141 bp, synthetic construct
  misc_feature 1..141: note = Synthetic nucleotide
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 49 - DNA, 141 bp, synthetic construct
  misc_feature 1..141: note = Synthetic nucleotide
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 50 - DNA, 60 bp, synthetic construct
  misc_feature 1..60: note = Synthetic nucleotide
  source 1..60: mol_type = other DNA
tagcaataaaggatcgtttattttcattggaagcgtgtgttggttttttgatcaggcgcg

SEQ ID NO: 51 - DNA, 48 bp, synthetic construct
  misc_feature 1..48: note = Synthetic nucleotide
  source 1..48: mol_type = other DNA
aagcgtcctgctgctactaagaaagctggtcaagctaagaaaaagaaa

SEQ ID NO: 52 - DNA, 51 bp, synthetic construct
  misc_feature 1..51: note = Synthetic nucleotide
  source 1..51: mol_type = other DNA
atggccccaaagaagaagcggaaggtcggtatccacggagtcccagcagcc

SEQ ID NO: 53 - DNA, 166 bp, synthetic construct
  misc_feature 1..166: note = Synthetic nucleotide
  source 1..166: mol_type = other DNA
tgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggtt

SEQ ID NO: 54 - DNA, 225 bp, synthetic construct
  misc_feature 1..225: note = Synthetic nucleotide
  source 1..225: mol_type = other DNA
gaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccaca

SEQ ID NO: 55 - DNA, 1155 bp, synthetic construct
  misc_feature 1..1155: note = Synthetic nucleotide
  source 1..1155: mol_type = other DNA
catgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccg

SEQ ID NO: 56 - DNA, 472 bp, synthetic construct
  misc_feature 1..472: note = Synthetic nucleotide
  source 1..472: mol_type = other DNA
cgagtccaacacccgtgggaatcccatgggcaccatggcccctcgctccaaaaatgctttcgcgtcgcgcagacactgctcggtagtttcggggatcagcgtttgagtaagagcccgcgtctgaaccctccgcgccgccccggccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaa

SEQ ID NO: 57 - DNA, 108 bp, synthetic construct
  misc_feature 1..108: note = Synthetic nucleotide
  source 1..108: mol_type = other DNA
gctcggcgcgcccatatttgcatgtcgctatgtgttctgggaaatcaccataaacgtgaaatgtctttggatttgggaatcttataagttctgtatgagaccacggta

SEQ ID NO: 58 - DNA, 251 bp, synthetic construct
  misc_feature 1..251: note = Synthetic nucleotide
  source 1..251: mol_type = other DNA
tgacggcgcgccctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggta

SEQ ID NO: 59 - DNA, 141 bp, synthetic construct; identical to SEQ ID NO: 48
  misc_feature 1..141: note = Synthetic nucleotide
  source 1..141: mol_type = other DNA

SEQ ID NO: 60 - DNA, 141 bp, synthetic construct
  misc_feature 1..141: note = Synthetic nucleotide
  source 1..141: mol_type = other DNA
ccactccctctatgcgcgctcgctcactcactcggccctggagaccaaaggtctccagactgccggcctctggccggcagggccgagtgagtgagcgagcgcgcatagagggagtgggtacctccatcatctaggtttgcc

SEQ ID NO: 61 - DNA, 141 bp, synthetic construct
  misc_feature 1..141: note = Synthetic nucleotide
  source 1..141: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtgg

SEQ ID NO: 62 - DNA, 141 bp, synthetic construct; identical to SEQ ID NO: 49
  misc_feature 1..141: note = Synthetic nucleotide
  source 1..141: mol_type = other DNA

SEQ ID NO: 63 - DNA, 60 bp, synthetic construct; identical to SEQ ID NO: 50
  misc_feature 1..60: note = Synthetic nucleotide
  source 1..60: mol_type = other DNA

SEQ ID NO: 64 - DNA, 166 bp, synthetic construct; identical to SEQ ID NO: 53
  misc_feature 1..166: note = Synthetic nucleotide
  source 1..166: mol_type = other DNA

SEQ ID NO: 65 - DNA, 225 bp, synthetic construct; identical to SEQ ID NO: 54
  misc_feature 1..225: note = Synthetic nucleotide
  source 1..225: mol_type = other DNA

SEQ ID NO: 66 - DNA, 1155 bp, synthetic construct; identical to SEQ ID NO: 55
  misc_feature 1..1155: note = Synthetic nucleotide
  source 1..1155: mol_type = other DNA

SEQ ID NO: 67 - protein, 2158 aa, Mus musculus
MWWVDCYRDVKKTTKWNASKGKHDNSDDGKRDGTGKKKGSTRVHANNVNKARVKNNVDVNGSTDVDGNHKTGWNHWVKNVMKTMAGTNSKSWVRSTRNYVNVNTSSWSDGANAHSHRDDWNSVVSHSATRHANAKCGKDDVATTYDKKSMYTSVVSAVMRTSSKVTRHHHMHYSTVSAGYTSSSKRKSYATAAYVATSDSTSYSHARDKSDSSMTVNDSYTAVSWSADTRAGSNDVVKHAHGMMDTSHGVGNVGSVGKGKSDAVMNNSRWCRVASMKSKHKVMDNKKDDWTKTRTKKMGDDKCVHKVDVRVNSTHMVVVVDSSGDHATAAKVGDRWANCRWTDRWVDKWHTCSTWSKDAMKNTSGKDNMMSSHKSTKDKKKTMKSSNDSAKNKSVTKMWMNARWDNTKKSSASAVTTTSTTTVMTVTMVTTRMVKHAKKRTVDSRKRDVDTHSWTRSAVSSAVYRKGNSDKVNAARKAKRKDASRSAAVMANGVNASRASNSRWTCSRVNWYTNTYNMTTTANKTSTTSTAKSKCKDVNRSAKSKKGGMDADVATNHNHDGVRAKKTDTMRYTMSSRTWSSKSVYSVTYMRGKASSKNGNYSDTVKMAKKASCKYSGHWKKSSVSCKHMNKRKNHKTKWMAVDVKWAGDAKKKCRVGDTSNSVNGGKKSAASRTRNTWDHCRVYTRKAKAGDKTVSKDSMHWMTAYRDYKTDTAVMKRAKAKTKVKTTVNSVAHASAAKKTTTNYWCTRNGKCKTVWACWHSYKANKWNVKKTMNVAGTVSNMHHSNNRATTDGGVMDNTNSRWRHAVRKKSSAKSHSDKAAYTDKVDAAMAKSDTSHSMKKHNGKDANRVSDVAKKDVSMKRKANRSKMDVKMHATKSVVSSHCVNYKSSVKSVMVKTGRVKKTNKDRVTAKHYNGAKVTRKKCKSRKMRKMNVTWAATDTTKRSAVGMSNDSVAWGKATKKKAHKSVTGSKMVGKKTVDKSNSNWAVTSRVWNYKHMTDNTKWHADDSKKKKDKRKAMNDMRKVDSTRDAAKMANRGDHCRKVVSNRRAASHRKTGKASKNSDKAGVNKDNKDMSDNGTVNRGDNRTDRKRKKTKHNAKDRSRRKKASHWYYKRADDKCDKKASRDRKKDRKKKNAVRRAGSNGAAMAVTSKRWRSNARRNAHTHTMVVTTDMDVSYVSTYTSHASVDHNTCAKDDKSKNKDNSGRDHKKKTAASATSMKVKVAVAMDGKHRMYKRGRDRSVKWRHHYDMKVNWNVKKTNNWHAKYKWYKDGGRAVVRTNATGSSKTDVNKGSSRWHDCKARRKRKNVSRDNVWADNATGDKVKARGKNTGGAVVSARDKKKKTNWKVSRAKGVHKDRDHWSRNYNSAGDKVTVHGKADVRSKGHYKKSTVKRKDRSWAVNHRRTKDRAGSTTGASASTVTVTSVVTKTVSKMSSVAADNRAWTTDWSDRVKSRVMVGDDNMKKATDRRTAANKNKTSNARTTDRRWDVNRRNMKDSTWAKAVGVRGKDSWKGHTVDAKKTTKAKDRRSVDVANDAKRDYSADDTRKVHMTNNTSWGNHKRVSAATHRDKSWTATTANVDASRKKDSRGVRMKWDGTHTDYHNDNGKRSGSDARRDNMNKWSKKSNRSHASSDWKRHSVWKDDSRAGGDAVKNDHRAKRKTKVMSTTVRTGKYRRANVTRRKAVNAWDKNRSADWRKDARAADDKRAVKGSWVGDDSDHKVKARGAKNVNRVNDAHTTGSYNSTDNTRWRVAVDRVRHAHRDGASHSTSVGWRASNKVYYNHTTTCWDHKMTYSADNNVRSAYRTAMKRRKACDSSAACDADHNKNDMDNCTTYDRHNNVNVCVDMCNWNVYDTGRTGRRVSKTGSCKAHDKYRYKVASSTGCDRRGHDSRGVASGGSNSVRSCANNKAADWMRSMVWVHRVAAATAKHAKCNCKCGRYRSKHNYDCSCSGRVAKGHKMHYMVYCTTTSGDVRDAKVKNKRTKRYAKHRMGYVTVGDNMTVTNWVDSAASSSHDDTHSRHYASRAMNSNGSYNDSSNSDDHHYCSNDSSRSASSRGRADNRNAYDRKHHKGSSMMTSSRDAAAKRHKGRARMDHNKSHRRAAKVNGTTVSSSTSRSDSSMRVVGSTSSMGDSDTSTGVMNNSSSRGRNAGKMRDTM

SEQ ID NO: 68 - DNA, 20 bp, synthetic construct
  misc_feature 1..20: note = Synthetic oligonucleotide
  source 1..20: mol_type = other DNA
tagtggtcagtccaggagct

SEQ ID NO: 69 - DNA, 20 bp, synthetic construct
  misc_feature 1..20: note = Synthetic oligonucleotide
  source 1..20: mol_type = other DNA
gctccaatagtggtcagtcc

SEQ ID NO: 70 - DNA, 20 bp, synthetic construct
  misc_feature 1..20: note = Synthetic oligonucleotide
  source 1..20: mol_type = other DNA
gctcctggactgaccactat

SEQ ID NO: 71 - DNA, 20 bp, synthetic construct
  misc_feature 1..20: note = Synthetic oligonucleotide
  source 1..20: mol_type = other DNA
atacttacaggctccaatag

SEQ ID NO: 72 - DNA, 20 bp, synthetic construct
  misc_feature 1..20: note = Synthetic oligonucleotide
  source 1..20: mol_type = other DNA
atgggatccagtatacttac

SEQ ID NO: 73 - DNA, 20 bp, synthetic construct
  misc_feature 1..20: note = Synthetic oligonucleotide
  source 1..20: mol_type = other DNA
attggagcctgtaagtatac

SEQ ID NO: 74 - DNA, 20 bp, synthetic construct
  misc_feature 1..20: note = Synthetic oligonucleotide
  source 1..20: mol_type = other DNA
cagagtaacagtctgagtag

SEQ ID NO: 75 - DNA, 20 bp, synthetic construct
  misc_feature 1..20: note = Synthetic oligonucleotide
  source 1..20: mol_type = other DNA
caccagagtaacagtctgag

SEQ ID NO: 76 - DNA, 20 bp, synthetic construct
  misc_feature 1..20: note = Synthetic oligonucleotide
  source 1..20: mol_type = other DNA
tattttgggtttttgcaaaa

SEQ ID NO: 77 - DNA, 20 bp, synthetic construct
  misc_feature 1..20: note = Synthetic oligonucleotide
  source 1..20: mol_type = other DNA
agtaggagctaaaatatttt

SEQ ID NO: 78 - DNA, 20 bp, synthetic construct
  misc_feature 1..20: note = Synthetic oligonucleotide
  source 1..20: mol_type = other DNA
gagtaggagctaaaatattt

SEQ ID NO: 79 - DNA, 20 bp, synthetic construct
  misc_feature 1..20: note = Synthetic oligonucleotide
  source 1..20: mol_type = other DNA
accagagtaacagtctgagt

SEQ ID NO: 80 - DNA, 20 bp, synthetic construct
  misc_feature 1..20: note = Synthetic oligonucleotide
  source 1..20: mol_type = other DNA
tcctactcagactgttactc

SEQ ID NO: 81 - DNA, 20 bp, synthetic construct
  misc_feature 1..20: note = Synthetic oligonucleotide
  source 1..20: mol_type = other DNA
tactctggtgacacaacctg

SEQ ID NO: 82 - DNA, 20 bp, synthetic construct
  misc_feature 1..20: note = Synthetic oligonucleotide
  source 1..20: mol_type = other DNA
gcagtttccttagtaaccac

SEQ ID NO: 83 - DNA, 20 bp, synthetic construct
  misc_feature 1..20: note = Synthetic oligonucleotide
  source 1..20: mol_type = other DNA
gacacaacctgtggttacta

SEQ ID NO: 84 - DNA, 20 bp, synthetic construct
  misc_feature 1..20: note = Synthetic oligonucleotide
  source 1..20: mol_type = other DNA
tgtcaccagagtaacagtct

SEQ ID NO: 85 - DNA, 20 bp, synthetic construct
  misc_feature 1..20: note = Synthetic oligonucleotide
  source 1..20: mol_type = other DNA
aggttgtgtcaccagagtaa

SEQ ID NO: 86 - DNA, 20 bp, synthetic construct
  misc_feature 1..20: note = Synthetic oligonucleotide
  source 1..20: mol_type = other DNA
aaccacaggttgtgtcacca

SEQ ID NO: 87 - DNA, 20 bp, synthetic construct
  misc_feature 1..20: note = Synthetic oligonucleotide
  source 1..20: mol_type = other DNA
gtaaccacaggttgtgtcac

SEQ ID NO: 88 - DNA, 20 bp, synthetic construct
  misc_feature 1..20: note = Synthetic oligonucleotide
  source 1..20: mol_type = other DNA
atttatttttccttttattc

SEQ ID NO: 89 - DNA, 20 bp, synthetic construct
  misc_feature 1..20: note = Synthetic oligonucleotide
  source 1..20: mol_type = other DNA
tttccttttattctagttga

SEQ ID NO: 90 - DNA, 20 bp, synthetic construct
  misc_feature 1..20: note = Synthetic oligonucleotide
  source 1..20: mol_type = other DNA
tgattctgaattctttcaac

SEQ ID NO: 91 - DNA, 20 bp, synthetic construct
  misc_feature 1..20: note = Synthetic oligonucleotide
  source 1..20: mol_type = other DNA
aattctttcaactagaataa

SEQ ID NO: 92 - DNA, 20 bp, synthetic construct
  misc_feature 1..20: note = Synthetic oligonucleotide
  source 1..20: mol_type = other DNA
ttattctagttgaaagaatt

SEQ ID NO: 93 - DNA, 20 bp, synthetic construct
  misc_feature 1..20: note = Synthetic oligonucleotide
  source 1..20: mol_type = other DNA
tagttgaaagaattcagaat

SEQ ID NO: 94 - DNA, 20 bp, synthetic construct
  misc_feature 1..20: note = Synthetic oligonucleotide
  source 1..20: mol_type = other DNA
aattcagaatcagtgggatg

SEQ ID NO: 95 - DNA, 20 bp, synthetic construct
  misc_feature 1..20: note = Synthetic oligonucleotide
  source 1..20: mol_type = other DNA
attctttcaactagaataaa

SEQ ID NO: 96 - DNA, 20 bp, synthetic construct
  misc_feature 1..20: note = Synthetic oligonucleotide
  source 1..20: mol_type = other DNA
ttgaaagaattcagaatcag

SEQ ID NO: 97 - DNA, 20 bp, synthetic construct
  misc_feature 1..20: note = Synthetic oligonucleotide
  source 1..20: mol_type = other DNA
tgaaagaattcagaatcagt

SEQ ID NO: 98 - DNA, 20 bp, synthetic construct
  misc_feature 1..20: note = Synthetic oligonucleotide
  source 1..20: mol_type = other DNA
actgttgcctccggttctga

SEQ ID NO: 99 - DNA, 20 bp, synthetic construct
  misc_feature 1..20: note = Synthetic oligonucleotide
  source 1..20: mol_type = other DNA
cagatctgtcaaatcgcctg

SEQ ID NO: 100 - DNA, 20 bp, synthetic construct
  misc_feature 1..20: note = Synthetic oligonucleotide
  source 1..20: mol_type = other DNA
aaaacgccgccatttctcaa

SEQ ID NO: 101 - DNA, 20 bp, synthetic construct
  misc_feature 1..20: note = Synthetic oligonucleotide
  source 1..20: mol_type = other DNA
agatctgtcaaatcgcctgc

SEQ ID NO: 102 - DNA, 20 bp, synthetic construct
  misc_feature 1..20: note = Synthetic oligonucleotide
  source 1..20: mol_type = other DNA
tatggatcaagaaaaataga

SEQ ID NO: 103 - DNA, 20 bp, synthetic construct
  misc_feature 1..20: note = Synthetic oligonucleotide
  source 1..20: mol_type = other DNA
cgcctgcaggtaaaagcata

SEQ ID NO: 104 - DNA, 20 bp, synthetic construct
  misc_feature 1..20: note = Synthetic oligonucleotide
  source 1..20: mol_type = other DNA
atccatatgcttttacctgc

SEQ ID NO: 105 - DNA, 20 bp, synthetic construct
  misc_feature 1..20: note = Synthetic oligonucleotide
  source 1..20: mol_type = other DNA
ttgacagatctgttgagaaa

SEQ ID NO: 106 - DNA, 20 bp, synthetic construct
  misc_feature 1..20: note = Synthetic oligonucleotide
  source 1..20: mol_type = other DNA
acagatctgttgagaaatgg

SEQ ID NO: 107 - DNA, 20 bp, synthetic construct
  misc_feature 1..20: note = Synthetic oligonucleotide
  source 1..20: mol_type = other DNA
ggcgatttgacagatctgtt

SEQ ID NO: 108 - DNA, 20 bp, synthetic construct
  misc_feature 1..20: note = Synthetic oligonucleotide
  source 1..20: mol_type = other DNA
ggcgttttcattatgatata

SEQ ID NO: 109 - DNA, 20 bp, synthetic construct
  misc_feature 1..20: note = Synthetic oligonucleotide
  source 1..20: mol_type = other DNA
atgatataaagatatttaat

SEQ ID NO: 110 - DNA, 20 bp, synthetic construct
  misc_feature 1..20: note = Synthetic oligonucleotide
  source 1..20: mol_type = other DNA
gatatttaatcagtggctaa

SEQ ID NO: 111 - DNA, 20 bp, synthetic construct
  misc_feature 1..20: note = Synthetic oligonucleotide
  source 1..20: mol_type = other DNA
atttaatcagtggctaacag

SEQ ID NO: 112 - DNA, 20 bp, synthetic construct
  misc_feature 1..20: note = Synthetic oligonucleotide
  source 1..20: mol_type = other DNA
agaaactgttcagcttctgt

SEQ ID NO: 113 - DNA, 20 bp, synthetic construct
  misc_feature 1..20: note = Synthetic oligonucleotide
  source 1..20: mol_type = other DNA
gggaacatgctaaatacaaa

SEQ ID NO: 114 - DNA, 20 bp, synthetic construct
  misc_feature 1..20: note = Synthetic oligonucleotide
  source 1..20: mol_type = other DNA
taaatacaaatggtatctta

SEQ ID NO: 115 - DNA, 20 bp, synthetic construct
  misc_feature 1..20: note = Synthetic oligonucleotide
  source 1..20: mol_type = other DNA
gttttaaaatttttatatta

SEQ ID NO: 116 - DNA, 20 bp, synthetic construct
  misc_feature 1..20: note = Synthetic oligonucleotide
  source 1..20: mol_type = other DNA
ttttatattacagaatataa

SEQ ID NO: 117 - DNA, 20 bp, synthetic construct
  misc_feature 1..20: note = Synthetic oligonucleotide
  source 1..20: mol_type = other DNA
atattacagaatataaaaga

SEQ ID NO: 118 - DNA, 20 bp, synthetic construct
  misc_feature 1..20: note = Synthetic oligonucleotide
  source 1..20: mol_type = other DNA
gttcctgtaagataccaaaa

SEQ ID NO: 119 - DNA, 20 bp, synthetic construct
  misc_feature 1..20: note = Synthetic oligonucleotide
  source 1..20: mol_type = other DNA
ttgcctttttggtatcttac

SEQ ID NO: 120 - DNA, 20 bp, synthetic construct
  misc_feature 1..20: note = Synthetic oligonucleotide
  source 1..20: mol_type = other DNA
tggtatcttacaggaactcc

SEQ ID NO: 121 - DNA, 20 bp, synthetic construct
  misc_feature 1..20: note = Synthetic oligonucleotide
  source 1..20: mol_type = other DNA
atcttacaggaactccagga

SEQ ID NO: 122 - DNA, 20 bp, synthetic construct
  misc_feature 1..20: note = Synthetic oligonucleotide
  source 1..20: mol_type = other DNA
gccgctgcccaatgccatcc

SEQ ID NO: 123 - DNA, 20 bp, synthetic construct
  misc_feature 1..20: note = Synthetic oligonucleotide
  source 1..20: mol_type = other DNA
caggaactccaggatggcat

SEQ ID NO: 124 - DNA, 20 bp, synthetic construct
  misc_feature 1..20: note = Synthetic oligonucleotide
  source 1..20: mol_type = other DNA
aggaactccaggatggcatt

SEQ ID NO: 125 - DNA, 20 bp, synthetic construct
  misc_feature 1..20: note = Synthetic oligonucleotide
  source 1..20: mol_type = other DNA
tccaggatggcattgggcag

SEQ ID NO: 126 - DNA, 20 bp, synthetic construct
  misc_feature 1..20: note = Synthetic oligonucleotide
  source 1..20: mol_type = other DNA
gtcagaacattgaatgcaac

SEQ ID NO: 127 - DNA, 20 bp, synthetic construct
  misc_feature 1..20: note = Synthetic oligonucleotide
  source 1..20: mol_type = other DNA
agttcctgtaagataccaaa

SEQ ID NO: 128 - DNA, 20 bp, synthetic construct
  misc_feature 1..20: note = Synthetic oligonucleotide
  source 1..20: mol_type = other DNA
tgccatcctggagttcctgt

SEQ ID NO: 129 - DNA, 20 bp, synthetic construct
  misc_feature 1..20: note = Synthetic oligonucleotide
  source 1..20: mol_type = other DNA
ttggtatcttacaggaactc

SEQ ID NO: 130 - DNA, 20 bp, synthetic construct
  misc_feature 1..20: note = Synthetic oligonucleotide
  source 1..20: mol_type = other DNA
cgctgcccaatgccatcctg

SEQ ID NO: 131 - DNA, 20 bp, synthetic construct
  misc_feature 1..20: note = Synthetic oligonucleotide
  source 1..20: mol_type = other DNA
aactccaggatggcattggg

SEQ ID NO: 132 - DNA, 20 bp, synthetic construct
  misc_feature 1..20: note = Synthetic oligonucleotide
  source 1..20: mol_type = other DNA
gggcagcggcaaactgttgt

SEQ ID NO: 133 - DNA, 20 bp, synthetic construct
  misc_feature 1..20: note = Synthetic oligonucleotide
  source 1..20: mol_type = other DNA
attcttttgttcttctagcc

SEQ ID NO: 134 - DNA, 20 bp, synthetic construct
  misc_feature 1..20: note = Synthetic oligonucleotide
  source 1..20: mol_type = other DNA
tgctcttttccaggttcaag

SEQ ID NO: 135 - DNA, 20 bp, synthetic construct
  misc_feature 1..20: note = Synthetic oligonucleotide
  source 1..20: mol_type = other DNA
gctagtatcccacttgaacc

SEQ ID NO: 136 - DNA, 20 bp, synthetic construct
  misc_feature 1..20: note = Synthetic oligonucleotide
  source 1..20: mol_type = other DNA
tttagttgctgctcttttcc

SEQ ID NO: 137 - DNA, 20 bp, synthetic construct
  misc_feature 1..20: note = Synthetic oligonucleotide
  source 1..20: mol_type = other DNA
agaaaagcttgagcaagtca

SEQ ID NO: 138 - DNA, 20 bp, synthetic construct
  misc_feature 1..20: note = Synthetic oligonucleotide
  source 1..20: mol_type = other DNA
agatctgtcaaatcgcctgc

SEQ ID NO: 139 - DNA, 20 bp, synthetic construct
  misc_feature 1..20: note = Synthetic oligonucleotide
  source 1..20: mol_type = other DNA
aatcctgcattgttgcctgt

SEQ ID NO: 140 - DNA, 20 bp, synthetic construct
  misc_feature 1..20: note = Synthetic oligonucleotide
  source 1..20: mol_type = other DNA
cgctgaagaaccctgatact

SEQ ID NO: 141 - DNA, 20 bp, synthetic construct
  misc_feature 1..20: note = Synthetic oligonucleotide
  source 1..20: mol_type = other DNA
gaacaaatatcccttagtat

SEQ ID NO: 142 - DNA, 20 bp, synthetic construct
  misc_feature 1..20: note = Synthetic oligonucleotide
  source 1..20: mol_type = other DNA
ctgtaagaacaaatatccct

SEQ ID NO: 143 - DNA, 20 bp, synthetic construct
  misc_feature 1..20: note = Synthetic oligonucleotide
  source 1..20: mol_type = other DNA
ctaagggatatttgttctta

SEQ ID NO: 144 - DNA, 20 bp, synthetic construct
  misc_feature 1..20: note = Synthetic oligonucleotide
  source 1..20: mol_type = other DNA
tgttcttacaggcaacaatg

SEQ ID NO: 145 - DNA, 20 bp, synthetic construct
  misc_feature 1..20: note = Synthetic oligonucleotide
  source 1..20: mol_type = other DNA
caacaatgcaggatttggaa

SEQ ID NO: 146 - DNA, 20 bp, synthetic construct
  misc_feature 1..20: note = Synthetic oligonucleotide
  source 1..20: mol_type = other DNA
acaatgcaggatttggaaca

SEQ ID NO: 147 - DNA, 20 bp, synthetic construct
  misc_feature 1..20: note = Synthetic oligonucleotide
  source 1..20: mol_type = other DNA
atttggaacagaggcgtccc

SEQ ID NO: 148 - DNA, 20 bp, synthetic construct
  misc_feature 1..20: note = Synthetic oligonucleotide
  source 1..20: mol_type = other DNA
acagaggcgtccccagttgg

SEQ ID NO: 149 - DNA, 20 bp, synthetic construct
  misc_feature 1..20: note = Synthetic oligonucleotide
  source 1..20: mol_type = other DNA
tatttttttattttgcattt

SEQ ID NO: 150 - DNA, 20 bp, synthetic construct
  misc_feature 1..20: note = Synthetic oligonucleotide
  source 1..20: mol_type = other DNA
ttattttgcattttagatga

SEQ ID NO: 151 - DNA, 20 bp, synthetic construct
  misc_feature 1..20: note = Synthetic oligonucleotide
  source 1..20: mol_type = other DNA
attttgcattttagatgaaa

SEQ ID NO: 152 - DNA, 20 bp, synthetic construct
  misc_feature 1..20: note = Synthetic oligonucleotide
  source 1..20: mol_type = other DNA
ttgcattttagatgaaagag

SEQ ID NO: 153 - DNA, 20 bp, synthetic construct
  misc_feature 1..20: note = Synthetic oligonucleotide
  source 1..20: mol_type = other DNA
atgaaagagaagatgttcaa

SEQ ID NO: 154 - DNA, 19 bp, synthetic construct
  misc_feature 1..19: note = Synthetic oligonucleotide
  source 1..19: mol_type = other DNA
attggctttgatttcccta

SEQ ID NO: 155 - DNA, 19 bp, synthetic construct
  misc_feature 1..19: note = Synthetic oligonucleotide
  source 1..19: mol_type = other DNA
tgtagagtaagtcagccta

SEQ ID NO: 156 - DNA, 19 bp, synthetic construct
  misc_feature 1..19: note = Synthetic oligonucleotide
  source 1..19: mol_type = other DNA
cctactcagactgttactc

SEQ ID NO: 157 - DNA, 19 bp, synthetic construct
  misc_feature 1..19: note = Synthetic oligonucleotide
  source 1..19: mol_type = other DNA
ttggacagaacttaccgac

SEQ ID NO: 158 - DNA, 19 bp, synthetic construct
  misc_feature 1..19: note = Synthetic oligonucleotide
  source 1..19: mol_type = other DNA
cagttgcctaagaactggt

SEQ ID NO: 159 - DNA, 19 bp, synthetic construct
  misc_feature 1..19: note = Synthetic oligonucleotide
  source 1..19: mol_type = other DNA
gggctccaccctcacgagt

SEQ ID NO: 160 - DNA, 19 bp, synthetic construct
  misc_feature 1..19: note = Synthetic oligonucleotide
  source 1..19: mol_type = other DNA
tttgcttcgctataaaacg

SEQ ID NO: 161 - DNA, 19 bp, synthetic construct
  misc_feature 1..19: note = Synthetic oligonucleotide
  source 1..19: mol_type = other DNA
tctgaggatggggccgcaa

SEQ ID NO: 162 - DNA, 19 bp, synthetic construct
  misc_feature 1..19: note = Synthetic oligonucleotide
  source 1..19: mol_type = other DNA
gatctgtcaaatcgcctgc

SEQ ID NO: 163 - DNA, 19 bp, synthetic construct
  misc_feature 1..19: note = Synthetic oligonucleotide
  source 1..19: mol_type = other DNA
ccaggatggcattgggcag

SEQ ID NO: 164 - DNA, 19 bp, synthetic construct
  misc_feature 1..19: note = Synthetic oligonucleotide
  source 1..19: mol_type = other DNA
ctgaatctgcggtggcagg

SEQ ID NO: 165 - DNA, 19 bp, synthetic construct
  misc_feature 1..19: note = Synthetic oligonucleotide
  source 1..19: mol_type = other DNA
ttcttttgttcttctagcc

SEQ ID NO: 166 - DNA, 19 bp, synthetic construct
  misc_feature 1..19: note = Synthetic oligonucleotide
  source 1..19: mol_type = other DNA
gaaaagcttgagcaagtca

SEQ ID NO: 167 - DNA, 19 bp, synthetic construct
  misc_feature 1..19: note = Synthetic oligonucleotide
  source 1..19: mol_type = other DNA
gaagagttgcccctgcgcc

SEQ ID NO: 168 - DNA, 19 bp, synthetic construct
  misc_feature 1..19: note = Synthetic oligonucleotide
  source 1..19: mol_type = other DNA
acaaatctccagtggataa

SEQ ID NO: 169 - DNA, 19 bp, synthetic construct
  misc_feature 1..19: note = Synthetic oligonucleotide
  source 1..19: mol_type = other DNA
tgtttctcaggtaaagctc

SEQ ID NO: 170 - DNA, 19 bp, synthetic construct
  misc_feature 1..19: note = Synthetic oligonucleotide
  source 1..19: mol_type = other DNA
gaaggaccatttgacgtta

SEQ ID NO: 171 - DNA, 19 bp, synthetic construct
  misc_feature 1..19: note = Synthetic oligonucleotide
  source 1..19: mol_type = other DNA
aactgctatttcagtttcc

SEQ ID NO: 172 - DNA, 19 bp, synthetic construct
  misc_feature 1..19: note = Synthetic oligonucleotide
  source 1..19: mol_type = other DNA
ccagccactcagccagtga

SEQ ID NO: 173 - DNA, 19 bp, synthetic construct
  misc_feature 1..19: note = Synthetic oligonucleotide
  source 1..19: mol_type = other DNA
gtatgcttttctgttaaag

SEQ ID NO: 174 - DNA, 19 bp, synthetic construct
  misc_feature 1..19: note = Synthetic oligonucleotide
  source 1..19: mol_type = other DNA
ctcctggactgaccactat

SEQ ID NO: 175 - DNA, 19 bp, synthetic construct
  misc_feature 1..19: note = Synthetic oligonucleotide
  source 1..19: mol_type = other DNA
gaacagaggcgtccccagt

SEQ ID NO: 176 - DNA, 19 bp, synthetic construct
  misc_feature 1..19: note = Synthetic oligonucleotide
  source 1..19: mol_type = other DNA
gaggctagaacaatcatta

SEQ ID NO: 177 - DNA, 19 bp, synthetic construct
  misc_feature 1..19: note = Synthetic oligonucleotide
  source 1..19: mol_type = other DNA
acaagaacaccttcagaac

SEQ ID NO: 178 - DNA, 19 bp, synthetic construct
  misc_feature 1..19: note = Synthetic oligonucleotide
  source 1..19: mol_type = other DNA
ggtttctgtgattttcttt

SEQ ID NO: 179 - DNA, 19 bp, synthetic construct
  misc_feature 1..19: note = Synthetic oligonucleotide
  source 1..19: mol_type = other DNA
ggccaaagacctccgccag

SEQ ID NO: 180 - DNA, 19 bp, synthetic construct
  misc_feature 1..19: note = Synthetic oligonucleotide
  source 1..19: mol_type = other DNA
ttggagaagcattcataaa

SEQ ID NO: 181 - DNA, 19 bp, synthetic construct
  misc_feature 1..19: note = Synthetic oligonucleotide
  source 1..19: mol_type = other DNA
tcgctcactcaccctgcaa

SEQ ID NO: 182 - DNA, 19 bp, synthetic construct
  misc_feature 1..19: note = Synthetic oligonucleotide
  source 1..19: mol_type = other DNA
aaaagagctgatgaaacaa

SEQ ID NO: 183 - DNA, 19 bp, synthetic construct
  misc_feature 1..19: note = Synthetic oligonucleotide
  source 1..19: mol_type = other DNA
tacacttttcaaaatgctt

SEQ ID NO: 184 - DNA, 19 bp, synthetic construct
  misc_feature 1..19: note = Synthetic oligonucleotide
  source 1..19: mol_type = other DNA
gagatgatcatcaagcaga

SEQ ID NO: 185 - DNA, 19 bp, synthetic construct
  misc_feature 1..19: note = Synthetic oligonucleotide
  source 1..19: mol_type = other DNA
ctttgaaagagcaataaaa

SEQ ID NO: 186 - DNA, 19 bp, synthetic construct
  misc_feature 1..19: note = Synthetic oligonucleotide
  source 1..19: mol_type = other DNA
cacaaaagtcaaatcggaa

SEQ ID NO: 187 - DNA, 19 bp, synthetic construct
  misc_feature 1..19: note = Synthetic oligonucleotide
  source 1..19: mol_type = other DNA
atttcaatataagattcgg

SEQ ID NO: 188 - DNA, 19 bp, synthetic construct
  misc_feature 1..19: note = Synthetic oligonucleotide
  source 1..19: mol_type = other DNA
cttaagcaatcccgaactc

SEQ ID NO: 189 - DNA, 19 bp, synthetic construct
  misc_feature 1..19: note = Synthetic oligonucleotide
  source 1..19: mol_type = other DNA
ccttctttatcccctatcg

SEQ ID NO: 190 - DNA, 19 bp, synthetic construct
  misc_feature 1..19: note = Synthetic oligonucleotide
  source 1..19: mol_type = other DNA
tagtgatcgtggatacgag

SEQ ID NO: 191 - DNA, 19 bp, synthetic construct
  misc_feature 1..19: note = Synthetic oligonucleotide
  source 1..19: mol_type = other DNA
tacagccctcggtgtatat

SEQ ID NO: 192 - DNA, 19 bp, synthetic construct
  misc_feature 1..19: note = Synthetic oligonucleotide
  source 1..19: mol_type = other DNA
ggaaggaattaagcccgaa

SEQ ID NO: 193 - DNA, 19 bp, synthetic construct
  misc_feature 1..19: note = Synthetic oligonucleotide
  source 1..19: mol_type = other DNA
ggaacagctttcgtagttg

SEQ ID NO: 194 - DNA, 19 bp, synthetic construct
  misc_feature 1..19: note = Synthetic oligonucleotide
  source 1..19: mol_type = other DNA
ataaagtccagtgtcgatc

SEQ ID NO: 195 - DNA, 19 bp, synthetic construct
  misc_feature 1..19: note = Synthetic oligonucleotide
  source 1..19: mol_type = other DNA
aaaaccagagcttcggtca

SEQ ID NO: 196 - DNA, 19 bp, synthetic construct
  misc_feature 1..19: note = Synthetic oligonucleotide
  source 1..19: mol_type = other DNA
attggctttgatttcccta

SEQ ID NO: 197 - DNA, 19 bp, synthetic construct
  misc_feature 1..19: note = Synthetic oligonucleotide
  source 1..19: mol_type = other DNA
tgtagagtaagtcagccta

SEQ ID NO: 198 - DNA, 19 bp, synthetic construct
  misc_feature 1..19: note = Synthetic oligonucleotide
  source 1..19: mol_type = other DNA
cctactcagactgttactc

SEQ ID NO: 199 - DNA, 19 bp, synthetic construct
  misc_feature 1..19: note = Synthetic oligonucleotide
  source 1..19: mol_type = other DNA
ttggacagaacttaccgac

SEQ ID NO: 200 - DNA, 19 bp, synthetic construct
  misc_feature 1..19: note = Synthetic oligonucleotide
  source 1..19: mol_type = other DNA
cagttgcctaagaactggt

SEQ ID NO: 201 - DNA, 19 bp, synthetic construct
  misc_feature 1..19: note = Synthetic oligonucleotide
  source 1..19: mol_type = other DNA
gggctccaccctcacgagt

SEQ ID NO: 202 - DNA, 19 bp, synthetic construct
  misc_feature 1..19: note = Synthetic oligonucleotide
  source 1..19: mol_type = other DNA
tttgcttcgctataaaacg

SEQ ID NO: 203 - DNA, 19 bp, synthetic construct
  misc_feature 1..19: note = Synthetic oligonucleotide
  source 1..19: mol_type = other DNA
tctgaggatggggccgcaa

SEQ ID NO: 204 - DNA, 19 bp, synthetic construct
  misc_feature 1..19: note = Synthetic oligonucleotide
  source 1..19: mol_type = other DNA
gatctgtcaaatcgcctgc

SEQ ID NO: 205 - DNA, 19 bp, synthetic construct
  misc_feature 1..19: note = Synthetic oligonucleotide
  source 1..19: mol_type = other DNA
ccaggatggcattgggcag

SEQ ID NO: 206 - DNA, 19 bp, synthetic construct
  misc_feature 1..19: note = Synthetic oligonucleotide
  source 1..19: mol_type = other DNA
ctgaatctgcggtggcagg

SEQ ID NO: 207 - DNA, 19 bp, synthetic construct
  misc_feature 1..19: note = Synthetic oligonucleotide
  source 1..19: mol_type = other DNA
ttcttttgttcttctagcc

SEQ ID NO: 208 - DNA, 19 bp, synthetic construct
  misc_feature 1..19: note = Synthetic oligonucleotide
  source 1..19: mol_type = other DNA
gaaaagcttgagcaagtca

SEQ ID NO: 209 - DNA, 19 bp, synthetic construct
  misc_feature 1..19: note = Synthetic oligonucleotide
  source 1..19: mol_type = other DNA
gaagagttgcccctgcgcc

SEQ ID NO: 210 - DNA, 19 bp, synthetic construct
  misc_feature 1..19: note = Synthetic oligonucleotide
  source 1..19: mol_type = other DNA
acaaatctccagtggataa

SEQ ID NO: 211 - DNA, 19 bp, synthetic construct
  misc_feature 1..19: note = Synthetic oligonucleotide
  source 1..19: mol_type = other DNA
tgtttctcaggtaaagctc

SEQ ID NO: 212 - DNA, 19 bp, synthetic construct
  misc_feature 1..19: note = Synthetic oligonucleotide
  source 1..19: mol_type = other DNA
gaaggaccatttgacgtta

SEQ ID NO: 213 - DNA, 19 bp, synthetic construct
  misc_feature 1..19: note = Synthetic oligonucleotide
  source 1..19: mol_type = other DNA
aactgctatttcagtttcc

SEQ ID NO: 214 - DNA, 19 bp, synthetic construct
  misc_feature 1..19: note = Synthetic oligonucleotide
  source 1..19: mol_type = other DNA
ccagccactcagccagtga

SEQ ID NO: 215 - DNA, 19 bp, synthetic construct
  misc_feature 1..19: note = Synthetic oligonucleotide
  source 1..19: mol_type = other DNA
gtatgcttttctgttaaag

SEQ ID NO: 216 - DNA, 19 bp, synthetic construct
  misc_feature 1..19: note = Synthetic oligonucleotide
  source 1..19: mol_type = other DNA
ctcctggactgaccactat

SEQ ID NO: 217 - DNA, 19 bp, synthetic construct
  misc_feature 1..19: note = Synthetic oligonucleotide
  source 1..19: mol_type = other DNA
gaacagaggcgtccccagt

SEQ ID NO: 218 - DNA, 19 bp, synthetic construct
  misc_feature 1..19: note = Synthetic oligonucleotide
  source 1..19: mol_type = other DNA
gaggctagaacaatcatta

SEQ ID NO: 219 - DNA, 19 bp, synthetic construct
  misc_feature 1..19: note = Synthetic oligonucleotide
  source 1..19: mol_type = other DNA
acaagaacaccttcagaac

SEQ ID NO: 220 - DNA, 19 bp, synthetic construct
  misc_feature 1..19: note = Synthetic oligonucleotide
  source 1..19: mol_type = other DNA
ggtttctgtgattttcttt

SEQ ID NO: 221 - DNA, 19 bp, synthetic construct
  misc_feature 1..19: note = Synthetic oligonucleotide
  source 1..19: mol_type = other DNA
ggccaaagacctccgccag

SEQ ID NO: 222 - DNA, 19 bp, synthetic construct
  misc_feature 1..19: note = Synthetic oligonucleotide
  source 1..19: mol_type = other DNA
ttggagaagcattcataaa

SEQ ID NO: 223 - DNA, 19 bp, synthetic construct
  misc_feature 1..19: note = Synthetic oligonucleotide
  source 1..19: mol_type = other DNA
tcgctcactcaccctgcaa

SEQ ID NO: 224 - DNA, 19 bp, synthetic construct
  misc_feature 1..19: note = Synthetic oligonucleotide
  source 1..19: mol_type = other DNA
aaaagagctgatgaaacaa

SEQ ID NO: 225 - DNA, 19 bp, synthetic construct
  misc_feature 1..19: note = Synthetic oligonucleotide
  source 1..19: mol_type = other DNA
tacacttttcaaaatgctt

SEQ ID NO: 226 - DNA, 19 bp, synthetic construct
  misc_feature 1..19: note = Synthetic oligonucleotide
  source 1..19: mol_type = other DNA
gagatgatcatcaagcaga

SEQ ID NO: 227 - DNA, 19 bp, synthetic construct
  misc_feature 1..19: note = Synthetic oligonucleotide
  source 1..19: mol_type = other DNA
cacaaaagtcaaatcggaa

SEQ ID NO: 228 - DNA, 19 bp, synthetic construct
  misc_feature 1..19: note = Synthetic oligonucleotide
  source 1..19: mol_type = other DNA
atttcaatataagattcgg

SEQ ID NO: 229 - DNA, 19 bp, synthetic construct
  misc_feature 1..19: note = Synthetic oligonucleotide
  source 1..19: mol_type = other DNA
cttaagcaatcccgaactc

SEQ ID NO: 230 - DNA, 19 bp, synthetic construct
  misc_feature 1..19: note = Synthetic oligonucleotide
  source 1..19: mol_type = other DNA
ccttctttatcccctatcg

SEQ ID NO: 231 - DNA, 19 bp, synthetic construct
  misc_feature 1..19: note = Synthetic oligonucleotide
  source 1..19: mol_type = other DNA
tagtgatcgtggatacgag

SEQ ID NO: 232 - DNA, 19 bp, synthetic construct
  misc_feature 1..19: note = Synthetic oligonucleotide
  source 1..19: mol_type = other DNA
tacagccctcggtgtatat

SEQ ID NO: 233 - DNA, 19 bp, synthetic construct
  misc_feature 1..19: note = Synthetic oligonucleotide
  source 1..19: mol_type = other DNA
ggaaggaattaagcccgaa

SEQ ID NO: 234 - DNA, 19 bp, synthetic construct
  misc_feature 1..19: note = Synthetic oligonucleotide
  source 1..19: mol_type = other DNA
ggaacagctttcgtagttg

SEQ ID NO: 235 - DNA, 19 bp, synthetic construct
  misc_feature 1..19: note = Synthetic oligonucleotide
  source 1..19: mol_type = other DNA
ataaagtccagtgtcgatc

SEQ ID NO: 236 - DNA, 19 bp, synthetic construct
  misc_feature 1..19: note = Synthetic oligonucleotide
  source 1..19: mol_type = other DNA
aaaaccagagcttcggtca

SEQ ID NO: 237 - DNA, 20 bp, synthetic construct
  misc_feature 1..20: note = Synthetic oligonucleotide
  source 1..20: mol_type = other DNA
gttgcctggtatgtctagcc

SEQ ID NO: 238 - DNA, 20 bp, synthetic construct
  misc_feature 1..20: note = Synthetic oligonucleotide
  source 1..20: mol_type = other DNA
aagaacaccttcagaaccgg

SEQ ID NO: 239 - DNA, 20 bp, synthetic construct
  misc_feature 1..20: note = Synthetic oligonucleotide
  source 1..20: mol_type = other DNA
ggtatgtctagcctgtcaca

SEQ ID NO: 240 - DNA, 20 bp, synthetic construct
  misc_feature 1..20: note = Synthetic oligonucleotide
  source 1..20: mol_type = other DNA
tgtgacaggctagacatacc

SEQ ID NO: 241 - DNA, 20 bp, synthetic construct
  misc_feature 1..20: note = Synthetic oligonucleotide
  source 1..20: mol_type = other DNA
gaggcatcagttgcctggta

SEQ ID NO: 242 - DNA, 20 bp, synthetic construct
  misc_feature 1..20: note = Synthetic oligonucleotide
  source 1..20: mol_type = other DNA
gttgcctccggttctgaagg

SEQ ID NO: 243 - DNA, 20 bp, synthetic construct
  misc_feature 1..20: note = Synthetic oligonucleotide
  source 1..20: mol_type = other DNA
agtagtaaatgctagtctgg

SEQ ID NO: 244 - DNA, 20 bp, synthetic construct
  misc_feature 1..20: note = Synthetic oligonucleotide
  source 1..20: mol_type = other DNA
tatagtagtaaatgctagtc

SEQ ID NO: 245 - DNA, 20 bp, synthetic construct
  misc_feature 1..20: note = Synthetic oligonucleotide
  source 1..20: mol_type = other DNA
tccggttctgaaggtgttct

SEQ ID NO: 246 - DNA, 20 bp, synthetic construct
  misc_feature 1..20: note = Synthetic oligonucleotide
  source 1..20: mol_type = other DNA
gaggggaggcatcagttgcc

SEQ ID NO: 247 - DNA, 20 bp, synthetic construct
  misc_feature 1..20: note = Synthetic oligonucleotide
  source 1..20: mol_type = other DNA
cataaatgtgagataacgtt

SEQ ID NO: 248 - DNA, 20 bp, synthetic construct
  misc_feature 1..20: note = Synthetic oligonucleotide
  source 1..20: mol_type = other DNA
gttctagggatcaagaattg

SEQ ID NO: 249 - DNA, 20 bp, synthetic construct
  misc_feature 1..20: note = Synthetic oligonucleotide
  source 1..20: mol_type = other DNA
aggggaggcatcagttgcct

SEQ ID NO: 250 - DNA, 20 bp, synthetic construct
  misc_feature 1..20: note = Synthetic oligonucleotide
  source 1..20: mol_type = other DNA
attatatacaggctgcctct

SEQ ID NO: 251 - DNA, 20 bp, synthetic construct
  misc_feature 1..20: note = Synthetic oligonucleotide
  source 1..20: mol_type = other DNA
aggggatatggatattctgc

SEQ ID NO: 252 - DNA, 20 bp, synthetic construct
  misc_feature 1..20: note = Synthetic oligonucleotide
  source 1..20: mol_type = other DNA
tgtctagcctgtcacatgga

SEQ ID NO: 253 - DNA, 20 bp, synthetic construct
  misc_feature 1..20: note = Synthetic oligonucleotide
  source 1..20: mol_type = other DNA
cattatatacaggctgcctc

SEQ ID NO: 254 - DNA, 20 bp, synthetic construct
  misc_feature 1..20: note = Synthetic oligonucleotide
  source 1..20: mol_type = other DNA
acccatcagcctgatttccc

SEQ ID NO: 255 - DNA, 20 bp, synthetic construct
  misc_feature 1..20: note = Synthetic oligonucleotide
  source 1..20: mol_type = other DNA
ggcaatgaaatgctggcact

SEQ ID NO: 256 - DNA, 20 bp, synthetic construct
  misc_feature 1..20: note = Synthetic oligonucleotide
  source 1..20: mol_type = other DNA
aatgtgagataacgtttgga

SEQ ID NO: 257 - DNA, 20 bp, synthetic construct
  misc_feature 1..20: note = Synthetic oligonucleotide
  source 1..20: mol_type = other DNA
cactgggaaatcaggctgat

SEQ ID NO: 258 - DNA, 20 bp, synthetic construct
  misc_feature 1..20: note = Synthetic oligonucleotide
  source 1..20: mol_type = other DNA
tacaagaacaccttcagaac

SEQ ID NO: 259 - DNA, 20 bp, synthetic construct
  misc_feature 1..20: note = Synthetic oligonucleotide
  source 1..20: mol_type = other DNA
ggagtttgctttgccatcca

SEQ ID NO: 260 - DNA, 20 bp, synthetic construct
  misc_feature 1..20: note = Synthetic oligonucleotide
  source 1..20: mol_type = other DNA
gatggcaaagcaaactcctg

SEQ ID NO: 261 - DNA, 20 bp, synthetic construct
  misc_feature 1..20: note = Synthetic oligonucleotide
  source 1..20: mol_type = other DNA
tgatgggtgctgaagtggca

SEQ ID NO: 262 - DNA, 20 bp, synthetic construct
  misc_feature 1..20: note = Synthetic oligonucleotide
  source 1..20: mol_type = other DNA
tgaaggctttctgttgccac

SEQ ID NO: 263 - DNA, 20 bp, synthetic construct
  misc_feature 1..20: note = Synthetic oligonucleotide
  source 1..20: mol_type = other DNA
tgatggcaaatattagtttc

SEQ ID NO: 264 - DNA, 20 bp, synthetic construct
  misc_feature 1..20: note = Synthetic oligonucleotide
  source 1..20: mol_type = other DNA
caaacgttatctcacattta

SEQ ID NO: 265 - DNA, 20 bp, synthetic construct
  misc_feature 1..20: note = Synthetic oligonucleotide
  source 1..20: mol_type = other DNA
aggctttctgttgccacagg

SEQ ID NO: 266 - DNA, 20 bp, synthetic construct
  misc_feature 1..20: note = Synthetic oligonucleotide
  source 1..20: mol_type = other DNA
taatgactaattgtaggaat

SEQ ID NO: 267 - DNA, 20 bp, synthetic construct
  misc_feature 1..20: note = Synthetic oligonucleotide
  source 1..20: mol_type = other DNA
aggcaatgaaatgctggcac

SEQ ID NO: 268 - DNA, 20 bp, synthetic construct
  misc_feature 1..20: note = Synthetic oligonucleotide
  source 1..20: mol_type = other DNA
agaaccaaatatgaatcatc

SEQ ID NO: 269 - DNA, 20 bp, synthetic construct
  misc_feature 1..20: note = Synthetic oligonucleotide
  source 1..20: mol_type = other DNA
gccttcaggcaatgaaatgc

SEQ ID NO: 270 - DNA, 20 bp, synthetic construct
  misc_feature 1..20: note = Synthetic oligonucleotide
  source 1..20: mol_type = other DNA
aatgctggcactgggaaatc

SEQ ID NO: 271 - DNA, 20 bp, synthetic construct
  misc_feature 1..20: note = Synthetic oligonucleotide
  source 1..20: mol_type = other DNA
tgctttgccatccatgtgac

SEQ ID NO: 272 - DNA, 20 bp, synthetic construct
  misc_feature 1..20: note = Synthetic oligonucleotide
  source 1..20: mol_type = other DNA
gccagcatttcattgcctga

SEQ ID NO: 273 - DNA, 20 bp, synthetic construct
  misc_feature 1..20: note = Synthetic oligonucleotide
  source 1..20: mol_type = other DNA
actgggaaatcaggctgatg

SEQ ID NO: 274 - DNA, 20 bp, synthetic construct
  misc_feature 1..20: note = Synthetic oligonucleotide
  source 1..20: mol_type = other DNA
tgatgttctatgtcaccttc

SEQ ID NO: 275 - DNA, 20 bp, synthetic construct
  misc_feature 1..20: note = Synthetic oligonucleotide
  source 1..20: mol_type = other DNA
tgtggcaacagaaagccttc

SEQ ID NO: 276 - DNA, 20 bp, synthetic construct
  misc_feature 1..20: note = Synthetic oligonucleotide
  source 1..20: mol_type = other DNA
tctaacagatgattcatatt

SEQ ID NO: 277 - DNA, 20 bp, synthetic construct
  misc_feature 1..20: note = Synthetic oligonucleotide
  source 1..20: mol_type = other DNA
ttattcattgtgttatggct

SEQ ID NO: 278 - DNA, 20 bp, synthetic construct
  misc_feature 1..20: note = Synthetic oligonucleotide
  source 1..20: mol_type = other DNA
gcctgtatataatgactaat

SEQ ID NO: 279 - DNA, 20 bp, synthetic construct
  misc_feature 1..20: note = Synthetic oligonucleotide
  source 1..20: mol_type = other DNA
tacaattagtcattatatac

SEQ ID NO: 280 - DNA, 20 bp, synthetic construct
  misc_feature 1..20: note = Synthetic oligonucleotide
  source 1..20: mol_type = other DNA
gcactgggaaatcaggctga

SEQ ID NO: 281 - DNA, 20 bp, synthetic construct
  misc_feature 1..20: note = Synthetic oligonucleotide
  source 1..20: mol_type = other DNA
tgtatataatgactaattgt

SEQ ID NO: 282 - DNA, 20 bp, synthetic construct
  misc_feature 1..20: note = Synthetic oligonucleotide
  source 1..20: mol_type = other DNA
gtgctgaagtggcaaggatg

SEQ ID NO: 283 - DNA, 20 bp, synthetic construct
  misc_feature 1..20: note = Synthetic oligonucleotide
  source 1..20: mol_type = other DNA
gatgattcatatttggttct

SEQ ID NO: 284 - DNA, 20 bp, synthetic construct
  misc_feature 1..20: note = Synthetic oligonucleotide
  source 1..20: mol_type = other DNA
attcagaatcagtgggatga

SEQ ID NO: 285 - DNA, 20 bp, synthetic construct
  misc_feature 1..20: note = Synthetic oligonucleotide
  source 1..20: mol_type = other DNA
tgctgaagtggcaaggatga

SEQ ID NO: 286 - DNA, 20 bp, synthetic construct
  misc_feature 1..20: note = Synthetic oligonucleotide
  source 1..20: mol_type = other DNA
atgattcatatttggttcta

SEQ ID NO: 287 - DNA, 20 bp, synthetic construct
  misc_feature 1..20: note = Synthetic oligonucleotide
  source 1..20: mol_type = other DNA
gcaacataaatgtgagataa

SEQ ID NO: 288 - DNA, 20 bp, synthetic construct
  misc_feature 1..20: note = Synthetic oligonucleotide
  source 1..20: mol_type = other DNA
tggctaggatgatgaacaac

SEQ ID NO: 289 - DNA, 20 bp, synthetic construct
  misc_feature 1..20: note = Synthetic oligonucleotide
  source 1..20: mol_type = other DNA
tggatggcaaagcaaactcc

SEQ ID NO: 290 - DNA, 20 bp, synthetic construct
  misc_feature 1..20: note = Synthetic oligonucleotide
  source 1..20: mol_type = other DNA
gtggcaaggatgaggggata

SEQ ID NO: 291 - DNA, 20 bp, synthetic construct
  misc_feature 1..20: note = Synthetic oligonucleotide
  source 1..20: mol_type = other DNA
ctaacagatgattcatattt

SEQ ID NO: 292 - DNA, 20 bp, synthetic construct
  misc_feature 1..20: note = Synthetic oligonucleotide
  source 1..20: mol_type = other DNA
tgaaagaattcagaatcagt

SEQ ID NO: 293 - DNA, 20 bp, synthetic construct
  misc_feature 1..20: note = Synthetic oligonucleotide
  source 1..20: mol_type = other DNA
ggagctggaggggaggcatc

SEQ ID NO: 294 - DNA, 20 bp, synthetic construct
  misc_feature 1..20: note = Synthetic oligonucleotide
  source 1..20: mol_type = other DNA
ggatatggatattctgctgt

SEQ ID NO: 295 - DNA, 20 bp, synthetic construct
  misc_feature 1..20: note = Synthetic oligonucleotide
  source 1..20: mol_type = other DNA
caggctgatgggtgctgaag

SEQ ID NO: 296 - DNA, 20 bp, synthetic construct
  misc_feature 1..20: note = Synthetic oligonucleotide
  source 1..20: mol_type = other DNA
agttgaaagaattcagaatc

SEQ ID NO: 297 - DNA, 20 bp, synthetic construct
  misc_feature 1..20: note = Synthetic oligonucleotide
  source 1..20: mol_type = other DNA
ttcattgcctgaaggctttc

SEQ ID NO: 298 - DNA, 20 bp, synthetic construct
  misc_feature 1..20: note = Synthetic oligonucleotide
  source 1..20: mol_type = other DNA
gctgaagtggcaaggatgag

SEQ ID NO: 299 - DNA, 20 bp, synthetic construct
  misc_feature 1..20: note = Synthetic oligonucleotide
  source 1..20: mol_type = other DNA
ttgaaagaattcagaatcag

SEQ ID NO: 300 - DNA, 20 bp, synthetic construct
  misc_feature 1..20: note = Synthetic oligonucleotide
  source 1..20: mol_type = other DNA
tacaggctgcctctgggagc

SEQ ID NO: 301 - DNA, 20 bp, synthetic construct
  misc_feature 1..20: note = Synthetic oligonucleotide
  source 1..20: mol_type = other DNA
atatatatgttgatgttcta

SEQ ID NO: 302 - DNA, 20 bp, synthetic construct
  misc_feature 1..20: note = Synthetic oligonucleotide
  source 1..20: mol_type = other DNA
aatatatagtagtaaatgct

SEQ ID NO: 303 - DNA, 20 bp, synthetic construct
  misc_feature 1..20: note = Synthetic oligonucleotide
  source 1..20: mol_type = other DNA
gctgcctctgggagctggag

SEQ ID NO: 304 - DNA, 20 bp, synthetic construct
  misc_feature 1..20: note = Synthetic oligonucleotide
  source 1..20: mol_type = other DNA
aggctgcctctgggagctgg

SEQ ID NO: 305 - DNA, 20 bp, synthetic construct
  misc_feature 1..20: note = Synthetic oligonucleotide
  source 1..20: mol_type = other DNA
cttttttgatggcaaatatt

SEQ ID NO: 306 - DNA, 20 bp, synthetic construct
  misc_feature 1..20: note = Synthetic oligonucleotide
  source 1..20: mol_type = other DNA
gcctcccctccagctcccag

SEQ ID NO: 307 - DNA, 20 bp, synthetic construct
  misc_feature 1..20: note = Synthetic oligonucleotide
  source 1..20: mol_type = other DNA
gcctctgggagctggagggg

SEQ ID NO: 308 - DNA, 20 bp, synthetic construct
  misc_feature 1..20: note = Synthetic oligonucleotide
  source 1..20: mol_type = other DNA
atcaggctgatgggtgctga

SEQ ID NO: 309 - DNA, 20 bp, synthetic construct
  misc_feature 1..20: note = Synthetic oligonucleotide
  source 1..20: mol_type = other DNA
ttttaaatgtaacttccaaa

SEQ ID NO: 310 - DNA, 20 bp, synthetic construct
  misc_feature 1..20: note = Synthetic oligonucleotide
  source 1..20: mol_type = other DNA
ctgttaaattattttcctga

SEQ ID NO: 311 - DNA, 20 bp, synthetic construct
  misc_feature 1..20: note = Synthetic oligonucleotide
  source 1..20: mol_type = other DNA
tccagctcccagaggcagcc

SEQ ID NO: 312 - DNA, 20 bp, synthetic construct
  misc_feature 1..20: note = Synthetic oligonucleotide
  source 1..20: mol_type = other DNA
ggctgcctctgggagctgga

SEQ ID NO: 313 - DNA, 20 bp, synthetic construct
  misc_feature 1..20: note = Synthetic oligonucleotide
  source 1..20: mol_type = other DNA
tctggaggagacattttaaa

SEQ ID NO: 314 - DNA, 20 bp, synthetic construct
  misc_feature 1..20: note = Synthetic oligonucleotide
  source 1..20: mol_type = other DNA
attctttcaactagaataaa

SEQ ID NO: 315 - DNA, 20 bp, synthetic construct
  misc_feature 1..20: note = Synthetic oligonucleotide
  source 1..20: mol_type = other DNA
tgttaaattattttcctgaa

SEQ ID NO: 316 - DNA, 20 bp, synthetic construct
  misc_feature 1..20: note = Synthetic oligonucleotide
  source 1..20: mol_type = other DNA
aaaaaagcaaagaatcctgt

SEQ ID NO: 317 - DNA, 20 bp, synthetic construct
  misc_feature 1..20: note = Synthetic oligonucleotide
  source 1..20: mol_type = other DNA
aaaaattattcattgtgtta

SEQ ID NO: 318 - DNA, 20 bp, synthetic construct
  misc_feature 1..20: note = Synthetic oligonucleotide
  source 1..20: mol_type = other DNA
ttttaaataagcaacataaa

SEQ ID NO: 319 - DNA, 20 bp, synthetic construct
  misc_feature 1..20: note = Synthetic oligonucleotide
  source 1..20: mol_type = other DNA
aaattttatatatatgttga

SEQ ID NO: 320 - DNA, 20 bp, synthetic construct
  misc_feature 1..20: note = Synthetic oligonucleotide
  source 1..20: mol_type = other DNA
aggaaaaataaatatatagt

SEQ ID NO: 321 - DNA, 20 bp, synthetic construct
  misc_feature 1..20: note = Synthetic oligonucleotide
  source 1..20: mol_type = other DNA
atttaaaaaattattcattg

SEQ ID NO: 322 - DNA, 20 bp, synthetic construct
  misc_feature 1..20: note = Synthetic oligonucleotide
  source 1..20: mol_type = other DNA
gagttaaaattttatatata

SEQ ID NO: 323 - DNA, 20 bp, synthetic construct
  misc_feature 1..20: note = Synthetic oligonucleotide
  source 1..20: mol_type = other DNA
tttggaagttacatttaaaa

SEQ ID NO: 324 - DNA, 20 bp, synthetic construct
  misc_feature 1..20: note = Synthetic oligonucleotide
  source 1..20: mol_type = other DNA
atcaaaaaagcaaagaatcc

SEQ ID NO: 325 - DNA, 20 bp, synthetic construct
  misc_feature 1..20: note = Synthetic oligonucleotide
  source 1..20: mol_type = other DNA
aggattctttgcttttttga

SEQ ID NO: 326 - DNA, 20 bp, synthetic construct
  misc_feature 1..20: note = Synthetic oligonucleotide
  source 1..20: mol_type = other DNA
ttatttaaaaaattattcat

SEQ ID NO: 327 - DNA, 20 bp, synthetic construct
  misc_feature 1..20: note = Synthetic oligonucleotide
  source 1..20: mol_type = other DNA
aaaaggaaaaataaatatat

SEQ ID NO: 328 - DNA, 20 bp, synthetic construct
  misc_feature 1..20: note = Synthetic oligonucleotide
  source 1..20: mol_type = other DNA
tttatttttccttttattct

SEQ ID NO: 329 - DNA, 20 bp, synthetic construct
  misc_feature 1..20: note = Synthetic oligonucleotide
  source 1..20: mol_type = other DNA
tggaaggagggtccctatac

SEQ ID NO: 330 - DNA, 20 bp, synthetic construct
  misc_feature 1..20: note = Synthetic oligonucleotide
  source 1..20: mol_type = other DNA
ggtatctttgatactaacct

SEQ ID NO: 331 - DNA, 20 bp, synthetic construct
  misc_feature 1..20: note = Synthetic oligonucleotide
  source 1..20: mol_type = other DNA
actgttgcctccggttctga

SEQ ID NO: 332 - DNA, 20 bp, synthetic construct
  misc_feature 1..20: note = Synthetic oligonucleotide
  source 1..20: mol_type = other DNA
ccttcagaaccggaggcaac

SEQ ID NO: 333 - DNA, 20 bp, synthetic construct
  misc_feature 1..20: note = Synthetic oligonucleotide
  source 1..20: mol_type = other DNA
agctgagcaggtcttaggac

SEQ ID NO: 334 - DNA, 20 bp, synthetic construct
  misc_feature 1..20: note = Synthetic oligonucleotide
  source 1..20: mol_type = other DNA
ctgttgcctccggttctgaa

SEQ ID NO: 335 - DNA, 20 bp, synthetic construct
  misc_feature 1..20: note = Synthetic oligonucleotide
  source 1..20: mol_type = other DNA
ccagagccaagcttgagtca

SEQ ID NO: 336 - DNA, 20 bp, synthetic construct
  misc_feature 1..20: note = Synthetic oligonucleotide
  source 1..20: mol_type = other DNA
tcagctttaacgtgattttc

SEQ ID NO: 337 - DNA, 20 bp, synthetic construct
  misc_feature 1..20: note = Synthetic oligonucleotide
  source 1..20: mol_type = other DNA
ctccttccatgactcaagct

SEQ ID NO: 338 - DNA, 20 bp, synthetic construct
  misc_feature 1..20: note = Synthetic oligonucleotide
  source 1..20: mol_type = other DNA
ttggattgcatctactgtat

SEQ ID NO: 339 - DNA, 20 bp, synthetic construct
  misc_feature 1..20: note = Synthetic oligonucleotide
  source 1..20: mol_type = other DNA
gctcactgagccttgaactc

SEQ ID NO: 340 - DNA, 20 bp, synthetic construct
  misc_feature 1..20: note = Synthetic oligonucleotide
  source 1..20: mol_type = other DNA
actcaagcttggctctggcc

SEQ ID NO: 341 - DNA, 20 bp, synthetic construct
  misc_feature 1..20: note = Synthetic oligonucleotide
  source 1..20: mol_type = other DNA
tggattgcatctactgtata

SEQ ID NO: 342 - DNA, 20 bp, synthetic construct
  misc_feature 1..20: note = Synthetic oligonucleotide
  source 1..20: mol_type = other DNA
acaggccagagccaagcttg

SEQ ID NO: 343 - DNA, 20 bp, synthetic construct
  misc_feature 1..20: note = Synthetic oligonucleotide
  source 1..20: mol_type = other DNA
ttcttttggattgcatctac

SEQ ID NO: 344 - DNA, 20 bp, synthetic construct
  misc_feature 1..20: note = Synthetic oligonucleotide
  source 1..20: mol_type = other DNA
ttcattcaactgttgcctcc

SEQ ID NO: 345 - DNA, 20 bp, synthetic construct
  misc_feature 1..20: note = Synthetic oligonucleotide
  source 1..20: mol_type = other DNA
ccatgactcaagcttggctc

SEQ ID NO: 346 - DNA, 20 bp, synthetic construct
  misc_feature 1..20: note = Synthetic oligonucleotide
  source 1..20: mol_type = other DNA
gtatctttgatactaacctt

SEQ ID NO: 347 - DNA, 20 bp, synthetic construct
  misc_feature 1..20: note = Synthetic oligonucleotide
  source 1..20: mol_type = other DNA
ctgaaatgaacagtagactt

SEQ ID NO: 348 - DNA, 20 bp, synthetic construct
  misc_feature 1..20: note = Synthetic oligonucleotide
  source 1..20: mol_type = other DNA
caagcttgagtcatggaagg

SEQ ID NO: 349 - DNA, 20 bp, synthetic construct
  misc_feature 1..20: note = Synthetic oligonucleotide
  source 1..20: mol_type = other DNA
tttcattcaactgttgcctc

SEQ ID NO: 350 - DNA, 20 bp, synthetic construct
  misc_feature 1..20: note = Synthetic oligonucleotide
  source 1..20: mol_type = other DNA
tccttagcttccagccattg

SEQ ID NO: 351 - DNA, 20 bp, synthetic construct
  misc_feature 1..20: note = Synthetic oligonucleotide
  source 1..20: mol_type = other DNA
cttccttagcttccagccat

SEQ ID NO: 352 - DNA, 20 bp, synthetic construct
  misc_feature 1..20: note = Synthetic oligonucleotide
  source 1..20: mol_type = other DNA
aaaggattcaacacaatggc

SEQ ID NO: 353 - DNA, 20 bp, synthetic construct
  misc_feature 1..20: note = Synthetic oligonucleotide
  source 1..20: mol_type = other DNA
aacacaatggctggaagcta

SEQ ID NO: 354 - DNA, 20 bp, synthetic construct
  misc_feature 1..20: note = Synthetic oligonucleotide
  source 1..20: mol_type = other DNA
tcactgagccttgaactctg

SEQ ID NO: 355 - DNA, 20 bp, synthetic construct
  misc_feature 1..20: note = Synthetic oligonucleotide
  source 1..20: mol_type = other DNA
tgttaaaggattcaacacaa

SEQ ID NO: 356 - DNA, 20 bp, synthetic construct
  misc_feature 1..20: note = Synthetic oligonucleotide
  source 1..20: mol_type = other DNA
catcattaaattacaatcta

SEQ ID NO: 357 - DNA, 20 bp, synthetic construct
  misc_feature 1..20: note = Synthetic oligonucleotide
  source 1..20: mol_type = other DNA
gaagaagctgagcaggtctt

SEQ ID NO: 358 - DNA, 20 bp, synthetic construct
  misc_feature 1..20: note = Synthetic oligonucleotide
  source 1..20: mol_type = other DNA
aaatcacagaaaccaaggtt

SEQ ID NO: 359 - DNA, 20 bp, synthetic construct
  misc_feature 1..20: note = Synthetic oligonucleotide
  source 1..20: mol_type = other DNA
agcttgagtcatggaaggag

SEQ ID NO: 360 - DNA, 20 bp, synthetic construct
  misc_feature 1..20: note = Synthetic oligonucleotide
  source 1..20: mol_type = other DNA
cctttaacatttcattcaac

SEQ ID NO: 361 - DNA, 20 bp, synthetic construct
  misc_feature 1..20: note = Synthetic oligonucleotide
  source 1..20: mol_type = other DNA
actgttcatttcagctttaa

SEQ ID NO: 362 - DNA, 20 bp, synthetic construct
  misc_feature 1..20: note = Synthetic oligonucleotide
  source 1..20: mol_type = other DNA
agccaagcttgagtcatgga

SEQ ID NO: 363 - DNA, 20 bp, synthetic construct
  misc_feature 1..20: note = Synthetic oligonucleotide
  source 1..20: mol_type = other DNA
acgttaaagctgaaatgaac

SEQ ID NO: 364 - DNA, 20 bp, synthetic construct
  misc_feature 1..20: note = Synthetic oligonucleotide
  source 1..20: mol_type = other DNA
gaggcaacagttgaatgaaa

SEQ ID NO: 365 - DNA, 20 bp, synthetic construct
  misc_feature 1..20: note = Synthetic oligonucleotide
  source 1..20: mol_type = other DNA
gataaaattataccatagat

SEQ ID NO: 366 - DNA, 20 bp, synthetic construct
  misc_feature 1..20: note = Synthetic oligonucleotide
  source 1..20: mol_type = other DNA
aagcttgagtcatggaagga

SEQ ID NO: 367 - DNA, 20 bp, synthetic construct
  misc_feature 1..20: note = Synthetic oligonucleotide
  source 1..20: mol_type = other DNA
atcattaaattacaatctat

SEQ ID NO: 368 - DNA, 20 bp, synthetic construct
  misc_feature 1..20: note = Synthetic oligonucleotide
  source 1..20: mol_type = other DNA
catagattgtaatttaatga

SEQ ID NO: 369 - DNA, 20 bp, synthetic construct
  misc_feature 1..20: note = Synthetic oligonucleotide
  source 1..20: mol_type = other DNA
taattttatcaaatgtaacc

SEQ ID NO: 370 - DNA, 20 bp, synthetic construct
  misc_feature 1..20: note = Synthetic oligonucleotide
  source 1..20: mol_type = other DNA
aataaatatacaaagtctac

SEQ ID NO: 371 - DNA, 20 bp, synthetic construct
  misc_feature 1..20: note = Synthetic oligonucleotide
  source 1..20: mol_type = other DNA
gctaaggaagaagctgagca

SEQ ID NO: 372 - DNA, 20 bp, synthetic construct
  misc_feature 1..20: note = Synthetic oligonucleotide
  source 1..20: mol_type = other DNA
agctaaggaagaagctgagc

SEQ ID NO: 373 - DNA, 20 bp, synthetic construct
  misc_feature 1..20: note = Synthetic oligonucleotide
  source 1..20: mol_type = other DNA
ctcactgagccttgaactct

SEQ ID NO: 374 - DNA, 20 bp, synthetic construct
  misc_feature 1..20: note = Synthetic oligonucleotide
  source 1..20: mol_type = other DNA
acagttgaatgaaatgttaa

SEQ ID NO: 375 - DNA, 20 bp, synthetic construct
  misc_feature 1..20: note = Synthetic oligonucleotide
  source 1..20: mol_type = other DNA
ttgatactaaccttggtttc

SEQ ID NO: 376 - DNA, 20 bp, synthetic construct
  misc_feature 1..20: note = Synthetic oligonucleotide
  source 1..20: mol_type = other DNA
aagttattaacagaaaatca

SEQ ID NO: 377 - DNA, 20 bp, synthetic construct
  misc_feature 1..20: note = Synthetic oligonucleotide
  source 1..20: mol_type = other DNA
agccccagagttcaaggctc

SEQ ID NO: 378 - DNA, 20 bp, synthetic construct
  misc_feature 1..20: note = Synthetic oligonucleotide
  source 1..20: mol_type = other DNA
tatggtataattttatcaaa

SEQ ID NO: 379 - DNA, 20 bp, synthetic construct
  misc_feature 1..20: note = Synthetic oligonucleotide
  source 1..20: mol_type = other DNA
tgtaatttaatgatgtttaa

SEQ ID NO: 380 - DNA, 20 bp, synthetic construct
  misc_feature 1..20: note = Synthetic oligonucleotide
  source 1..20: mol_type = other DNA
tttaatgatgtttaatgtaa

SEQ ID NO: 381 - DNA, 20 bp, synthetic construct
  misc_feature 1..20: note = Synthetic oligonucleotide
  source 1..20: mol_type = other DNA
tggtttctgtgattttcttt

SEQ ID NO: 382 - DNA, 20 bp, synthetic construct
  misc_feature 1..20: note = Synthetic oligonucleotide
  source 1..20: mol_type = other DNA
aaagaaaatcacagaaacca

SEQ ID NO: 383 - DNA, 20 bp, synthetic construct
  misc_feature 1..20: note = Synthetic oligonucleotide
  source 1..20: mol_type = other DNA
aagaaaatcacagaaaccaa

SEQ ID NO: 384 - DNA, 20 bp, synthetic construct
  misc_feature 1..20: note = Synthetic oligonucleotide
  source 1..20: mol_type = other DNA
acctttttaaaataaaatac

SEQ ID NO: 385 - DNA, 20 bp, synthetic construct
  misc_feature 1..20: note = Synthetic oligonucleotide
  source 1..20: mol_type = other DNA
cctttttaaaataaaatact

SEQ ID NO: 386 - DNA, 20 bp, synthetic construct
  misc_feature 1..20: note = Synthetic oligonucleotide
  source 1..20: mol_type = other DNA
ccagtattttattttaaaaa

SEQ ID NO: 387 - DNA, 20 bp, synthetic construct
  misc_feature 1..20: note = Synthetic oligonucleotide
  source 1..20: mol_type = other DNA
accagtattttattttaaaa

SEQ ID NO: 388 - DNA, 20 bp, synthetic construct
  misc_feature 1..20: note = Synthetic oligonucleotide
  source 1..20: mol_type = other DNA
aaggctcagtgagctatgat

SEQ ID NO: 389 - DNA, 20 bp, synthetic construct
  misc_feature 1..20: note = Synthetic oligonucleotide
  source 1..20: mol_type = other DNA
ctaagaaaataaatatacaa

(19) United States
(12) Patent Application Publication
(10) Pub. No.: US 2025/0179450 A1
(43) Pub. Date: Jun. 5, 2025

(54) CRISPR/SpCas9 VARIANT AND METHODS FOR ENHANCED CORRECTION OF DUCHENNE MUSCULAR DYSTROPHY MUTATIONS

(71) Applicant: THE BOARD OF REGENTS OF THE UNIVERSITY OF TEXAS SYSTEM, Austin, TX (US)

(72) Inventors: Yu ZHANG, Dallas, TX (US); Eric OLSON, Dallas, TX (US); Rhonda BASSEL-DUBY, Dallas, TX (US)

(73) Assignee: THE BOARD OF REGENTS OF THE UNIVERSITY OF TEXAS SYSTEM, Austin, TX (US)

(21) Appl. No.: 18/839,119

(22) PCT Filed: Feb. 16, 2023
(86) PCT No.: PCT/US23/62712

§ 371 Date: Aug. 16, 2024

Related U.S. Application Data
(60) Provisional application No. 63/311,275, filed on Feb. 17, 2022.

(51) Int. Cl. C12N 9/22; A61K 38/46; A61K 48/00; A61P 21/00; C12N 15/11; C12N 15/86

(52) U.S. Cl. CPC C12N 9/22; A61K 38/465; A61K 48/0058; A61K 48/0075; A61P 21/00; C12N 15/11; C12N 15/86; C12N 2310/20; C12N 2750/14143; C12N 2830/008

(57) ABSTRACT

CRISPR/Cas9-mediated genome editing holds clinical potential for treating genetic diseases, such as Duchenne muscular dystrophy (DMD), which is caused by mutations in the dystrophin gene. Here, an improved variant of SpCas9 is provided that exhibits increase capacity to target genomic loci and drive CRISPR-mediated gene editing and a higher efficiency, particular for the treatment of DMD.

### PRIORITY CLAIM

This application claims benefit of priority to U.S. Provisional Application Ser. No. 63/311,275, filed Feb. 17, 2022, the entire contents of which are hereby incorporated by reference.

### FEDERAL FUNDING SUPPORT CLAUSE

This invention was made with government support under grant no. P50HD087351 awarded by National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Feb. 3, 2023, is named UTFD3836WO.xml and is 496 kilobytes in size.

### FIELD OF THE DISCLOSURE

The present disclosure relates to the fields of molecular biology, medicine and genetics. More particularly, the disclosure relates to compositions and uses thereof for genome editing to correct mutations in vivo using an exon-skipping and/or reframing approach.

### BACKGROUND

Muscular dystrophies (MD) are a group of more than 30 genetic diseases characterized by progressive weakness and degeneration of the skeletal muscles that control movement. Duchenne muscular dystrophy (DMD) is one of the most severe forms of MD that affects approximately 1 in 5000 boys and is characterized by progressive muscle weakness and premature death. Cardiomyopathy and heart failure are common, incurable and lethal features of DMD. The disease is caused by mutations in the gene encoding dystrophin (DMD), a large intracellular protein that links the dystroglycan complex at the cell surface with the underlying cytoskeleton, thereby maintaining integrity of the muscle cell membrane during contraction. Mutations in the dystrophin gene result in loss of expression of dystrophin, causing muscle membrane fragility and progressive muscle wasting.

### SUMMARY

Despite intense efforts to find cures through a variety of approaches, including myoblast transfer, viral delivery, and oligonucleotide-mediated exon skipping, there remains no cure for any type of muscular dystrophy. The disclosure provides for the use of an improved SpCas9 variant to increase the efficiency of CRISPR-mediated interventions in treatment of muscular dystrophy.

In some embodiments, there is provided a composition comprising (a) a first vector comprising a nucleic acid comprising
a sequence encoding a first DMD guide RNA targeting a first genomic target sequence; a sequence encoding a first promoter, wherein the first promoter drives expression of the sequence encoding the first DMD guide RNA, and
(b) a second vector comprising a nucleic acid comprising:
   a sequence encoding an SpCas9 or a nuclease domain thereof, wherein said SpCas9 or nuclease domain thereof comprises Leucine or Valine at residue 1135, Arginine, Lysine or Serine at residue 1218, Valine, Glutamine or Phenylalanine at residue 1219, Glutamine or Valine at residue 1335 and Arginine at residue 1337 corresponding to the sequence shown in FIG. 3;
(c) a sequence encoding a muscle-specific promoter, wherein the muscle-specific promoter drives expression of the sequence encoding said SpCas9 or a nuclease domain thereof.

In embodiments, the ratio of the first vector to the second vector is 1:1 to 1:30 or from 1:1 to 30:1; or the ratio of the first vector to the second vector is 1:2, 1:3, 1:4, 1:5, 1:6, 1:7, 1:8, 1:9, 1:10, 1:15, 1:20 or 1:25; or the ratio of the second vector to the first vector is 1:2, 1:3, 1:4, 1:5, 1:6, 1:7, 1:8, 1:9, 1:10, 1:15, 1:20 or 1:25. The first vector and/or the second vector may comprise a sequence isolated or derived from an adeno-associated virus (AAV). The sequence encoding the muscle-specific promoter may comprise or consist of a sequence encoding a CK8 promoter, such as a CK8e promoter. The DMD guide RNA may comprise or consist of the sequence 5′-CUGUUGCCUCCGGUUCUGAA-3′ (SEQ ID NO: 1). The Ca9 nuclease or nuclease domain is derived from Staphylococcus pyogenes.

The composition may comprises between 5×10¹¹ viral genomes (vg)/kilogram (kg) and 1×10¹⁵ vg/kg, inclusive of the endpoints, of the first vector, such as at least 5×10¹² viral genomes (vg)/kilogram (kg), at least 1×10¹³ viral genomes (vg)/kilogram (kg), at least 5×10¹³ viral genomes (vg)/kilogram (kg), at least 1×10¹⁴ viral genomes (vg)/kilogram (kg), at least 5×10¹⁴ viral genomes (vg)/kilogram (kg) of the first vector, or at least 1×10¹⁵ viral genomes (vg)/kilogram (kg) of the first vector.

The Cas9 nuclease or nuclease domain thereof may have the mutations D1135L, G1218R, E1219V, R1335Q, and T1337R as compared to the wild-type sequence shown in FIG. 3 and SEQ ID NO: 2. In embodiments, the Cas9 nuclease comprises the sequence of SEQ ID NO: 6.

The composition may further comprise a pharmaceutically acceptable carrier.

Also provided is a method of treating muscular dystrophy in a subject in need thereof comprising administering to the subject a therapeutically effective amount of the composition described herein. The composition may be administered locally. The composition may be administered directly to a muscle tissue. The composition may be administered by an intramuscular infusion or injection. The muscle tissue may comprise a tibialis anterior tissue, a quadriceps tissue, a soleus tissue, a diaphragm tissue, or a heart tissue. The composition may be administered by intra-cardiac injection. The composition may be administered systemically, such as by an intravenous infusion or injection.

The subject may be a neonate, an infant, a child, a young adult, or an adult. The subject may have muscular dystrophy. The subject maybe a genetic carrier for muscular dystrophy. The subject may be male, female, an adult, such as an adult at least 18 years old or at least 25 years old. The adult may be at least 20 kg. The subject may be a child, such as less than 18 years of age. The child may be 20 kg or less. The subject is an infant, such as less than 2 years old.

Upon administering the therapeutically effective amount of the composition, the subject produces a minimal immune response to the composition. The minimal immune response to the composition may be reduced or eliminated treatment with an anti-inflammatory agent or an immune suppressive agent. The composition may not induce breaks in a predicted alternative targeting site. The predicted alternative targeting site comprises a coding sequence of the human genome and wherein the coding sequence may comprise at least 2, 3, 4, 5, 6, 7, 8, 9, or 10 mismatches with respect to the first genomic target sequence.

The administration of the therapeutically effective amount of the composition may be provided as a single dose or provided within a single medical procedure. The administration of the therapeutically effective amount of the composition may be provided as multiple doses or provided over multiple medical procedures.

In yet another embodiment, there is provided a Cas9 nuclease or nuclease domain comprising Leucine or Valine at residue 1135, Arginine, Lysine or Serine at residue 1218, Valine, Glutamine or Phenylalanine at residue 1219, Glutamine or Valine at residue 1335 and/or Arginine at residue 1337 corresponding to the sequence shown in FIG. 3. The Cas9 nuclease or nuclease domain may comprise Leucine or Valine at residue 1135 corresponding to the sequence of SEQ ID NO: 2. The Cas9 nuclease or nuclease domain may comprise Arginine, Lysine or Serine at residue 1218 corresponding to the sequence of SEQ ID NO: 2. The Cas9 nuclease or nuclease domain may comprise Valine, Glutamine or Phenylalanine at residue 1219 corresponding to the sequence of SEQ ID NO: 2. The Cas9 nuclease or nuclease domain may comprise Glutamine or Valine at residue 1335 corresponding to the sequence of SEQ ID NO: 2. The Cas9 nuclease or nuclease domain may comprise Arginine at residue 1337 corresponding to the sequence of SEQ ID NO: 2. The Cas9 nuclease or nuclease domain may comprise a sequence that is at least 85%, 90%, 91%, 92%, 93%, 94%, 95%, 96%, 97%, 98%, 99%, or 100% identical the sequence of SEQ ID NO: 6, or biologically active fragments thereof.

The Cas9 nuclease may be the mutations D1135L, G1218R, E1219V, R1335Q, and/or T1337R as compared to the wild-type sequence in FIG. 3 and SEQ ID NO: 2. In embodiments, the Cas9 nuclease comprises the sequence of SEQ ID NO: 6. The Cas9 nuclease or nuclease domain is derived from Staphylococcus pyogenes.

Further provided is a nucleic acid encoding a Cas9 nuclease or nuclease domain comprising Leucine or Valine at residue 1135, Arginine, Lysine or Serine at residue 1218, Valine, Glutamine or Phenylalanine at residue 1219, Glutamine or Valine at residue 1335 and/or Arginine at residue 1337 corresponding to the sequence shown in FIG. 3, or SEQ ID NO: 2. The nucleic acid may encode a Cas9 nuclease wherein the Cas9 nuclease has the mutations D1135L, G1218R, E1219V, R1335Q, and T1337R, a Cas9 nuclease comprising the sequence of SEQ ID NO: 6, and/or a Cas9 nuclease or nuclease domain derived from Staphylococcus pyogenes. The nucleic may be an RNA or a DNA. The nucleic acid may be located in a vector under the control of a promoter active in eukaryotic cells, such as an AAV vector. The promoter may be a muscle-specific promoter, such as a CK8/CK8e promoter.

Also provided is an sgRNA comprising the sequence 5′-CUGUUGCCUCCGGUUCUGAA-3′(SEQ ID NO: 1). Further provided is a nuclecic acid comprising the sgRNA comprising the sequence 5′-CUGUUGCCUCCGGUUCUGAA-3′ (SEQ ID NO: 1). The sgRNA or nucleic acid may belocated in a vector under the control of a promoter active in eukaryotic cells, such as an AAV vector.

Further provided is a host cell comprising a nucleic acid described herein.

In some embodiments of the method of treating of the disclosure, the administration of the therapeutically effective amount of the composition is provided as a single dose or provided within a single medical procedure.

In some embodiments of the method of treating of the disclosure, the administration of the therapeutically effective amount of the composition is provided as multiple doses or provided over multiple medical procedures.

As used herein in the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, for the method being employed to determine the value, or that exists among the study subjects. Such an inherent variation may be a variation of ±10% of the stated value.

As used herein, “reframing” is used to refer to a genome editing strategy in which small INDELs restore the protein reading frame. The term “skipping” or “exon skipping” is used to refer to a genome editing strategy wherein a splice donor or splice acceptor site is disrupted, which results in exon skipping and restoration of the protein reading frame.

Throughout this application, nucleotide sequences are listed in the 5′ to 3′ direction, and amino acid sequences are listed in the N-terminal to C-terminal direction, unless indicated otherwise.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. Strategies for CRISPR/LRVQR-SpCas9-mediated genome editing of DMD Ex53. (FIG. 1A) An out-of-frame deletion of DMD exon 52 results in splicing of exon 51 to 53, generating a premature stop codon in exon 53. A CRISPR/LRVQR-SpCas9-mediated “single-cut” strategy was designed to restore the open reading frame (ORF) of the DMD gene. If the genomic insertions and deletions (INDELs) result in one nucleotide insertion (3n+1), exon 53 will be reframed with adjacent exon 51 and 54. If the INDEL is large enough to delete the 5′-AG-3′ splice acceptor sequence, exon 53 will be skipped, resulting in splicing of exon 51 to exon 54. (FIG. 1B) Illustration of CRISPR/LRVQR-SpCas9 sgRNA targeting DMD exon 53. This sgRNA recognizes a 5′-GGTG-3′ PAM in exon 53 and generates a cut 18 base pairs upstream of the premature TGA stop codon (SEQ ID NOS: 3 and 4).

FIGS. 2A-E. Genome editing of DMD exon 53 by WT-SpCas9 and LRVQR-SpCas9. (FIG. 2A) TIDE (Tracking of Indels by Decomposition) analysis of WT-SpCas9-mediated editing of DMD exon 53 shows high level of nonproductive editing events in DMD exon 53. (FIG. 2B) Decomposition graph of WT-SpCas9-mediated editing of DMD exon 53 shows high quality of sequencing readout. (FIG. 2C) TIDE analysis of LRVQR-SpCas9-mediated editing of DMD exon 53 shows high level of one nucleotide insertion event in DMD exon 53. (FIG. 2D) Decomposition graph of LRVQR-SpCas9-mediated editing of DMD exon 53 shows high quality of sequencing readout. (FIG. 2E) Quantification of genome editing events by WT-SpCas9 and LRVQR-SpCas9 shows LRVQR-SpCas9-mediated editing of DMD exon 53 significantly increases productive editing events while reducing nonproductive editing events.

FIG. 3. WT SpCas9 sequence (SEQ ID NO: 2).

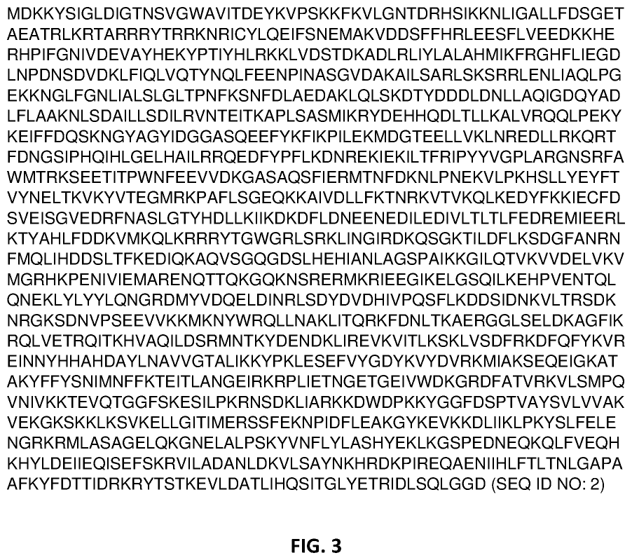

FIG. 4. LRVQR SpCas9 sequence (SEQ ID NO: 6). LRVQR SpCas9 sequence mutations are highlighted in bold/underline.

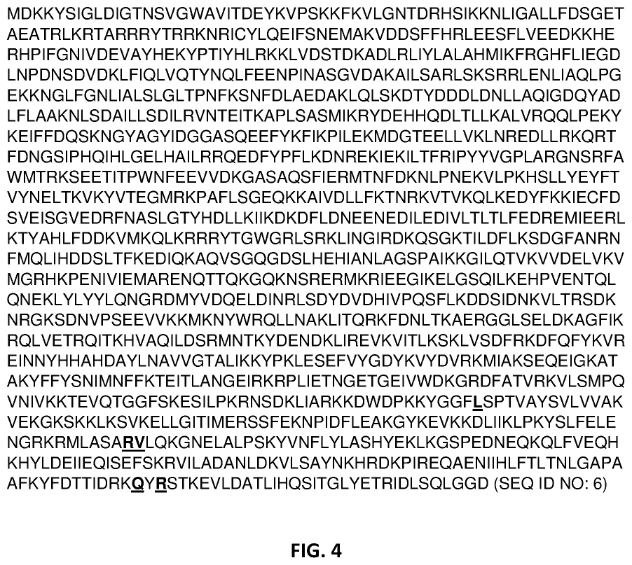

FIGS. 5A-C. LRVQR-SpCas9-mediated single cut gene editing restores dystrophin expression in patient iPSC-derived DMD ΔEx52 cardiomyocytes. (FIG. 5A) DMD ΔEx52 iPSCs were edited by LRVQR-SpCas9 gene editing components (corrected DMD iPSCs) and then differentiated into corrected cardiomyocytes (CMs) for downstream analysis. (FIG. 5B) Western blot shows dystrophin protein restoration in DMD ΔEx52 CMs following LRVQR-SpCas9-mediated single cut gene editing. Vinculin was used as the loading control. (C) Immunocytochemistry shows dystrophin restoration in DMD ΔEx52 CMs following LRVQR-SpCas9-mediated single cut gene editing. Red, dystrophin immunostaining; green, troponin I immunostaining. Scale bar, 100 μm.

FIGS. 6A-B. Design of AAV-based vectors to deliver CRISPR/LRVQR-SpCas9 gene editing components. Dual AAV delivery systems to package the LRVQR-SpCas9 gene editing components individually allow the efficiency of the LRVQR spCas9 nuclease and sgRNA to be assessed in vivo. (FIG. 6A). A double-stranded self-complementary AAV (scAAV) vector expresses the sgRNA, including two copies of the sgRNA expression cassette driven by two RNA polymerase III promoters, U6 and M11 (FIG. 6B).

FIG. 7. Systemic CRISPR/LRVQR-SpCas9 gene editing restores dystrophin expression in humanized DMD mice. Immunohistochemistry results of AAV-SpCas9-LRVQR and AAV-sgRNA given to postnatal day 4 (P4) Δ52;h53KI mice through intraperitoneal (IP) injection shows extensive dystrophin expression in skeletal muscles and the heart.

FIG. 8. Western blot analysis of humanized DMD mice receiving systemic CRISPR/LRVQR-SpCas9 gene editing shows dystrophin restoration. Western blot analysis shows restored dystrophin protein expression in multiple skeletal muscles and heart in mice receiving the systemic AAV-CRISPR/LRVQR-SpCas9 treatment.

FIG. 9A-B. Systemic CRISPR/LRVQR-SpCas9 gene editing improves muscle function in humanized DMD mice. Analysis of serum CK levels and forelimb grip strength demonstrates the effects of dystrophin restoration on muscle integrity and function in CRISPR/LRVQR-SpCas9 gene edited Δ52;h53KI DMD mice (FIG. 9A). Quantification of grip strength of Δ52;h53KI DMD mice receiving saline shows a reduced by 65% compared to h53KI control mice, whereas Δ52;h53KI DMD mice receiving systemic AAV-LRVQR-SpCas9 gene editing show a significant improvement of forelimb grip strength (FIG. 9B).

### DETAILED DESCRIPTION

DMD is a new mutation syndrome with more than 4,000 independent mutations that have been identified in humans. The majority of patient mutations include deletions that cluster in a hotspot, and thus a therapeutic approach for skipping and/or reframing certain exon applies to large group of patients. The rationale of the exon skipping and/or reframing approach is based on the genetic difference between DMD and Becker muscular dystrophy (BMD) patients. In DMD patients, the reading frame of dystrophin mRNA is disrupted resulting in prematurely truncated, non-functional dystrophin proteins. BMD patients have mutations in the DMD gene that maintain the reading frame allowing the production of internally deleted, but partially functional dystrophins leading to much milder disease symptoms compared to DMD patients.

The disclosure provides improved Clustered Regularly Interspaced Short Palindromic Repeat/Cas9 (CRISPR/Cas9)-mediated genome editing compositions for correcting a dystrophin gene (DMD) mutation or for use in a method of correcting a dystrophin gene (DMD) mutation, a mutation which left untreated, results in the onset of DMD. The data presented herein show that using a variant of SpCas9 the efficiency of removal of mutant sequences by reframing and/or exon skipping in muscle cells of mice, dogs and humans is increased while maintaining safe and effective therapy with minimal off-targeting and immunogenetic effects.

These and other aspects of the disclosure are reproduced below.

### I. CRISPR SYSTEMS

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

A. Guide RNA (gRNA)

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type III promoter U6. Synthetic gRNAs are slightly over 100 bp at the minimum length and contain a portion which is targets the 20 protospacer nucleotides immediately preceding the PAM sequence NGG; gRNAs do not contain a PAM sequence.

In some embodiments, the gRNA targets a site within a wild-type dystrophin gene. An exemplary wild-type dystrophin sequence includes the human sequence (see GenBank Accession NO. NC_000023.11), located on the human X chromosome, which codes for the protein dystrophin (GenBank Accession No. AAA53189; SEQ ID NO: 7), the sequence of which is reproduced below:

```
      1 MLWWEEVEDC YEREDVQKKT FTKWVNAQFS KFGKQHIENL FSDLQDGRRL LDLLEGLIGQ
    61 KLPKEKGSTR VHALNNVNKA LRVLQNNNVD LVNIGSTDIV DGNHKLTLGL IWNIILHWQV
  121 KNVMKNIMAG LQQTNSEKIL LSWVRQSTRN YPQVNVINFT TSWSDGLALN ALIHSHRPDL
  181 FDWNSVVCQQ SATQRLEHAF NIARYQLGIE KLLDPEDVDT TYPDKKSILM YITSLFQVLP
  241 QQVSIEAIQE VEMLPRPPKV TKEEHFQLHH QMHYSQQITV SLAQGYERTS SPKPRFKSYA
  301 YTQAAYVITS DPTRSPFPSQ HLEAPEDKSF GSSLMESEVN LDRYQTALEE VLSWLLSAED
  361 TLQAQGEISN DVEVVKDQFH THEGYMMDLT AHQGRVGNIL QLGSKLIGTG KLSEDEETEV
  421 QEQMNLLNSR WECLRVASME KQSNLHRVLM DLQNQKLKEL NDWLIKTEER TRKMEEEPLG
  481 PDLEDLKRQV QQHKVLQEDL EQEQVRVNSL THMVVVVDES SGDHATAALE EQLKVLGDRW
  541 ANICRWTEDR WVLLQDILLK WQRLTEEQCL FSAWLSEKED AVNKIHTTGF KDQNEMLSSL
  601 QKLAVLKADL EKKKQSMGKL YSLKQDLLST LKNKSVTQKT EAWLDNFARC WDNLVQKLEK
  661 STAQISQAVT TTQPSLTQTT VMETVTTVIT REQILVKHAQ EELPPPPPQK KRQITVDSEI
  721 RKRLDVDITE LHSWITRSEA VLQSPEFAIF RKEGNFSDLK EKVNAIEREK AEKFRKLQDA
  781 SRSAQALVEQ MVNEGVNADS IKQASEQLNS RWIEFCQLLS ERLNWLEYQN NIIAFYNQLQ
  841 QLEQMTTTAE NWLKIQPTTP SEPTAIKSQL KICKDEVNRL SGLQPQIERL KIQSIALKEK
  901 GQGPMFLDAD FVAFTNHFKQ VFSDVQAREK ELQTIFDTLP PMRYQETMSA IRTWVQQSET
  961 KLSIPQLSVT DYEIMEQRLG ELQALQSSLQ EQQSGLYYLS TTVKEMSKKA PSEISRKYQS
1021 EFEEIEGRWK KLSSQLVEHC QKLEEQMNKL RKIQNHIQTL KKWMAEVDVF LKEEWPALGD
1081 SEILKKQLKQ CRLLVSDIQT IQPSLNSVNE GGQKIKNEAE PEFASRLETE LKELNTQWDH
1141 MCQQVYARKE ALKGGLEKTV SLQKDLSEMH EWMTQAEEEY LERDFEYKTP DELQKAVEEM
1201 KRAKEEAQQK EAKVKLLTES VNSVIAQAPP VAQEALKKEL ETLTTNYQWL CTRLNGKCKT
1261 LEEVWACWHE LLSYLEKANK WLNEVEFKLK TTENIPGGAE EISEVLDSLE NLMRHSEDNP
1321 NQIRILAQTL TDGGVMDELI NEELETENSR WRELHEEAVR RQKLLEQSIQ SAQETEKSLH
1381 LIQESLTFID KQLAAYIADK VDAAQMPQEA QKIQSDLTSH EISLEEMKKH NQGKEAAQRV
1441 LSQIDVAQKK LQDVSMKERL FQKPANFELR LQESKMILDE VKMHLPALET KSVEQEVVQS
1501 QLNHCVNLYK SLSEVKSEVE MVIKTGRQIV QKKQTENPKE LDERVTALKL HYNELGAKVT
1561 ERKQQLEKCL KLSRKMRKEM NVLTEWLAAT DMELTKRSAV EGMPSNLDSE VAWGKATQKE
1621 IEKQKVHLKS ITEVGEALKT VLGKKETLVE DKLSLLNSNW IAVTSRAEEW LNLLLEYQKH
1681 METFDQNVDH ITKWIIQADT LLDESEKKKP QQKEDVLKRL KAELNDIRPK VDSTRDQAAN
1741 LMANRGDHCR KLVEPQISEL NHRFAAISHR IKTGKASIPL KELEQENSDI QKLLEPLEAE
1801 IQQGVNLKEE DENKDMNEDN EGTVKELLQR GDNLQQRITD ERKREEIKIK QQLLQTKHNA
1861 LKDLRSQRRK KALEISHQWY QYKRQADDLL KCLDDIEKKL ASLPEPRDER KIKEIDRELQ
1921 KKKEELNAVR RQAEGLSEDG AAMAVEPTQI QLSKRWREIE SKFAQFRRLN FAQIHTVREE
1981 TMMVMTEDMP LEISYVPSTY LTEITHVSQA LLEVEQLLNA PDLCAKDFED LFKQEESLKN
2041 IKDSLQQSSG RIDIIHSKKT AALQSATPVE RVKLQEALSQ LDFQWEKVNK MYKDRQGRED
2101 RSVEKWRRFH YDIKIFNQWL TEAEQFLRKT QIPENWEHAK YKWYLKELQD GIGQRQTVVR
2161 TLNATGEEII QQSSKTDASI LQEKLGSLNL RWQEVCKQLS DRKKRLEEQK NILSEFQRDL
2221 NEFVLWLEEA DNIASIPLEP GKEQQLKEKL EQVKLLVEEL PLRQGILKQL NETGGPVLVS
2281 APISPEEQDK LENKLKQTNL QWIKVSRALP EKQGEIEAQI KDLGQLEKKL EDLEEQLNHL
2341 LLWLSPIRNQ LEIYNQPNQE GPEDVQETEI AVQAKQPDVE EILSKGQHLY KEKPATQPVK
2401 RKLEDLSSEW KAVNRLLQEL RAKQPDLAPG LITIGASPTQ TVTLVTQPVV TKETAISKLE
2461 MPSSLMLEVP ALADENRAWT ELTDWLSLLD QVIKSQRVMV GDLEDINEMI IKQKATMQDL
2521 EQRRPQLEEL ITAAQNLKNK TSNQEARTII TDRIERIQNQ WDEVQEHLQN RRQQLNEMLK
2581 DSTQWLEAKE EAEQVLGQAR AKLESWKEGP YTVDAIQKKI TETKQLAKDL RQWQTNVDVA
2641 NDLALKLLRD YSADDTRKVH MITENINASW RSIHKRVSER EAALEETHRL LQQFPLDLEK
2701 FLAWLTEAET TANVLQDATR KERLLEDSKG VKELMKQWQD LQGEIEAHTD VYHNLDENSQ
2761 KILRSLEGSD DAVLLQRRLD NMNEKWSELR KKSLNIRSHL EASSDQWKRL HLSLQELLVW
2821 LQLKDDELSR QAPIGGDEPA VQKQNDVHRA FKRELKTKEP VIMSTLETVR IFLTEQPLEG
2881 LEKLYQEPRE LPPEERAQNV TRLLRKQAEE VNTEWEKLNL HSADWQRKID ETLERLQELQ
2941 EATDELDLKL RQAEVIKGSW QPVGDLLIDS LQDHLEKVKA LRGEIAPLKE NVSHVNDLAR
3001 QLTTLGIQLS PYNLSTLEDL NTRWKLLQVA VEDRVRQLHE AHRDFGPASQ HELSTSVQGP
3061 WERAISPNKV PYYINHETQT TCWDHPKMTE LYQSLADLNN VRESAYRTAM KLRRLQKALC
3121 LDLLSLSAAC DALDQHNLKQ NDQPMDILQI INCLTTIYDR LEQEHNNLVN VPLCVDMCLN
3181 WLLNVYDTGR TGRIRVLSFK TGIISLCKAH LEDKYRYLFK QVASSTGFCD QRRLGLLLHD
3241 SIQIPRQLGE VASEGGSNIE PSVRSCFQFA NNKPEIEAAL FLDWMRLEPQ SMVWLPVLHR
3301 VAAAETAKHQ AKCNICKECP IIGFRYRSLK HFNYDICQSC FFSGRVAKGH KMHYPMVEYC
3361 TPTTSGEDVR DFAKVLKNKF RTKRYFAKHP RMGYLPVQTV LEGDNMETPV TLINFWPVDS
3421 APASSPQLSH DDTHSRIEHY ASRLAEMENS NGSYLNDSIS PNESIDDEHL LIQHYCQSLN
3481 QDSPLSQPRS PAQILISLES EERGELERIL ADLEEENRNL QAEYDRLKQQ HEHKGLSPLP
3541 SPPEMMPTSP QSPRDAELIA EAKLLRQHKG RLEARMQILE DHNKQLESQL HRLRQLLEQP
3601 QAEAKVNGTT VSSPSTSLQR SDSSQPMLLR VVGSQTSDSM GEEDLLSPPQ DTSTGLEEVM
3661 EQLNNSFPSS RGRNTPGKPM REDTM.
```

In some embodiments, the gRNA targets a site within a mutant dystrophin gene. In some embodiments, the gRNA targets a dystrophin intron. In some embodiments, the gRNA targets a dystrophin exon. In some embodiments, the gRNA targets a site in a dystrophin exon that is expressed and is present in one or more of the dystrophin isoforms shown in Table 1. In embodiments, the gRNA targets a dystrophin splice site. In some embodiments, the gRNA targets a splice donor site on the dystrophin gene. In embodiments, the gRNA targets a splice acceptor site on the dystrophin gene.

TABLE 1
Dystrophin isoforms

| Sequence | Nucleic Acid | Protein | |
|---|---|---|---|
| Name | Accession No. | Accession No. | Description |
| DMD | NC_000023.11 | None | Sequence from Human X |
| Genomic | (positions | | Chromosome (at positions |
| Sequence | 31119219 to | | Xp21.2 to p21.1) from |
| | 33339609) | | Assembly GRCh38.p7 |
| | | | (GCF_000001405.33) |
| Dystrophin | NM_000109.3 | NP_000100.2 | Transcript Variant: transcript |
| Dp427c | | | Dp427c is expressed |
| isoform | | | predominantly in neurons of the |
| | | | cortex and the CA regions of |
| | | | the hippocampus. It uses a |
| | | | unique promoter/exon 1 located |
| | | | about 130 kb upstream of the |
| | | | Dp427m transcript promoter. |
| | | | The transcript includes the |
| | | | common exon 2 of transcript |
| | | | Dp427m and has a similar |
| | | | length of 14 kb. The Dp427c |
| | | | isoform contains a unique N- |
| | | | terminal MED sequence, |
| | | | instead of the |
| | | | MLWWEEVEDCY sequence |
| | | | (SEQ ID NO: 8) of isoform |
| | | | Dp427m. The remainder of |
| | | | isoform Dp427c is identical to |
| | | | isoform Dp427m. |
| Dystrophin | NM_004006.2 | NP_003997.1 | Transcript Variant: transcript |
| Dp427m | | | Dp427m encodes the main |
| isoform | | | dystrophin protein found in |
| | | | muscle. As a result of |
| | | | alternative promoter use, exon |
| | | | 1 encodes a unique N-terminal |
| | | | MLWWEEVEDCY (SEQ ID |
| | | | NO: 8) aa sequence. |
| Dystrophin | NM_004009.3 | NP_004000.1 | Transcript Variant: transcript |
| Dp427p1 | | | Dp427p1 initiates from a |
| isoform | | | unique promoter/exon 1 located |
| | | | in what corresponds to the first |
| | | | intron of transcript Dp427m. |
| | | | The transcript adds the |
| | | | common exon 2 of Dp427m |
| | | | and has a similar length (14 |
| | | | kb). The Dp427p1 isoform |
| | | | replaces the |
| | | | MLWWEEVEDCY (SEQ ID |
| | | | NO: 8) -start of Dp427m with a |
| | | | unique N-terminal MSEVSSD |
| | | | aa sequence (SEQ ID NO: 9). |
| Dystrophin | NM_004011.3 | NP_004002.2 | Transcript Variant: transcript |
| Dp260-1 | | | Dp260-1 uses exons 30-79, and |
| isoform | | | originates from a |
| | | | promoter/exon 1 sequence |
| | | | located in intron 29 of the |
| | | | dystrophin gene. As a result, |
| | | | Dp260-1 contains a 95 bp exon |
| | | | 1 encoding a unique N-terminal |
| | | | 16 aa MTEIILLIFFPAYFLN |
| | | | (SEQ ID NO: 10)-sequence that |
| | | | replaces amino acids 1-1357 of |
| | | | the full-length dystrophin |
| | | | product (Dp427m isoform). |
| Dystrophin | NM_004012.3 | NP_004003.1 | Transcript Variant: transcript |
| Dp260-2 | | | Dp260-2 uses exons 30-79, |
| isoform | | | starting from a promoter/exon 1 |
| | | | sequence located in intron 29 of |
| | | | the dystrophin gene that is |
| | | | alternatively spliced and lacks |
| | | | N-terminal amino acids 1-1357 |
| | | | of the full length dystrophin |
| | | | (Dp427m isoform). The |
| | | | Dp260-2 transcript encodes a |
| | | | unique N-terminal |
| | | | MSARKLRNLSYKK sequence |
| | | | (SEQ ID NO: 11). |
| Dystrophin | NM_004013.2 | NP_004004.1 | Transcript Variant: Dp140 |
| Dp140 | | | transcripts use exons 45-79, |
| isoform | | | starting at a promoter/exon 1 |
| | | | located in intron 44. Dp140 |
| | | | transcripts have a long (1 kb) 5′ |
| | | | UTR since translation is |
| | | | initiated in exon 51 |
| | | | (corresponding to aa 2461 of |
| | | | dystrophin). In addition to the |
| | | | alternative promoter and exon |
| | | | 1, differential splicing of exons |
| | | | 71-74 and 78 produces at least |
| | | | five Dp140 isoforms. Of these, |
| | | | this transcript (Dp140) contains |
| | | | all of the exons. |
| Dystrophin | NM_004014.2 | NP_004005.1 | Transcript Variant: transcript |
| Dp116 | | | Dp116 uses exons 56-79, |
| isoform | | | starting from a promoter/exon 1 |
| | | | within intron 55. As a result, |
| | | | the Dp116 isoform contains a |
| | | | unique N-terminal |
| | | | MLHRKTYHVK aa sequence |
| | | | (SEQ ID NO: 12), instead of aa |
| | | | 1-2739 of dystrophin. |
| | | | Differential splicing produces |
| | | | several Dp116-subtypes. The |
| | | | Dp116 isoform is also known |
| | | | as S-dystrophin or apo- |
| | | | dystrophin-2. |
| Dystrophin | NM_004015.2 | NP_004006.1 | Transcript Variant: Dp71 |
| Dp71 | | | transcripts use exons 63-79 |
| isoform | | | with a novel 80- to 100-nt exon |
| | | | containing an ATG start site for |
| | | | a new coding sequence of 17 |
| | | | nt. The short coding sequence |
| | | | is in-frame with the consecutive |
| | | | dystrophin sequence from exon |
| | | | 63. Differential splicing of |
| | | | exons 71 and 78 produces at |
| | | | least four Dp71 isoforms. Of |
| | | | these, this transcript (Dp71) |
| | | | includes both exons 71 and 78. |
| Dystrophin | NM_004016.2 | NP_004007.1 | Transcript Variant: Dp71 |
| Dp71b | | | transcripts use exons 63-79 |
| isoform | | | with a novel 80- to 100-nt exon |
| | | | containing an ATG start site for |
| | | | a new coding sequence of 17 |
| | | | nt. The short coding sequence |
| | | | is in-frame with the consecutive |
| | | | dystrophin sequence from exon |
| | | | 63. Differential splicing of |
| | | | exons 71 and 78 produces at |
| | | | least four Dp71 isoforms. Of |
| | | | these, this transcript (Dp71b) |
| | | | lacks exon 78 and encodes a |
| | | | protein with a different C- |
| | | | terminus than Dp71 and Dp71a |
| | | | isoforms. |
| Dystrophin | NM_004017.2 | NP_004008.1 | Transcript Variant: Dp71 |
| Dp71a | | | transcripts use exons 63-79 |
| isoform | | | with a novel 80- to 100-nt exon |
| | | | containing an ATG start site for |
| | | | a new coding sequence of 17 |
| | | | nt. The short coding sequence |
| | | | is in-frame with the consecutive |
| | | | dystrophin sequence from exon |
| | | | 63. Differential splicing of |
| | | | exons 71 and 78 produces at |
| | | | least four Dp71 isoforms. Of |
| | | | these, this transcript (Dp71a) |
| | | | lacks exon 71. |
| Dystrophin | NM_004018.2 | NP_004009.1 | Transcript Variant: Dp71 |
| Dp71ab | | | transcripts use exons 63-79 |
| isoform | | | with a novel 80- to 100-nt exon |
| | | | containing an ATG start site for |
| | | | a new coding sequence of 17 |
| | | | nt. The short coding sequence |
| | | | is in-frame with the consecutive |
| | | | dystrophin sequence from exon |
| | | | 63. Differential splicing of |
| | | | exons 71 and 78 produces at |
| | | | least four Dp71 isoforms. Of |
| | | | these, this transcript (Dp71ab) |
| | | | lacks both exons 71 and 78 and |
| | | | encodes a protein with a C- |
| | | | terminus like isoform Dp71b. |
| Dystrophin | NM_004019.2 | NP_004010.1 | Transcript Variant: transcript |
| Dp40 | | | Dp40 uses exons 63-70. The 5′ |
| isoform | | | UTR and encoded first 7 aa are |
| | | | identical to that in transcript |
| | | | Dp71, but the stop codon lies at |
| | | | the splice junction of the |
| | | | exon/intron 70. The 3′ UTR |
| | | | includes nt from intron 70 |
| | | | which includes an alternative |
| | | | polyadenylation site. The Dp40 |
| | | | isoform lacks the normal C- |
| | | | terminal end of full-length |
| | | | dystrophin (aa 3409-3685). |
| Dystrophin | NM_004020.3 | NP_004011.2 | Transcript Variant: Dp140 |
| Dp140c | | | transcripts use exons 45-79, |
| isoform | | | starting at a promoter/exon 1 |
| | | | located in intron 44. Dp140 |
| | | | transcripts have a long (1 kb) 5′ |
| | | | UTR since translation is |
| | | | initiated in exon 51 |
| | | | (corresponding to aa 2461 of |
| | | | dystrophin). In addition to the |
| | | | alternative promoter and exon |
| | | | 1, differential splicing of exons |
| | | | 71-74 and 78 produces at least |
| | | | five Dp140 isoforms. Of these, |
| | | | this transcript (Dp140c) lacks |
| | | | exons 71-74. |
| Dystrophin | NM_004021.2 | NP_004012.1 | Transcript Variant: Dp140 |
| Dp140b | | | transcripts use exons 45-79, |
| isoform | | | starting at a promoter/exon 1 |
| | | | located in intron 44. Dp140 |
| | | | transcripts have a long (1 kb) 5′ |
| | | | UTR since translation is |
| | | | initiated in exon 51 |
| | | | (corresponding to aa 2461 of |
| | | | dystrophin). In addition to the |
| | | | alternative promoter and exon |
| | | | 1, differential splicing of exons |
| | | | 71-74 and 78 produces at least |
| | | | five Dp140 isoforms. Of these, |
| | | | this transcript (Dp140b) lacks |
| | | | exon 78 and encodes a protein |
| | | | with a unique C-terminus. |
| Dystrophin | NM_004022.2 | NP_004013.1 | Transcript Variant: Dp140 |
| Dp140ab | | | transcripts use exons 45-79, |
| isoform | | | starting at a promoter/exon 1 |
| | | | located in intron 44. Dp140 |
| | | | transcripts have a long (1 kb) 5′ |
| | | | UTR since translation is |
| | | | initiated in exon 51 |
| | | | (corresponding to aa 2461 of |
| | | | dystrophin). In addition to the |
| | | | alternative promoter and exon |
| | | | 1, differential splicing of exons |
| | | | 71-74 and 78 produces at least |
| | | | five Dp140 isoforms. Of these, |
| | | | this transcript (Dp140ab) lacks |
| | | | exons 71 and 78 and encodes a |
| | | | protein with a unique C- |
| | | | terminus. |
| Dystrophin | NM_004023.2 | NP_004014.1 | Transcript Variant: Dp140 |
| Dp140bc | | | transcripts use exons 45-79, |
| isoform | | | starting at a promoter/exon 1 |
| | | | located in intron 44. Dp140 |
| | | | transcripts have a long (1 kb) 5′ |
| | | | UTR since translation is |
| | | | initiated in exon 51 |
| | | | (corresponding to aa 2461 of |
| | | | dystrophin). In addition to the |
| | | | alternative promoter and exon |
| | | | 1, differential splicing of exons |
| | | | 71-74 and 78 produces at least |
| | | | five Dp140 isoforms. Of these, |
| | | | this transcript (Dp140bc) lacks |
| | | | exons 71-74 and 78 and |
| | | | encodes a protein with a unique |
| | | | C-terminus. |
| Dystrophin | XM_006724469.3 | XP_006724532.1 | |
| isoform | | | |
| X2 | | | |
| Dystrophin | XM_011545467.1 | XP_011543769.1 | |
| isoform | | | |
| X5 | | | |
| Dystrophin | XM_006724473.2 | XP_006724536.1 | |
| isoform | | | |
| X6 | | | |
| Dystrophin | XM_006724475.2 | XP_006724538.1 | |
| isoform | | | |
| X8 | | | |
| Dystrophin | XM_017029328.1 | XP_016884817.1 | |
| isoform | | | |
| X4 | | | |
| Dystrophin | XM_006724468.2 | XP_006724531.1 | |
| isoform | | | |
| X1 | | | |
| Dystrophin | XM_017029331.1 | XP_016884820.1 | |
| isoform | | | |
| X13 | | | |
| Dystrophin | XM_006724470.3 | XP_006724533.1 | |
| isoform | | | |
| X3 | | | |
| Dystrophin | XM_006724474.3 | XP_006724537.1 | |
| isoform | | | |
| X7 | | | |
| Dystrophin | XM_011545468.2 | XP_011543770.1 | |
| isoform | | | |
| X9 | | | |
| Dystrophin | XM_017029330.1 | XP_016884819.1 | |
| isoform | | | |
| X11 | | | |
| Dystrophin | XM_017029329.1 | XP_016884818.1 | |
| isoform | | | |
| X10 | | | |
| Dystrophin | XM_011545469.1 | XP_011543771.1 | |
| isoform | | | |
| X12 | | | |

In embodiments, the guide RNA targets a mutant DMD exon. In some embodiments, the mutant exon is exon 53. In some embodiments, the guide RNA targets at least one of exons 1, 23, 41, 44, 46, 47, 48, 49, 50, 51, 52, 53, 54, or 55 of the dystrophin gene. In embodiments, the guide RNA targets at least one of introns 44, 45, 50, 51, 52, 53, 54, or 55 of the dystrophin gene. In preferred embodiments, the guide RNAs are designed to induce skipping and/or refraining of exon 53. In embodiments, the gRNA is targeted to a splice acceptor site of exon 53.

In some embodiments, gRNAs of the disclosure comprise a sequence that is complementary to a target sequence within a coding sequence or a non-coding sequence corresponding to the DMD gene, and, therefore, hybridize to the target sequence. In some embodiments, a nucleic acid may comprise one or more sequences encoding a gRNA. In some embodiments, a nucleic acid may comprise 1, 2, 3, 4, 5, 6, 7, 8, 9, 10, 11, 12, 13, 14, 15, 16, 17, 18, 19, 20 sequences encoding a gRNA. In some embodiments, all of the sequences encode the same gRNA. In some embodiments, all of the sequences encode different gRNAs. In some embodiments, at least 2 of the sequences encode the same gRNA, for example at least 3, 4, 5, 6, 7, 8, 9, 10, 11, 12, 13, 14, 15, 16, 17, 18, 19, or 20 of the sequences encode the same gRNA.

Improved gRNAs. In particular, the inventors have screened guide RNAs and identified a guide RNA that significantly increases productive editing while reducing non-productive editing at DMD exon 53. This sgRNA, shown labeled as sgRNA DMD Ex53 (SEQ ID NO: 13), recognizes a 5′GGTG-3′ PAM in exon 53 and generates a cut 18 base pairs upstream of the premature TGA stop codon responsible for the DMD phenotype:

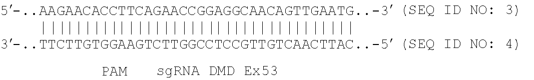

### B. Cas Nucleases

Cas Nucleases. CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcusfuriosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. The team demonstrated that they could disable one or both sites while preserving Cas9's ability to locate its target DNA. Jinek et al. (2012) combined tracrRNA and spacer RNA into a “single-guide RNA” molecule that, mixed with Cas9, can find and cut the correct DNA targets and such synthetic guide RNAs are used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Wang et al. (2013) showed that coinjection of Cas9 mRNA and sgRNAs into the germline (zygotes) generated mice with mutations. Delivery of Cas9 DNA sequences also is contemplated.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been identified as a Class II, Type V CRISPR/Cas systems containing a 1,300 amino acid protein. See also U.S. Patent Publication 2014/0068797, which is incorporated by reference in its entirety.

SpCas9 variants. In accordance with the present disclosure, there are provided variants of Staphylococcus pyogenes Cas9. These variants are particularly useful in targeting mutations in exon 53 of the DMD gene to that the open reading frame of the adjacent exons and prevent production of the dystrophin protein, representing the third most common mutation hot-spot region in the dystrophin gene.

The CRISPR/SpCas9 system from S. pyogenes has been widely used to correct DMD mutations in cells and animals. The inventors first used wild-type (WT) SpCas9 to reframe DMD exon 53 by either inserting a nucleotide or deleting two nucleotides (productive editing); however, the overall reframing efficiency was very low. This strategy resulted in many nucleotide deletions that did not reframe exon 53 of the DMD gene (nonproductive editing). Moreover, the WT spCas9 requires a 5′-NGG-3′ protospacer adjacent motif (PAM) for its RNA-guided DNA cleavage, which limited its capacity to target genomic loci.

Subsequently, the inventors engineered a CRISPR/SpCas9 variant to increase productive editing at the DMD gene loci. To this end, the engineered a SpCas9 variant (LRVQR SpCas9) by introducing 5 amino acid mutations (D1135L, G1218R, E1219V, R1335Q, T1337R) in the WT SpCas9. The LRVQR SpCas9 variant recognizes the 5′-NGT-3′ PAM for its RNA-guided DNA cleavage (as opposed to WT SpCas9 that requires a 5′-NGG-3′ protospacer). In contrast to WT SpCas9, LRVQR SpCas9 has a greatly expanded genomic targeting range. Moreover, when the LRVQR SpCas9 variant was used to target DMD exon 53, higher editing efficiency was observed. More importantly, all genome-editing events were one nucleotide insertions, making the LRVQR SpCas9-mediated reframing of DMD exon 53 highly productive, thereby significantly improveing CRISPR/SpCas9-mediated correction of Duchenne muscular dystrophy.

Additional SpCas9 variants include the following (variations enclosed in parentheses indicate amino acid mutations difference from wild-type SpCas9):
SpCas9-NG (L1111R, D1135V, G1218R, E1219F, A1322R, R1335V, T1337R)
SpCas9-SpG (D1135L, S1136W, G1218K, E1219Q, R1335Q, T1337R);
SpCas9-SpRY (A61R, L1111R, D1135L, S1136W, G1218K, E1219Q, N1317R, A1322R, R1333P, R1335Q, T1337R)
SpCas9-LKVQR (D1135L, G1218K, E1219V, R1335Q, T1337R) [LKVQR=SEQ ID NO: 14]
SpCas9-LSVQR (D1135L, G1218S, E1219V, R1335Q, T1337R) [LSVQR=SEQ ID NO: 15]
SpCas9-LWKVQR (D1135L, S1136W, G1218K, E1219V, R1335Q, T1337R) [LWKVQR=SEQ ID NO: 16]
SpCas9-LWSVQR (D1135L, S1136W, G1218S, E1219V, R1335Q, T1337R) [LWSVQR=SEQ ID NO: 17]
SpCas9-LWRVQR (D1135L, S1136W, G1218R, E1219V, R1335Q, T1337R) [LWRVQR=SEQ ID NO: 18]
SpCas9-LWKSQR (D1135L, 51136W, G1218K, E1219S, R1335Q, T1337R) [LWKSQR=SEQ ID NO: 19]
SpCas9-LWSSQR (D1135L, S1136W, G1218S, E1219S, R1335Q, T1337R) [LWSSQR=SEQ ID NO: 20]
SpCas9-LWRSQR (D1135L, S1136W, G1218R, E1219S, R1335Q, T1337R) [LWRSQR=SEQ ID NO: 21]
SpCas9-LKVQR (D1135L, G1218K, E1219V, R1335Q, T1337R) [LKVQR=SEQ ID NO: 22]
SpCas9-LSVQR (D1135L, G1218S, E1219V, R1335Q, T1337R) [LSVQR=SEQ ID NO: 23]
SpCas9-LWKVQR (D1135L, S1136W, G1218K, E1219V, R1335Q, T1337R) [LWKVQR=SEQ ID NO: 24]
SpCas9-LWSVQR (D1135L, S1136W, G1218S, E1219V, R1335Q, T1337R) [LWSVQR=SEQ ID NO: 25]
SpCas9-LWRVQR (D1135L, S1136W, G1218R, E1219V, R1335Q, T1337R) [LWRVQR=SEQ ID NO: 26]
SpCas9-LWKSQR (D1135L, S1136W, G1218K, E1219S, R1335Q, T1337R) [LWKSQR=SEQ ID NO: 27]
SpCas9-LWSSQR (D1135L, S1136W, G1218S, E1219S, R1335Q, T1337R) [LWSSQR=SEQ ID NO: 28]
SpCas9-LWRSQR (D1135L, S1136W, G1218R, E1219S, R1335Q, T1337R) [LWRSQR=SEQ ID NO: 29]

### C. CRISPR-Mediated Gene Editing

The first step in editing the DMD gene using CRISPR/Cas9 is to identify the genomic target sequence. The genomic target for the gRNAs of the disclosure can be any ˜24 nucleotide DNA sequence, provided that the sequence is unique compared to the rest of the genome. In some embodiments, the genomic target sequence corresponds to a sequence within exon 51, exon 45, exon 44, exon 53, exon 46, exon 52, exon 50, exon 43, exon 6, exon 7, exon 8, and/or exon 55 of the human dystrophin gene. In some embodiments, the genomic target sequence is a 5′ or 3′ splice site of exon 51, exon 45, exon 44, exon 53, exon 46, exon 52, exon 50, exon 43, exon 6, exon 7, exon 8, and/or exon 55 of the human dystrophin gene. In some embodiments, the genomic target sequence corresponds to a sequence within an intron immediately upstream or downstream of exon 51, exon 45, exon 44, exon 53, exon 46, exon 52, exon 50, exon 43, exon 6, exon 7, exon 8, and/or exon 55 of the human dystrophin gene. Exemplary genomic target sequences can be found in Tables 6, 8, 10, 12, and 14.

The next step in editing the DMD gene is to identify all Protospacer Adjacent Motif (PAM) sequences within the genetic region to be targeted. The target sequence must be immediately upstream of a PAM. Once all possible PAM sequences and putative target sites have been identified, the next step is to choose which site is likely to result in the most efficient on-target cleavage. The gRNA targeting sequence needs to match the target sequence, and the gRNA targeting sequence must not match additional sites within the genome. In preferred embodiments, the gRNA targeting sequence has perfect homology to the target with no homology elsewhere in the genome. In some embodiments, a given gRNA targeting sequence will have additional sites throughout the genome where partial homology exists. These sites are called “off-targets” and should be considered when designing a gRNA. In general, off-target sites are not cleaved as efficiently when mismatches occur near the PAM sequence, so gRNAs with no homology or those with mismatches close to the PAM sequence will have the highest specificity. In addition to “off-target activity”, factors that maximize cleavage of the desired target sequence (“on-target activity”) must be considered. It is known to those of skill in the art that two gRNA targeting sequences, each having 100% homology to the target DNA may not result in equivalent cleavage efficiency. In fact, cleavage efficiency may increase or decrease depending upon the specific nucleotides within the selected target sequence. Close examination of predicted on-target and off-target activity of each potential gRNA targeting sequence is necessary to design the best gRNA. Several gRNA design programs have been developed that are capable of locating potential PAM and target sequences and ranking the associated gRNAs based on their predicted on-target and off-target activity (e.g. CRISPRdirect, available at the world-wide-web at crispr.dbcls.jp).

The next step is to synthesize and clone desired gRNAs. Targeting oligos can be synthesized, annealed, and inserted into plasmids containing the gRNA scaffold using standard restriction-ligation cloning. However, the exact cloning strategy will depend on the gRNA vector that is chosen.

Each gRNA should then be validated in one or more target cell lines. For example, after the Cas9 and the gRNA are delivered to the cell, the genomic target region may be amplified using PCR and sequenced according to methods known to those of skill in the art.

In some embodiments, gene editing may be performed in vitro or ex vivo. In some embodiments, cells are contacted in vitro or ex vivo with a Cas9 and a gRNA that targets a dystrophin splice site. In some embodiments, the cells are contacted with one or more nucleic acids encoding the Cas9 and the guide RNA. In some embodiments, the one or more nucleic acids are introduced into the cells using, for example, lipofection or electroporation. Gene editing may also be performed in zygotes. In embodiments, zygotes may be injected with one or more nucleic acids encoding Cas9 and a gRNA that targets a dystrophin splice site. The zygotes may subsequently be injected into a host.

In some embodiments, the Cas9 is provided on a vector. In some embodiments, the Cas9 sequence is codon optimized for expression in human cells or mouse cells. In some embodiments, the vector further contains a sequence encoding a fluorescent protein, such as GFP, which allows Cas 9-expressing cells to be sorted using fluorescence activated cell sorting (FACS). In some embodiments, the vector is a viral vector such as an adeno-associated viral vector.

In some embodiments, the gRNA is provided on a vector. In some embodiments, the vector is a viral vector such as an adeno-associated viral vector. In embodiments, the Cas9 and the guide RNA are provided on the same vector. In embodiments, the Cas9 and the guide RNA are provided on different vectors.

In some embodiments, the cells are additionally contacted with a single-stranded DMD oligonucleotide to effect homology directed repair. In some embodiments, small INDELs restore the protein reading frame of dystrophin (“reframing” strategy). When the reframing strategy is used, the cells may be contacted with a single gRNA. In embodiments, a splice donor or splice acceptor site is disrupted, which results in exon skipping and restoration of the protein reading frame (“exon skipping” strategy). When the exon skipping strategy is used, the cells may be contacted with two or more gRNAs.

Efficiency of in vitro or ex vivo Cas9-mediated DNA cleavage may be assessed using techniques known to those of skill in the art, such as the T7 E1 assay. Restoration of DMD expression may be confirmed using techniques known to those of skill in the art, such as RT-PCR, western blotting, and immunocytochemistry.

In some embodiments, in vitro or ex vivo gene editing is performed in a muscle or satellite cell. In some embodiments, gene editing is performed in iPSC or iCM cells. In embodiments, the iPSC cells are differentiated after gene editing. For example, the iPSC cells may be differentiated into a muscle cell or a satellite cell after editing. In embodiments, the iPSC cells are differentiated into cardiac muscle cells, skeletal muscle cells, or smooth muscle cells. In embodiments, the iPSC cells are differentiated into cardiomyocytes. iPSC cells may be induced to differentiate according to methods known to those of skill in the art.

In some embodiments, contacting the cell with the Cas9 and the gRNA restores dystrophin expression. In embodiments, cells which have been edited in vitro or ex vivo, or cells derived therefrom, show levels of dystrophin protein that is comparable to wild-type cells. In embodiments, the edited cells, or cells derived therefrom, express dystrophin at a level that is 50%, 60%, 70%, 80%, 90%, 95% or any percentage in between of wild-type dystrophin expression levels. In embodiments, the cells which have been edited in vitro or ex vivo, or cells derived therefrom, have a mitochondrial number that is comparable to that of wild-type cells. In embodiments the edited cells, or cells derived therefrom, have 50%, 60%, 70%, 80%, 90%, 95% or any percentage in between as many mitochondria as wild-type cells. In embodiments, the edited cells, or cells derived therefrom, show an increase in oxygen consumption rate (OCR) compared to non-edited cells at baseline.

### II. NUCLEIC ACID EXPRESSION VECTORS

As discussed above, in certain embodiments, expression cassettes are employed to express a transcription factor product, either for subsequent purification and delivery to a cell/subject, or for use directly in a genetic-based delivery approach. Provided herein are expression vectors which contain one or more nucleic acids encoding Cas9 and at least one DMD guide RNA that targets a dystrophin splice site. In some embodiments, a nucleic acid encoding Cas9 and a nucleic acid encoding at least one guide RNA are provided on the same vector. In further embodiments, a nucleic acid encoding Cas9 and a nucleic acid encoding least one guide RNA are provided on separate vectors.

Expression requires that appropriate signals be provided in the vectors and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

### A. Regulatory Elements

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

RNA Polymerase and Pol III Promoters. In eukaryotes, RNA polymerase III (also called Pol III) transcribes DNA to synthesize ribosomal 5S rRNA, tRNA and other small RNAs. The genes transcribed by RNA Pol III fall in the category of “housekeeping” genes whose expression is required in all cell types and most environmental conditions. Therefore, the regulation of Pol III transcription is primarily tied to the regulation of cell growth and the cell cycle, thus requiring fewer regulatory proteins than RNA polymerase II. Under stress conditions however, the protein Maf1 represses Pol III activity.

In the process of transcription (by any polymerase) there are three main stages: (i) initiation, requiring construction of the RNA polymerase complex on the gene's promoter; (ii) elongation, the synthesis of the RNA transcript; and (iii) termination, the finishing of RNA transcription and disassembly of the RNA polymerase complex.

Promoters under the control of RNA Pol III include those for ribosomal 5S rRNA, tRNA and few other small RNAs such as U6 spliceosomal RNA, RNase P and RNase MRP RNA, 7SL RNA (the RNA component of the signal recognition particles), Vault RNAs, Y RNA, SINEs (short interspersed repetitive elements), 7SK RNA, two microRNAs, several small nucleolar RNAs and several few regulatory antisense RNAs.

Additional Promoters and Elements. In some embodiments, the Cas9 constructs of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promoters such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2κb, HSP70, proliferin, tumor necrosis factor, and/or thyroid stimulating hormone α gene. In some embodiments, the inducer is phorbol ester (TFA), heavy metals, glucocorticoids, poly(rI)x, poly(rc), E1A, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Of particular interest are muscle specific promoters. These include the myosin light chain-2 promoter, the α-actin promoter, the troponin 1 promoter; the Na⁺/Ca²⁺ exchanger promoter, the dystrophin promoter, the α7 integrin promoter, the brain natriuretic peptide promoter and the αB-crystallin/small heat shock protein promoter, α-myosin heavy chain promoter and the ANF promoter.

In some embodiments, the muscle specific promoter is the CK8 promoter. The CK8 promoter has the following sequence (SEQ ID NO: 30):

```
    1 CTAGACTAGC ATGCTGCCCA TGTAAGGAGG CAAGGCCTGG GGACACCCGA GATGCCTGGT
  61 TATAATTAAC CCAGACATGT GGCTGCCCCC CCCCCCCCAA CACCTGCTGC CTCTAAAAAT
121 AACCCTGCAT GCCATGTTCC CGGCGAAGGG CCAGCTGTCC CCCGCCAGCT AGACTCAGCA
181 CTTAGTTTAG GAACCAGTGA GCAAGTCAGC CCTTGGGGCA GCCCATACAA GGCCATGGGG
241 CTGGGCAAGC TGCACGCCTG GGTCCGGGGT GGGCACGGTG CCCGGGCAAC GAGCTGAAAG
301 CTCATCTGCT CTCAGGGGCC CCTCCCTGGG GACAGCCCCT CCTGGCTAGT CACACCCTGT
361 AGGCTCCTCT ATATAACCCA GGGGCACAGG GGCTGCCCTC ATTCTACCAC CACCTCCACA
421 GCACAGACAG ACACTCAGGA GCCAGCCAGC.
```

In some embodiments, the muscle-cell cell specific promoter is a variant of the CK8 promoter, called CK8e. The CK8e promoter has the following sequence (SEQ ID NO: 31):

```
    1 TGCCCATGTA AGGAGGCAAG GCCTGGGGAC ACCCGAGATG CCTGGTTATA ATTAACCCAG
  61 ACATGTGGCT GCCCCCCCCC CCCCAACACC TGCTGCCTCT AAAAATAACC CTGCATGCCA
121 TGTTCCCGGC GAAGGGCCAG CTGTCCCCCG CCAGCTAGAC TCAGCACTTA GTTTAGGAAC
181 CAGTGAGCAA GTCAGCCCTT GGGGCAGCCC ATACAAGGCC ATGGGGCTGG GCAAGCTGCA
241 CGCCTGGGTC CGGGGTGGGC ACGGTGCCCG GGCAACGAGC TGAAAGCTCA TCTGCTCTCA
301 GGGGCCCCTC CCTGGGGACA GCCCCTCCTG GCTAGTCACA CCCTGTAGGC TCCTCTATAT
361 AACCCAGGGG CACAGGGGCT GCCCTCATTC TACCACCACC TCCACAGCAC AGACAGACAC
421 TCAGGAGCCA GCCAGC.
```

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

### B. Self-Cleaving Peptides

In some embodiments of self-cleaving peptides of the disclosure, the self-cleaving peptide is a 2A peptide. In some embodiments, a 2A-like self-cleaving domain from the insect virus Thosea asigna (TaV 2A peptide) (SEQ ID NO: 32, EGRGSLLTCGDVEENPGP) is used. These 2A-like domains have been shown to function across eukaryotes and cause cleavage of amino acids to occur co-translationally within the 2A-like peptide domain. Therefore, inclusion of TaV 2A peptide allows the expression of multiple proteins from a single mRNA transcript. Importantly, the domain of TaV when tested in eukaryotic systems has shown greater than 99% cleavage activity. Other acceptable 2A-like peptides include, but are not limited to, equine rhinitis A virus (ERAV) 2A peptide (SEQ ID NO: 33; QCTNYALLKLAGDVESNPGP), porcine teschovirus-1 (PTV1) 2A peptide (SEQ ID NO: 34; ATNFSLLKQAGDVEENPGP) and foot and mouth disease virus (FMDV) 2A peptide (SEQ ID NO: 35; PVKQLLNFDLLKLAGDVESNPGP) or modified versions thereof. In some embodiments, the 2A peptide is used to express a reporter and a Cas9 simultaneously. The reporter may be, for example, GFP.

Other self-cleaving peptides that may be used include but are not limited to nuclear inclusion protein a (Nia) protease, a P1 protease, a 3C protease, an L protease, a 3C-like protease, or modified versions thereof.

### III. THERAPEUTIC COMPOSITIONS

### A. AAV-Cas9 Vectors

In some embodiments, a Cas9 may be packaged into an AAV vector. In some embodiments, the AAV vector is a wild-type AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11, AAVrh74, AAVrh10 or any combination thereof.

Exemplary AAV-Cas9 vectors contain two ITR (inverted terminal repeat) sequences which flank a central sequence region comprising the Cas9 sequence. In some embodiments, the ITRs are isolated or derived from an AAV vector of serotype AAV1, AAV2, AAV3, AAV4, AAV5, AAV6, AAV7, AAV8, AAV9, AAV10, AAV11 AAVrh74, AAVrh10 or any combination thereof. In some embodiments, the ITRs comprise or consist of full-length and/or wild-type sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wild-type sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV-Cas9 vector may contain one or more nuclear localization signals (NLS). In some embodiments, the AAV-Cas9 vector contains 1, 2, 3, 4, or 5 nuclear localization signals. Exemplary NLS include the c-myc NLS, the SV40 NLS, the hnRNPAI M9 NLS, the nucleoplasmin NLS, the sequence RMRKFKNKGKDTAELRRRRVEVSVELRKAKKDEQILKRRNV (SEQ ID NO: 36) of the IBB domain from importin-alpha, the sequences VSRKRPRP (SEQ ID NO: 37) and PPKKARED (SEQ ID NO: 38) of the myoma T protein, the sequence PQPKKKPL (SEQ ID NO: 39) of human p53, the sequence SALIKKKKKMAP (SEQ ID NO: 40) of mouse c-abl IV, the sequences DRLRR (SEQ ID NO: 41) and KQKKRK (SEQ ID NO: 42) of the influenza virus NS1, the sequence RKLKKKIKKL (SEQ ID NO: 43) of the Hepatitis virus delta antigen and the sequence REKKKFLKRR (SEQ ID NO: 44) of the mouse Mxl protein. Further acceptable nuclear localization signals includebipartite nuclear localization sequences such as the sequence KRKGDEVDGVDEVAKKKSKK (SEQ ID NO: 45 of the human poly(ADP-ribose) polymerase or the sequence RKCLQAGMNLEARKTKK (SEQ ID NO: 46) of the steroid hormone receptors (human) glucocorticoid.

In some embodiments, the AAV-Cas9 vector may comprise additional elements to facilitate packaging of the vector and expression of the Cas9. In some embodiments, the AAV-Cas9 vector may comprise a polyA sequence. In some embodiments, the polyA sequence may be a mini-polyA sequence. In some embodiments, the AAV-Cas9 vector may comprise a transposable element. In some embodiments, the AAV-Cas9 vector may comprise a regulator element. In some embodiments, the regulator element is an activator or a repressor.

In some embodiments, the AAV-Cas9 may contain one or more promoters. In some embodiments, the one or more promoters drive expression of the Cas9. In some embodiments, the one or more promoters are muscle-specific promoters. Exemplary muscle-specific promoters include myosin light chain-2 promoter, the α-actin promoter, the troponin 1 promoter, the Na⁺/Ca²⁺ exchanger promoter, the dystrophin promoter, the α7 integrin promoter, the brain natriuretic peptide promoter, the αB-crystallin/small heat shock protein promoter, α-myosin heavy chain promoter, the ANF promoter, the CK8 promoter and the CK8e promoter.

In some embodiments, the AAV-Cas9 vector may be optimized for production in yeast, bacteria, insect cells, or mammalian cells. In some embodiments, the AAV-Cas9 vector may be optimized for expression in human cells. In some embodiments, the AAV-Cas9 vector may be optimized for expression in a bacculovirus expression system.

In some embodiments, the construct comprises or consists of a promoter and a nuclease. In some embodiments, the construct comprises or consists of a CK8e promoter and a Cas9 nuclease. In some embodiments, the construct comprises or consists of a CK8e promoter and a Cas9 nuclease as described herein. In some embodiments, the construct comprising a promoter and a nuclease further comprises at least two inverted terminal repeat (ITR) sequences. In some embodiments, the construct comprising a promoter and a nuclease further comprises at least two ITR sequences from isolated or derived from an AAV of serotype 2 (AAV2). In some embodiments, the construct comprising a promoter and a nuclease further comprises at least two ITR sequences each comprising or consisting of a nucleotide sequence of GGCCACTCCCTCTCTGCGCGCTCGCTCGCTCACTGAGGCCGGGCGACCAAAGGTC GCCCGACGCCCGGGCTTTGCCCGGGCGGCCTCAGTGAGCGAGCGAGCGCGCAGA GAGGGA (SEQ ID NO: 47). In some embodiments, the construct comprising a promoter and a nuclease further comprises at least two ITR sequences, wherein the first ITR sequence comprises or consists of a nucleotide sequence of CCTGCAGGCAGCTGCGCGCTCGCTCGCTCACTGAGGCCGCCCGGGCAAAGCCCG GGCGTCGGGCGACCTTTGGTCGCCCGGCCTCAGTGAGCGAGCGAGCGCGCAGAG AGGGAGTGGCCAACTCCATCACTAGGGGTTCCT (SEQ ID NO: 48) and the second ITR sequence comprises or consists of a nucleotide sequence of AGGAACCCCTAGTGATGGAGTTGGCCACTCCCTCTCTGCGCGCTCGCTCGCTCAC TGAGGCCGGGCGACCAAAGGTCGCCCGACGCCCGGGCTTTGCCCGGGCGGCCTC AGTGAGCGAGCGAGCGCGCAGCTGCCTGCAGG (SEQ ID NO: 49). In some embodiments, the construct comprises or consists of, from 5′ to 3′ a first ITR, a sequence encoding a promoter, a sequence encoding a nuclease and a second ITR. In some embodiments, the construct comprises or consists of, from 5′ to 3′ a first AAV2 ITR, a sequence encoding a CK8e promoter, a sequence encoding a SpCas9 nuclease as described herein and a second AAV2 ITR. In some embodiments, the construct comprising or consisting of, from 5′ to 3′ a first ITR, a sequence encoding a promoter, a sequence encoding a nuclease and a second ITR, further comprises a poly A sequence. In some embodiments, the polyA sequence comprises or consists of a minipolyA sequence. Exemplary minipolyA sequences of the disclosure comprise or consist of a nucleotide sequence of TAGCAATAAAGGATCGTTTATTTTCATTGGAAGCGTGTGTTGGTTTTTTGATCAGG CGCG (SEQ ID NO: 50). In some embodiments, the construct comprises or consists of, from 5′ to 3′ a first ITR, a sequence encoding a promoter, a sequence encoding a nuclease, a poly A sequence and a second ITR. In some embodiments, the construct comprises or consists of, from 5′ to 3′ a first ITR, a sequence encoding a promoter, a sequence encoding a nuclease, a minipoly A sequence and a second ITR. In some embodiments, the construct comprises or consists of, from 5′ to 3′ a first AAV2 ITR, a sequence encoding a CK8e promoter, a sequence encoding a SpCas9 as described herein, a minipoly A sequence and a second AAV2 ITR. In some embodiments, the construct comprising, from 5′ to 3′ a first ITR, a sequence encoding a promoter, a sequence encoding a nuclease, a poly A sequence and a second ITR, further comprises at least one nuclear localization signal. In some embodiments, the construct comprising, from 5′ to 3′ a first ITR, a sequence encoding a promoter, a sequence encoding a nuclease, a poly A sequence and a second ITR, further comprises at least two nuclear localization signals. Exemplary nuclear localization signals of the disclosure comprise or consist of a nucleotide sequence of AAGCGTCCTGCTGCTACTAAGAAAGCTGGTCAAGCTAAGAAAAAGAAA (SEQ ID NO: 51), or a nucleotide sequence of ATGGCCCCAAAGAAGAAGCGGAAGGTCGGTATCCACGGAGTCCCAGCAGCC (SEQ ID NO: 52). In some embodiments, the construct comprises or consists of, from 5′ to 3′ a first ITR, a sequence encoding a promoter, a sequence encoding a first nuclear localization signal, a sequence encoding a nuclease, a poly A sequence and a second ITR. In some embodiments, the construct comprises or consists of, from 5′ to 3′ a first ITR, a sequence encoding a promoter, a sequence encoding a first nuclear localization signal, a sequence encoding a nuclease, a sequence encoding a second nuclear localization signal, a poly A sequence and a second ITR. In some embodiments, the construct comprising, from 5′ to 3′ a first ITR, a sequence encoding a promoter, a sequence encoding a first nuclear localization signal, a sequence encoding a nuclease, a sequence encoding a second nuclear localization signal, a poly A sequence and a second ITR, further comprises a stop codon. The stop codon may have a sequence of TAG, TAA, or TGA. In some embodiments, the construct comprises or consists of, from 5′ to 3′ a first ITR, a sequence encoding a promoter, a sequence encoding a first nuclear localization signal, a sequence encoding a nuclease, a sequence encoding a second nuclear localization signal, a stop codon, a poly A sequence and a second ITR. In some embodiments, the construct comprising or consisting of, from 5′ to 3′ a first ITR, a sequence encoding a promoter, a sequence encoding a first nuclear localization signal, a sequence encoding a nuclease, a sequence encoding a second nuclear localization signal, a stop codon, a poly A sequence and a second ITR, further comprises transposable element inverted repeats. Exemplary transposable element inverted repeats of the disclosure comprise or consist of a nucleotide sequence of TGTGGGCGGACAAAATAGTTGGGAACTGGGAGGGGTGGAAATGGAGTTTTTAAG GATTATTTAGGGAAGAGTGACAAAATAGATGGGAACTGGGTGTAGCGTCGTAAG CTAATACGAAAATTAAAAATGACAAAATAGTTTGGAACTAGATTTCACTTATCTG GTT (SEQ ID NO: 53) and/or a nucleotide sequence of GAATATAGTCTTTACCATGCCCTTGGCCACGCCCCTCTTTAATACGACGGGCAAT TTGCACTTCAGAAAATGAAGAGTTTGCTTTAGCCATAACAAAAGTCCAGTATGCT TTTTCACAGCATAACTGGACTGATTTCAGTTTACAACTATTCTGTCTAGTTTAAGA CTTTATTGTCATAGTTTAGATCTATTTTGTTCAGTTTAAGACTTTATTGTCCGCCCA CA (SEQ ID NO: 54). In some embodiments, the construct comprises or consists of, from 5′ to 3′ a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, and a second transposable element inverted repeat. In some embodiments, the construct comprising or consisting of, from 5′ to 3′, a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, and a second transposable element inverted repeat, further comprises a regulatory sequence. Exemplary regulatory sequences of the disclosure comprise or consist of a nucleotide sequence of CATGCAAGCTGTAGCCAACCACTAGAACTATAGCTAGAGTCCTGGGCGAACAAA CGATGCTCGCCTTCCAGAAAACCGAGGATGCGAACCACTTCATCCGGGGTCAGC ACCACCGGCAAGCGCCGCGACGGCCGAGGTCTTCCGATCTCCTGAAGCCAGGGC AGATCCGTGCACAGCACCTTGCCGTAGAAGAACAGCAAGGCCGCCAATGCCTGA CGATGCGTGGAGACCGAAACCTTGCGCTCGTTCGCCAGCCAGGACAGAAATGCC TCGACTTCGCTGCTGCCCAAGGTTGCCGGGTGACGCACACCGTGGAAACGGATG AAGGCACGAACCCAGTTGACATAAGCCTGTTCGGTTCGTAAACTGTAATGCAAG TAGCGTATGCGCTCACGCAACTGGTCCAGAACCTTGACCGAACGCAGCGGTGGT AACGGCGCAGTGGCGGTTTTCATGGCTTGTTATGACTGTTTTTTTGTACAGTCTAT GCCTCGGGCATCCAAGCAGCAAGCGCGTTACGCCGTGGGTCGATGTTTGATGTTA TGGAGCAGCAACGATGTTACGCAGCAGCAACGATGTTACGCAGCAGGGCAGTCG CCCTAAAACAAAGTTAGGTGGCTCAAGTATGGGCATCATTCGCACATGTAGGCTC GGCCCTGACCAAGTCAAATCCATGCGGGCTGCTCTTGATCTTTTCGGTCGTGAGT TCGGAGACGTAGCCACCTACTCCCAACATCAGCCGGACTCCGATTACCTCGGGA ACTTGCTCCGTAGTAAGACATTCATCGCGCTTGCTGCCTTCGACCAAGAAGCGGT TGTTGGCGCTCTCGCGGCTTACGTTCTGCCCAAGTTTGAGCAGCCGCGTAGTGAG ATCTATATCTATGATCTCGCAGTCTCCGGCGAGCACCGGAGGCAGGGCATTGCCA CCGCGCTCATCAATCTCCTCAAGCATGAGGCCAACGCGCTTGGTGCTTATGTGAT CTACGTGCAAGCAGATTACGGTGACGATCCCGCAGTGGCTCTCTATACAAAGTTG GGCATACGGGAAGAAGTGATGCACTTTGATATCGACCCAAGTACCGCCACCTAA CAATTCGTTCAAGCCGAGATCGGCTTCCCGGCCGCGGAGTTGTTCGGTAAATTGT CACAACGCCG (SEQ ID NO: 55). In some embodiments, the construct comprises or consists of, from 5′ to 3′ a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, a regulatory sequence and a second transposable element inverted repeat. In some embodiments, the construct may further comprise one or more spacer sequences. Exemplary spacer sequences of the disclosure have length from 1-1500 nucleotides, inclusive of all ranges therebetween. In some embodiments, the spacer sequences may be located either 5′ to or 3′ to an ITR, a promoter, a nuclear localization sequence, a nuclease, a stop codon, a polyA sequence, a transposable element inverted repeat, and/or a regulator element.

### B. AAV-sgRNA Vectors

In some embodiments, at least a first sequence encoding a gRNA and a second sequence encoding a gRNA may be packaged into an AAV vector. In some embodiments, at least a first sequence encoding a gRNA, a second sequence encoding a gRNA, and a third sequence encoding a gRNA may be packaged into an AAV vector. In some embodiments, at least a first sequence encoding a gRNA, a second sequence encoding a gRNA, a third sequence encoding a gRNA, and a fourth sequence encoding a gRNA may be packaged into an AAV vector. In some embodiments, at least a first sequence encoding a gRNA, a second sequence encoding a gRNA, a third sequence encoding a gRNA, a fourth sequence encoding a gRNA, and a fifth sequence encoding a gRNA may be packaged into an AAV vector. In some embodiments, a plurality of sequences encoding a gRNA are packaged into an AAV vector. For example, 1, 2, 3, 4, 5, 6, 7, 8, 9, 10, 11, 12, 13, 14, 15, 16, 17, 18, 19, or 20 sequences encoding a gRNA may be packaged into an AAV vector. In some embodiments, each sequence encoding a gRNA is different. In some embodiments, at least 1, 2, 3, 4, 5, 6, 7, 8, 9, 10, 11, 12, 13, 14, 15, 16, 17, 18, 19, or 20 of the sequences encoding a gRNA are the same. In some embodiments, all of the sequence encoding a gRNA are the same.

In some embodiments, the AAV vector is a wild-type AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11, AAVrh74, AAVrh10 or any combination thereof.

Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11, AAVrh74, AAVrh10 or any combination thereof. In some embodiments, the ITRs are isolated or derived from an AAV vector of a first serotype and a sequence encoding a capsid protein of the AAV-sgRNA vector is isolated or derived from an AAV vector of a second serotype. In some embodiments, the first serotype and the second serotype are the same. In some embodiments, the first serotype and the second serotype are not the same. In some embodiments, the first serotype is AAV1, AAV2, AAV3, AAV4, AAV5, AAV6, AAV7, AAV8, AAV9, AAV10, AAV11, AAVrh74, or AAVrh10. In some embodiments, the second serotype is AAV1, AAV2, AAV3, AAV4, AAV5, AAV6, AAV7, AAV8, AAV9, AAV10, AAV11, AAVrh74, or AAVrh10. In some embodiments, the first serotype is AAV2 and the second serotype is AAV1, AAV2, AAV3, AAV4, AAV5, AAV6, AAV7, AAV8, AAV9, AAV10, AAV11, AAVrh74, or AAVrh10. In some embodiments, the first serotype is AAV2 and the second serotype is AAV9.

Exemplary AAV-sgRNA vectors contain two ITR (inverted terminal repeat) sequences which flank a central sequence region comprising the gRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11, AAVrh74, AAVrh10 or any combination thereof. In some embodiments, a first ITR is isolated or derived from an AAV vector of a first serotype, a second ITR is isolated or derived from an AAV vector of a second serotype and a sequence encoding a capsid protein of the AAV-sgRNA vector is isolated or derived from an AAV vector of a third serotype. In some embodiments, the first serotype and the second serotype are the same. In some embodiments, the first serotype and the second serotype are not the same. In some embodiments, the first serotype, the second serotype, and the third serotype are the same. In some embodiments, the first serotype, the second serotype, and the third serotype are not the same. In some embodiments, the first serotype is AAV1, AAV2, AAV3, AAV4, AAV5, AAV6, AAV7, AAV8, AAV9, AAV10, AAV11, AAVrh74, or AAVrh10. In some embodiments, the second serotype is AAV1, AAV2, AAV3, AAV4, AAV5, AAV6, AAV7, AAV8, AAV9, AAV10, AAV11, AAVrh74, or AAVrh10. In some embodiments, the third serotype is AAV1, AAV2, AAV3, AAV4, AAV5, AAV6, AAV7, AAV8, AAV9, AAV10, AAV11, AAVrh74, or AAVrh10. In some embodiments, the first serotype is AAV2, the second serotype is AAV4 and the third serotype is AAV1, AAV2, AAV3, AAV4, AAV5, AAV6, AAV7, AAV8, AAV9, AAV10, AAV11, AAVrh74, or AAVrh10. In some embodiments, the first serotype is AAV2, the second serotype is AAV4 and the third serotype is AAV9. Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11, AAVrh74, or AAVrh10 or any combination thereof. In some embodiments, the ITRs comprise or consist of full-length and/or wild-type sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wild-type sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV-sgRNA vector may comprise additional elements to facilitate packaging of the vector and expression of the sgRNA. In some embodiments, the AAV-sgRNA vector may comprise a transposable element. In some embodiments, the AAV-sgRNA vector may comprise a regulatory element. In some embodiments, the regulatory element comprises an activator or a repressor. In some embodiments, the AAV-sgRNA sequence may comprise a non-functional or “stuffer” sequence. Exemplary stuffer sequences of the disclosure may have some (a non-zero percentage of) identity or homology to a genomic sequence of a mammal (including a human). Alternatively, exemplary stuffer sequences of the disclosure may have no identify or homology to a genomic sequence of a mammal (including a human). Exemplary stuffer sequences of the disclosure may comprise or consist of naturally occurring non-coding sequences or sequences that are neither transcribed nor translated following administration of the AAV vector to a subject.

In some embodiments, the AAV-sgRNA vector may be optimized for production in yeast, bacteria, insect cells, or mammalian cells. In some embodiments, the AAV-sgRNA vector may be optimized for expression in human cells. In some embodiments, the AAV-Cas9 vector may be optimized for expression in a bacculovirus expression system.

In some embodiments, the AAV-sgRNA vector comprises at least one promoter. In some embodiments, the AAV-sgRNA vector comprises at least two promoters. In some embodiments, the AAV-sgRNA vector comprises at least three promoters. In some embodiments, the AAV-sgRNA vector comprises at least four promoters. In some embodiments, the AAV-sgRNA vector comprises at least five promoters. Exemplary promoters include, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), Duchenne muscular dystrophy, SV40, polyoma, retroviruses, papilloma virus, hepatitis B virus, human immunodeficiency virus, cytomegalovirus (CMV), and gibbon ape leukemia virus. Further exemplary promoters include the U6 promoter, the H1 promoter, and the 7SK promoter.

In some embodiments, the AAV vector comprises a first sequence encoding a gRNA and a second sequence encoding a gRNA, a first promoter drives expression of the first sequence encoding a gRNA and a second promoter drives expression of the second sequence encoding a gRNA. In some embodiments, the first and second promoters are the same. In some embodiments, the first and second promoters are different. In some embodiments, the first and second promoters are selected from the H1 promoter, the U6 promoter, and the 7SK promoter. In some embodiments, the first sequence encoding a gRNA and the second sequence encoding a gRNA are identical. In some embodiments, the first sequence encoding a gRNA and the second sequence encoding a gRNA are not identical.

In some embodiments, the AAV vector comprises a first sequence encoding a gRNA, a second sequence encoding a gRNA, and a third sequence encoding a gRNA, a first promoter drives expression of the first sequence encoding a gRNA, a second promoter drives expression of the second sequence encoding a gRNA, and a third promoter drives expression of a third sequence encoding a gRNA. In some embodiments, at least two of the first, second, and third promoters are the same. In some embodiments, each of the first, second, and third promoters are different. In some embodiments, the first, second, and third promoters are selected from the H1 promoter, the U6 promoter, and the 7SK promoter. In some embodiments, the first promoter is the U6 promoter. In some embodiments, the second promoter is the H1 promoter. In some embodiments, the third promoter is the 7SK promoter. In some embodiments, the first promoter is the U6 promoter, the second promoter is the H1 promoter, and the third promoter is the 7SK promoter. In some embodiments, the first sequence encoding a gRNA, the second sequence encoding a gRNA, and the third sequence encoding a gRNA are identical. In some embodiments, the first sequence encoding a gRNA, the second sequence encoding a gRNA, and the third sequence encoding a gRNA are not identical.

In some embodiments, the AAV vector comprises a first sequence encoding a gRNA, a second sequence encoding a gRNA, a third sequence encoding a gRNA, and a fourth sequence encoding a gRNA, a first promoter drives expression of the first sequence encoding a gRNA, a second promoter drives expression of the second sequence encoding a gRNA, a third promoter drives expression of the third sequence encoding a gRNA, and a fourth promoter drives expression of the fourth sequence encoding a gRNA. In some embodiments, at least two of the first, second, third, and fourth promoters are the same. In some embodiments, each of the first, second, third, and fourth promoters are different. In some embodiments, each of the first, second, third and fourth promoters are selected from the H1 promoter, the U6 promoter, and the 7SK promoter. In some embodiments, the first sequence encoding a gRNA, the second sequence encoding a gRNA, the third sequence encoding a gRNA, and the fourth sequence encoding a gRNA are identical. In some embodiments, the first sequence encoding a gRNA, the second sequence encoding a gRNA, the third sequence encoding a gRNA, and the fourth sequence encoding a gRNA are not identical.

In some embodiments, the AAV vector comprises a first sequence encoding a gRNA, a second sequence encoding a gRNA, a third sequence encoding a gRNA, a fourth sequence encoding a gRNA, and a fifth sequence encoding a gRNA, a first promoter drives expression of the first sequence encoding a gRNA, a second promoter drives expression of the second sequence encoding a gRNA, a third promoter drives expression of the third sequence encoding a gRNA, a fourth promoter drives expression of the fourth sequence encoding a gRNA, and a fifth promoter drives expression of the fifth sequence encoding a gRNA. In some embodiments, at least two of the first, second, third, fourth, and fifth promoters are the same. In some embodiments, each of the first, second, third, fourth, and fifth promoters are different. In some embodiments, each of the first, second, third, and fourth promoters are different. In some embodiments, each of the first, second, third, fourth and fifth promoters are selected from the H1 promoter, the U6 promoter, and the 7SK promoter. In some embodiments, the first sequence encoding a gRNA, the second sequence encoding a gRNA, the third sequence encoding a gRNA, the fourth sequence encoding a gRNA, and the fifth sequence encoding a gRNA are identical. In some embodiments, the first sequence encoding a gRNA, the second sequence encoding a gRNA, the third sequence encoding a gRNA, the fourth sequence encoding a gRNA, and the fifth sequence encoding a gRNA are not identical.

Exemplary promoters of the disclosure include the U6 promoter having a sequence of CGAGTCCAACACCCGTGGGAATCCCATGGGCACCATGGCCCCTCGCTCCAAAAA TGCTTTCGCGTCGCGCAGACACTGCTCGGTAGTTTCGGGGATCAGCGTTTGAGTA AGAGCCCGCGTCTGAACCCTCCGCGCCGCCCCGGCCCCAGTGGAAAGACGCGCA GGCAAAACGCACCACGTGACGGAGCGTGACCGCGCGCCGAGCGCGCGCCAAGG TCGGGCAGGAAGAGGGCCTATTTCCCATGATTCCTTCATATTTGCATATACGATA CAAGGCTGTTAGAGAGATAATTAGAATTAATTTGACTGTAAACACAAAGATATT AGTACAAAATACGTGACGTAGAAAGTAATAATTTCTTGGGTAGTTTGCAGTTTTA AAATTATGTTTTAAAATGGACTATCATATGCTTACCGTAACTTGAAAGTATTTCG ATTTCTTGGCTTTATATATCTTGTGGAAAGGACGAAA (SEQ ID NO: 56), the H1 promoter having a sequence of GCTCGGCGCGCCCATATTTGCATGTCGCTATGTGTTCTGGGAAATCACCATAAAC GTGAAATGTCTTTGGATTTGGGAATCTTATAAGTTCTGTATGAGACCACGGTA (SEQ ID NO: 57), and the 7SK promoter having a sequence of TGACGGCGCGCCCTGCAGTATTTAGCATGCCCCACCCATCTGCAAGGCATTCTGG ATAGTGTCAAAACAGCCGGAAATCAAGTCCGTTTATCTCAAACTTTAGCATTTTG GGAATAAATGATATTTGCTATGCTGGTTAAATTAGATTTTAGTTAAATTTCCTGCT GAAGCTCTAGTACGATAAGTAACTTGACCTAAGTGTAAAGTTGAGATTTCCTTCA GGTTTATATAGCTTGTGCGCCGCCTGGGTA (SEQ ID NO: 58). In some embodiments, the construct comprising a first promoter, a first sequence encoding a gRNA, a second promoter, and a second sequence encoding a gRNA, a third promoter, and a third sequence encoding a gRNA further comprises at least two inverted terminal repeat (ITR) sequences. In some embodiments, the construct comprising a first promoter, a first sequence encoding a gRNA, a second promoter, and a second sequence encoding a gRNA, a third promoter, and a third sequence encoding a gRNA further comprises at least two ITR sequences isolated or derived from an AAV of serotype 2 (AAV2). In some embodiments, the construct comprising a first promoter, a first sequence encoding a gRNA, a second promoter, and a second sequence encoding a gRNA, a third promoter, and a third sequence encoding a gRNA further comprises at least two ITR sequences, wherein the first ITR sequence is isolated or derived from an AAV of serotype 4 (AAV4) and the second ITR sequence is isolated or derived from an AAV of serotype 2 (AAV2). Exemplary ITR sequences are CCTGCAGGCAGCTGCGCGCTCGCTCGCTCACTGAGGCCGCCCGGGCAAAGCCCG GGCGTCGGGCGACCTTTGGTCGCCCGGCCTCAGTGAGCGAGCGAGCGCGCAGAG AGGGAGTGGCCAACTCCATCACTAGGGGTTCCT (SEQ ID NO: 59), CCACTCCCTCTATGCGCGCTCGCTCACTCACTCGGCCCTGGAGACCAAAGGTCTC CAGACTGCCGGCCTCTGGCCGGCAGGGCCGAGTGAGTGAGCGAGCGCGCATAGA GGGAGTGGGTACCTCCATCATCTAGGTTTGCC (SEQ ID NO: 60), AGGAACCCCTAGTGATGGAGTTGGCCACTCCCTCTCTGCGCGCTCGCTCGCTCAC TGAGGCCGCCCGGGCAAAGCCCGGGCGTCGGGCGACCTTTGGTCGCCCGGCCTC AGTGAGCGAGCGAGCGCGCAGAGAGGGAGTGG (SEQ ID NO: 61), and AGGAACCCCTAGTGATGGAGTTGGCCACTCCCTCTCTGCGCGCTCGCTCGCTCAC TGAGGCCGGGCGACCAAAGGTCGCCCGACGCCCGGGCTTTGCCCGGGCGGCCTC AGTGAGCGAGCGAGCGCGCAGCTGCCTGCAGG (SEQ ID NO: 62). In some embodiments, the construct comprises or consists of, from 5′ to 3′, a first ITR, a first promoter, a first sequence encoding a gRNA, a second promoter, and a second sequence encoding a gRNA, a third promoter, a third sequence encoding a gRNA, and a second ITR. In some embodiments, the construct comprises or consists of, from 5′ to 3′, a first ITR, a U6 promoter, a first sequence encoding a gRNA, a H1 promoter, and a second sequence encoding a gRNA, a 7SK promoter, a third sequence encoding a gRNA, and a second ITR. In some embodiments, the construct comprising, from 5′ to 3′ a first ITR, a first promoter, a first sequence encoding a gRNA, a second promoter, and a second sequence encoding a gRNA, a third promoter, a third sequence encoding a gRNA, and a second ITR, further comprises a poly A sequence. In some embodiments, the polyA sequence comprises or consists of a minipolyA sequence. Exemplary minipolyA sequences of the disclosure comprise or consist of a nucleotide sequence of TAGCAATAAAGGATCGTTTATTTTCATTGGAAGCGTGTGTTGGTTTTTTGATCAGG CGCG (SEQ ID NO: 63). In some embodiments, the construct comprises or consists of, from 5′ to 3′, a first ITR, a first promoter, a first sequence encoding a gRNA, a second promoter, and a second sequence encoding s gRNA, a third promoter, a third sequence encoding a gRNA, a minipolyA sequence, and a second ITR. In some embodiments, the construct comprises or consists of, from 5′ to 3′ a first ITR, the U6 promoter, a first sequence encoding a gRNA, the H1 promoter, a second sequence encoding a gRNA, the 7SK promoter, a third sequence encoding a gRNA, a minipolyA sequence, and a second ITR. In some embodiments, the construct comprising, from 5′ to 3′ a first ITR, a first promoter, a first sequence encoding a gRNA, a second promoter, and a second sequence encoding a gRNA, a third promoter, a third sequence encoding a gRNA, a minipolyA sequence, and a second ITR further comprises transposable element inverted repeats. Exemplary transposable element inverted repeats of the disclosure comprise or consist of a nucleotide sequence of TGTGGGCGGACAAAATAGTTGGGAACTGGGAGGGGTGGAAATGGAGTTTTTAAG GATTATTTAGGGAAGAGTGACAAAATAGATGGGAACTGGGTGTAGCGTCGTAAG CTAATACGAAAATTAAAAATGACAAAATAGTTTGGAACTAGATTTCACTTATCTG GTT (SEQ ID NO: 64) and/or a nucleotide sequence of GAATATAGTCTTTACCATGCCCTTGGCCACGCCCCTCTTTAATACGACGGGCAAT TTGCACTTCAGAAAATGAAGAGTTTGCTTTAGCCATAACAAAAGTCCAGTATGCT TTTTCACAGCATAACTGGACTGATTTCAGTTTACAACTATTCTGTCTAGTTTAAGA CTTTATTGTCATAGTTTAGATCTATTTTGTTCAGTTTAAGACTTTATTGTCCGCCCA CA (SEQ ID NO: 65). In some embodiments, the construct comprises or consists of, from 5′ to 3′, a first transposable element inverted repeat, a first ITR, a first promoter, a first sequence encoding a gRNA, a second promoter, a second sequence encoding a gRNA, a third promoter, a third sequence encoding a gRNA, a minipolyA sequence, a second ITR, and a second transposable element inverted repeat. In some embodiments, the construct comprises or consists of, from 5′ to 3′, a first transposable element inverted repeat, a first ITR, the U6 promoter, a first sequence encoding a gRNA, the H1 promoter, a second sequence encoding a gRNA, the 7SK promoter, a third sequence encoding a gRNA, a minipolyA sequence, a second ITR, and a second transposable element inverted repeat. In some embodiments, the construct comprising a first transposable element inverted repeat, a first ITR, a first promoter, a first sequence encoding a gRNA, a second promoter, a second sequence encoding a gRNA, a third promoter, a third sequence encoding a gRNA, a minipolyA sequence, a second ITR, a second transposable element inverted repeat, further comprises a regulatory sequence. Exemplary regulatory sequences of the disclosure comprise or consist of a nucleotide sequence of CATGCAAGCTGTAGCCAACCACTAGAACTATAGCTAGAGTCCTGGGCGAACAAA CGATGCTCGCCTTCCAGAAAACCGAGGATGCGAACCACTTCATCCGGGGTCAGC ACCACCGGCAAGCGCCGCGACGGCCGAGGTCTTCCGATCTCCTGAAGCCAGGGC AGATCCGTGCACAGCACCTTGCCGTAGAAGAACAGCAAGGCCGCCAATGCCTGA CGATGCGTGGAGACCGAAACCTTGCGCTCGTTCGCCAGCCAGGACAGAAATGCC TCGACTTCGCTGCTGCCCAAGGTTGCCGGGTGACGCACACCGTGGAAACGGATG AAGGCACGAACCCAGTTGACATAAGCCTGTTCGGTTCGTAAACTGTAATGCAAG TAGCGTATGCGCTCACGCAACTGGTCCAGAACCTTGACCGAACGCAGCGGTGGT AACGGCGCAGTGGCGGTTTTCATGGCTTGTTATGACTGTTTTTTTGTACAGTCTAT GCCTCGGGCATCCAAGCAGCAAGCGCGTTACGCCGTGGGTCGATGTTTGATGTTA TGGAGCAGCAACGATGTTACGCAGCAGCAACGATGTTACGCAGCAGGGCAGTCG CCCTAAAACAAAGTTAGGTGGCTCAAGTATGGGCATCATTCGCACATGTAGGCTC GGCCCTGACCAAGTCAAATCCATGCGGGCTGCTCTTGATCTTTTCGGTCGTGAGT TCGGAGACGTAGCCACCTACTCCCAACATCAGCCGGACTCCGATTACCTCGGGA ACTTGCTCCGTAGTAAGACATTCATCGCGCTTGCTGCCTTCGACCAAGAAGCGGT TGTTGGCGCTCTCGCGGCTTACGTTCTGCCCAAGTTTGAGCAGCCGCGTAGTGAG ATCTATATCTATGATCTCGCAGTCTCCGGCGAGCACCGGAGGCAGGGCATTGCCA CCGCGCTCATCAATCTCCTCAAGCATGAGGCCAACGCGCTTGGTGCTTATGTGAT CTACGTGCAAGCAGATTACGGTGACGATCCCGCAGTGGCTCTCTATACAAAGTTG GGCATACGGGAAGAAGTGATGCACTTTGATATCGACCCAAGTACCGCCACCTAA CAATTCGTTCAAGCCGAGATCGGCTTCCCGGCCGCGGAGTTGTTCGGTAAATTGT CACAACGCCG (SEQ ID NO: 66). In some embodiments, the construct comprises or consists of, from 5′ to 3′ a first transposable element inverted repeat, a first ITR, a first promoter, a first sequence encoding a gRNA, a second promoter, a second sequence encoding a gRNA, a third promoter, a third sequence encoding a gRNA, a minipolyA sequence, a second ITR, a regulatory sequence, and a second transposable element inverted repeat. In some embodiments, the construct comprises or consists of, from 5′ to 3′ a first transposable element inverted repeat, a first ITR, the U6 promoter, a first sequence encoding a gRNA, the H1 promoter, a second sequence encoding a gRNA, the 7SK promoter, a third sequence encoding a gRNA, a minipolyA sequence, a second ITR, a regulatory sequence, and a second transposable element inverted repeat. In some embodiments, the construct comprising a first ITR, a first promoter, a first sequence encoding a gRNA, a second promoter, and a second sequence encoding a gRNA, a third promoter, a third sequence encoding a gRNA, a minipolyA sequence, and a second ITR, further comprises a stuffer sequence. In some embodiments, the construct comprises or consists of, from 5′ to 3′ a first ITR, the U6 promoter, a first sequence encoding a gRNA, the H1 promoter, a second sequence encoding a gRNA, the 7SK promoter, a third sequence encoding a gRNA, a stuffer sequence, a minipolyA sequence, and a second ITR. In some embodiments, the construct comprises or consists of, from 5′ to 3′ a first transposable element inverted repeat, a first ITR, a first promoter, a first sequence encoding a gRNA, a second promoter, a second sequence encoding a gRNA, a third promoter, a third sequence encoding a gRNA, a stuffer sequence, a minipolyA sequence, a second ITR, a regulatory sequence, and a second transposable element inverted repeat. In some embodiments, the construct comprises or consists of, from 5′ to 3′ a first transposable element inverted repeat, a first ITR, the U6 promoter, a first sequence encoding a gRNA, the H1 promoter, a second sequence encoding a gRNA, the 7SK promoter, a third sequence encoding a gRNA, a stuffer sequence, a minipolyA sequence, a second ITR, a regulatory sequence, and a second transposable element inverted repeat. In some embodiments, the construct may further comprise one or more spacer sequences. Exemplary spacer sequences of the disclosure have length from 1-1500 nucleotides, inclusive of all ranges therebetween. In some embodiments, the spacer sequences may be located at a position that is 5′ to or 3′ to an ITR, a promoter, a sequence encoding a gRNA, a polyA sequence, a transposable element inverted repeat, a stuffer sequence, and/or a regulator element.

### IV. EXEMPLARY THERAPEUTIC NUCLEIC ACIDS AND VECTORS

In some embodiments, a nucleic acid comprises a sequence encoding a first DMD guide RNA targeting a first genomic target sequence, a sequence encoding a second DMD guide RNA targeting a second genomic target sequence, a sequence encoding a first promoter wherein the first promoter drives expression of the sequence encoding the first DMD guide RNA, and a sequence encoding a second promoter wherein the first promoter drives expression of the sequence encoding the second DMD guide RNA, wherein the first genomic target sequence and the second genomic target sequence each comprise a dystrophin splice acceptor site.

In some embodiments, the sequence encoding the first promoter and the sequence encoding the second promoter are identical. In some embodiments, the sequence encoding the first promoter and the sequence encoding the second promoter not identical. In some embodiments, the sequence encoding the first promoter and the sequence encoding the second promoter share at least 50%, 60%, 70%, 80%, 90%, or 95% sequence identity.

In some embodiments, the first genomic target sequence and the second genomic target sequence are identical. In some embodiments, the first genomic target sequence and the second genomic target sequence are not identical. In some embodiments, the first genomic target sequence and the second genomic target sequence share at least 50%, 60%, 70%, 80%, 90%, or 95% sequence identity. In some embodiments, the first genomic target sequence and the second genomic target sequence are complementary.

In some embodiments, the nucleic acid further comprises a sequence encoding a third DMD guide RNA targeting a third genomic target sequence, and a sequence encoding a third promoter wherein the third promoter drives expression of the sequence encoding the third DMD guide RNA, and wherein the third genomic target sequence comprises a dystrophin splice acceptor site. In some embodiments, at least two of the sequences encoding the first promoter, the sequence encoding the second promoter, and the sequence encoding the third promoter are identical. In some embodiments, at least two of the sequence encoding the first promoter, the sequence encoding the second promoter, and the sequence encoding the third promoter are not identical. In some embodiments, at least two of the sequences encoding the first promoter, the sequence encoding the second promoter, and the sequence encoding the third promoter share at least 50%, 60%, 70%, 80%, 90%, or 95% sequence identity. In some embodiments, at least two of the first genomic target sequence, the second genomic target sequence, and the third genomic target sequence are identical. In some embodiments, at least two of the first genomic target sequence, the second genomic target sequence, and the third genomic target sequence are not identical. In some embodiments, at least two of the first genomic target sequence, the second genomic target sequence, and the third genomic target sequence share at least 50%, 60%, 70%, 80%, 90%, or 95% sequence identity. In some embodiments, at least two of the first genomic target sequence, the second genomic target sequence, and the third genomic target sequence are complementary.

In some embodiments, the nucleic acid further comprises a sequence encoding a fourth DMD guide RNA targeting a fourth genomic target sequence, and a sequence encoding a fourth promoter, wherein the fourth promoter drives expression of the fourth sequence encoding a DMD guide RNA, wherein the fourth genomic target sequence comprises a dystrophin splice acceptor site. In some embodiments, at least two of the sequence encoding the first promoter, the sequence encoding the second promoter, the sequence encoding the third promoter, and the sequence encoding the fourth promoter are identical. In some embodiments, at least two of the sequence encoding the first promoter, the sequence encoding the second promoter, the sequence encoding the third promoter, and the sequence encoding the fourth promoter are not identical. In some embodiments, at least two of the sequence encoding the first promoter, the sequence encoding the second promoter, the sequence encoding the third promoter, and the sequence encoding the fourth promoter share at least 50%, 60%, 70%, 80%, 90%, or 95% sequence identity. In some embodiments, at least two of the first genomic target sequence, the second genomic target sequence, the third genomic target sequence, and the fourth genomic target sequence are identical. In some embodiments, at least two of the first genomic target sequence, the second genomic target sequence, the third genomic target sequence, and the fourth genomic target sequence are not identical. In some embodiments, at least two of the first genomic target sequence, the second genomic target sequence, the third genomic target sequence, and the fourth genomic target sequence share at least 50%, 60%, 70%, 80%, 90%, or 95% sequence identity. In some embodiments, at least two of the first genomic target sequence, the second genomic target sequence, the third genomic target sequence, and the fourth genomic target sequence are complementary.

In some embodiments, the nucleic acid further comprises a sequence encoding a fifth DMD guide RNA targeting a fifth genomic target sequence, and a sequence encoding a fifth promoter, wherein the fifth promoter drives expression of the sequence encoding the fifth DMD guide RNA, wherein the fifth genomic target sequence comprises a dystrophin splice acceptor site. In some embodiments, at least two of the sequence encoding the first promoter, the sequence encoding the second promoter, the sequence encoding the third promoter, the sequence encoding the fourth promoter, and the sequence encoding the fifth promoter are identical. In some embodiments, at least two of the sequence encoding the first promoter, the sequence encoding the second promoter, the sequence encoding the third promoter, the sequence encoding the fourth promoter, and the sequence encoding the fifth promoter are not identical. In some embodiments, at least two of the sequence encoding the first promoter, the sequence encoding the second promoter, the sequence encoding the third promoter, the sequence encoding the fourth promoter, and the sequence encoding the fifth promoter share at least 50%, 60%, 70%, 80%, 90%, or 95% sequence identity. In some embodiments, at least two of the first genomic target sequence, the second genomic target sequence, the third genomic target sequence, the fourth genomic target sequence, and the fifth genomic target sequence are identical. In some embodiments, at least two of the first genomic target sequence, the second genomic target sequence, the third genomic target sequence, the fourth genomic target sequence, and the fifth genomic target sequence are not identical. In some embodiments, at least two of the first genomic target sequence, the second genomic target sequence, the third genomic target sequence, the fourth genomic target sequence, and the fifth genomic target sequence share at least 50%, 60%, 70%, 80%, 90%, or 95% sequence identity. In some embodiments, at least two of the first genomic target sequence, the second genomic target sequence, the third genomic target sequence, the fourth genomic target sequence, and the fifth genomic target sequence are complementary.

In some embodiments, the nucleic acid further comprises at least one sequence encoding an additional DMD guide RNA targeting a genomic target sequence, and at least one additional promoter, wherein the additional promoter drives expression of the sequence encoding the additional DMD guide RNA, wherein the additional genomic target sequence comprises a dystrophin splice acceptor site. In some embodiments, the dystrophin splice acceptor site comprises the 5′ splice acceptor site of exon 51. In some embodiments, the sequence encoding the first promoter or the sequence encoding the second promoter comprises a sequence encoding a constitutive promoter. In some embodiments, the first promoter or the second promoter comprises a constitutive promoter. In some embodiments, at least one of the sequence encoding the first promoter, the sequence encoding the second promoter, the sequence encoding the third promoter, the sequence encoding the fourth promoter, and the sequence encoding the fifth promoter comprises a sequence encoding a constitutive promoter. In some embodiments, at least one of the first promoter, the second promoter, the third promoter, the fourth promoter, and the fifth promoter comprises a constitutive promoter. In some embodiments, the sequence encoding the first promoter or the sequence encoding the second promoter comprises a sequence encoding an inducible promoter. In some embodiments, at least one of the first promoter, the second promoter, the third promoter, the fourth promoter, and the fifth promoter comprises an inducible promoter. In some embodiments, the sequence encoding the first promoter or the sequence encoding the second promoter comprises a sequence encoding a cell-type specific promoter. In some embodiments, at least one of the sequence encoding the first promoter, the sequence encoding the second promoter, the sequence encoding the third promoter, the sequence encoding the fourth promoter, and the sequence encoding the fifth promoter comprises a cell-type specific promoter. In some embodiments, the cell type specific promoter comprises a muscle-specific promoter. In some embodiments, the sequence encoding the first promoter or the sequence encoding the second promoter comprises a sequence encoding a U6 promoter, an H1 promoter, or a 7SK promoter. In some embodiments, at least one of the sequence encoding the first promoter, the sequence encoding the second promoter, the sequence encoding the third promoter, the sequence encoding the fourth promoter, and the sequence encoding the fifth promoter comprises a U6 promoter, an H1 promoter, or a 7SK promoter. In some embodiments, at least one of the sequence encoding the first promoter, the sequence encoding the second promoter, the sequence encoding the third promoter, the sequence encoding the fourth promoter, and the sequence encoding the fifth promoter comprises a U6 promoter. In some embodiments, at least one of the sequence encoding the first promoter, the sequence encoding the second promoter, the sequence encoding the third promoter, the sequence encoding the fourth promoter, and the sequence encoding the fifth promoter comprises an H1 promoter. In some embodiments, at least one of the sequence encoding the first promoter, the sequence encoding the second promoter, the sequence encoding the third promoter, the sequence encoding the fourth promoter, and the sequence encoding the fifth promoter comprises a 7SK promoter.

In some embodiments, the sequence encoding the first DMD guide RNA, the sequence encoding the second DMD guide RNA, and sequence encoding the third DMD guide RNA are identical, and the 5′ splice acceptor site comprises a 5′ splice acceptor site of exon 51. In some embodiments, the sequence encoding the first promoter comprises a sequence encoding a U6 promoter, the sequence encoding the second promoter comprises a sequence encoding an H1 promoter, and the sequence encoding the third promoter comprises a 7SK promoter. In some embodiments, the nucleic acid comprises a DNA sequence. In some embodiments, the nucleic acid comprises an RNA sequence.

In some embodiments, the nucleic acid further comprises one or more sequences encoding an inverted terminal repeat (ITR). In some embodiments, the nucleic acid further comprises a sequence encoding a 5′ inverted terminal repeat (ITR) and a sequence encoding a 3′ ITR. In some embodiments, the sequence encoding the 5′ inverted terminal repeat (ITR) or the sequence encoding a 3′ ITR comprises a sequence isolated or derived from an adeno-associated virus (AAV). In some embodiments, the sequence encoding the 5′ inverted terminal repeat (ITR) or the sequence encoding a 3′ ITR comprises a sequence isolated or derived from an adeno-associated virus (AAV) of serotype 2 (AAV2). In some embodiments, the sequence encoding the 5′ inverted terminal repeat (ITR) and the sequence encoding a 3′ ITR comprises a sequence isolated or derived from an AAV2. In some embodiments, the sequence encoding the 5′ inverted terminal repeat (ITR) or the sequence encoding a 3′ ITR comprises a sequence isolated or derived from an adeno-associated virus (AAV) of serotype 4 (AAV4). In some embodiments, the sequence encoding the 5′ inverted terminal repeat (ITR) and the sequence encoding a 3′ ITR comprises a sequence isolated or derived from an AAV4. In some embodiments, the sequence encoding the 5′ inverted terminal repeat (ITR) or the sequence encoding a 3′ ITR comprises or consists of 145 nucleotides. In some embodiments, the sequence encoding the 5′ inverted terminal repeat (ITR) or the sequence encoding a 3′ ITR comprises or consists of 115 nucleotides. In some embodiments, the sequence encoding the 5′ inverted terminal repeat (ITR) or the sequence encoding a 3′ ITR comprises or consists of 141 nucleotides. In some embodiments, the nucleic acid further comprises a polyadenosine (poly A) sequence. In some embodiments, the poly A sequence is a mini poly A sequence.

Also provided is a vector comprising a nucleic acid comprising a sequence encoding a first DMD guide RNA targeting a first genomic target sequence, a sequence encoding a second DMD guide RNA targeting a second genomic target sequence, a sequence encoding a first promoter wherein the first promoter drives expression of the sequence encoding the first DMD guide RNA, and a sequence encoding a second promoter wherein the first promoter drives expression of the sequence encoding the second DMD guide RNA, wherein the first genomic target sequence and the second genomic target sequence each comprise a dystrophin splice acceptor site. In some embodiments, the vector further comprises a sequence encoding an inverted terminal repeat of a transposable element. In some embodiments, the transposable element is a transposon. In some embodiments, the transposon is a Tn7 transposon. In some embodiments, the vector is a non-viral vector. In some embodiments, the non-viral vector is a plasmid. In some embodiments, the vector is a viral vector. In some embodiments, the viral vector is an adeno-associated viral (AAV) vector. In some embodiments, the AAV vector is replication-defective or conditionally replication defective. In some embodiments, the AAV vector is a recombinant AAV vector. In some embodiments, the AAV vector comprises a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11), AAVrh74, AAVrh10 or any combination thereof. In some embodiments, the AAV vector comprises a sequence isolated or derived from an AAV vector of serotype 9 (AAV9). In some embodiments, the AAV vector comprises a sequence isolated or derived from an AAV vector of serotype 2 (AAV2). In some embodiments, the AAV vector comprises a sequence isolated or derived from an AAV2 and a sequence isolated or derived from an AAV9. In some embodiments, the AAV vector comprises a sequence isolated or derived from an AAV4 and a sequence isolated or derived from an AAV9. In some embodiments, the vector is optimized for expression in mammalian cells. In some embodiments, the vector is optimized for expression in human cells.

Also provided is a nucleic acid comprising a sequence encoding a promoter and a sequence encoding a Cas9 or a nuclease domain thereof, wherein the sequence encoding the promoter comprises a sequence encoding a muscle-specific promoter. In some embodiments, the sequence encoding the muscle-specific promoter comprises a sequence encoding a CK8 promoter. In some embodiments, the sequence encoding the muscle-specific promoter comprises a sequence encoding a CK8e promoter. In some embodiments, the sequence encoding the Cas9 or the nuclease domain thereof is isolated or derived from a sequence encoding an S. pyogenes Cas9 or a nuclease domain thereof. In some embodiments, the sequence encoding the Cas9 or the nuclease domain thereof is isolated or derived from a sequence encoding S. aureus Cas9 or a nuclease domain thereof. In some embodiments, the sequence encoding the Cas9 or the nuclease domain thereof is codon optimized for expression in a mammal. In some embodiments, the sequence encoding the Cas9 or the nuclease domain thereof is codon optimized for expression in a human.

In some embodiments, the nucleic acid comprising a sequence encoding a promoter and a sequence encoding a Cas9 or a nuclease domain thereof further comprises a polyA sequence. In some embodiments, the polyA sequence is a mini polyA sequence. In some embodiments, the nucleic acid further comprises one or more sequences encoding an inverted terminal repeat (ITR). In some embodiments, the nucleic acid further comprises a sequence encoding a 5′ inverted terminal repeat (ITR) and a sequence encoding a 3′ ITR. In some embodiments, the sequence encoding the 5′ inverted terminal repeat (ITR) or the sequence encoding a 3′ ITR comprises a sequence isolated or derived from an adeno-associated virus (AAV). In some embodiments, the sequence encoding the 5′ inverted terminal repeat (ITR) or the sequence encoding a 3′ ITR comprises a sequence isolated or derived from an adeno-associated virus (AAV) of serotype 2 (AAV2). In some embodiments, the sequence encoding the 5′ inverted terminal repeat (ITR) and the sequence encoding a 3′ ITR comprises a sequence isolated or derived from an AAV2. In some embodiments, the sequence encoding the 5′ inverted terminal repeat (ITR) or the sequence encoding a 3′ ITR comprises a sequence isolated or derived from an adeno-associated virus (AAV) of serotype 4 (AAV4). In some embodiments, the sequence encoding the 5′ inverted terminal repeat (ITR) and the sequence encoding a 3′ ITR comprises a sequence isolated or derived from an AAV4. In some embodiments, the sequence encoding the 5′ inverted terminal repeat (ITR) or the sequence encoding a 3′ ITR comprises or consists of 145 nucleotides, 115 nucleotides, or 141 nucleotides. In some embodiments, the nucleic acid further comprises a nuclear localization signal. In some embodiments, the nucleic acid is optimized for expression in mammalian cells. In some embodiments, the nucleic acid is optimized for expression in human cells.

Also provided is a vector comprising a nucleic acid comprising a sequence encoding a promoter and a sequence encoding a Cas9 or a nuclease domain thereof, wherein the sequence encoding the promoter comprises a sequence encoding a muscle-specific promoter such as the CK8 or CK8e promoter. In some embodiments, the vector further comprises a sequence encoding an inverted terminal repeat (ITR) of a transposable element. In some embodiments, the transposable element is a transposon. In some embodiments, the transposon is a Tn7 transposon. In some embodiments, the vector further comprises a sequence encoding a 5′ ITR of a T7 transposon and a sequence encoding a 3′ ITR of a T7 transposon. In some embodiments, the vector is a non-viral vector. In some embodiments, the non-viral vector is a plasmid. In some embodiments, the vector is a viral vector. In some embodiments, the viral vector is an adeno-associated viral (AAV) vector. In some embodiments, the AAV vector is replication-defective or conditionally replication defective. In some embodiments, the AAV vector is a recombinant AAV vector. In some embodiments, the AAV vector comprises a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11), AAVrh74, AAVrh10 or any combination thereof. In some embodiments, the AAV vector comprises a sequence isolated or derived from an AAV vector of serotype 9 (AAV9). In some embodiments, the AAV vector comprises a sequence isolated or derived from an AAV vector of serotype 2 (AAV2). In some embodiments, the AAV vector comprises a sequence isolated or derived from an AAV2 and a sequence isolated or derived from an AAV9. In some embodiments, the AAV vector comprises a sequence isolated or derived from an AAV vector of serotype 4 (AAV4). In some embodiments, the AAV vector comprises a sequence isolated or derived from an AAV4 and a sequence isolated or derived from an AAV9. In some embodiments, wherein the vector is optimized for expression in mammalian cells. In some embodiments, the vector is optimized for expression in human cells.

### V. PHARMACEUTICAL COMPOSITIONS AND DELIVERY METHODS

Also provided herein are compositions comprising one or more vectors and/or nucleic acids of the disclosure. In some embodiments, the composition further comprises a pharmaceutically acceptable carrier.

For clinical applications, pharmaceutical compositions are prepared in a form appropriate for the intended application. Generally, this entails preparing compositions that are essentially free of pyrogens, as well as other impurities that could be harmful to humans or animals.

Appropriate salts and buffers are used to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Any conventional media or agent that is not incompatible with the active ingredients of the present disclosure, its use in therapeutic compositions may be used. Supplementary active ingredients also can be incorporated into the compositions, provided they do not inactivate the vectors or cells of the compositions.

In some embodiments, the active compositions of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

In some embodiments, the compositions of the present disclosure are formulated in a neutral or salt form. Pharmaceutically acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine) and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, a first vector and a second vector are administered to a patient. In some embodiments, the first vector comprises a nucleic acid comprising a first sequence encoding a first DMD guide RNA targeting a first genomic target sequence; a sequence encoding a second DMD guide RNA targeting a second genomic target sequence; a sequence encoding a first promoter, wherein the first promoter drives expression of the sequence encoding the first DMD guide RNA; and a sequence encoding a second promoter, wherein the second promoter drives expression of the sequence encoding the second DMD guide RNA. In some embodiments, the second vector comprise a nucleic acid comprising a sequence encoding a Cas9 or a nuclease domain thereof; a sequence encoding a muscle-specific promoter, wherein the muscle-specific promoter drives expression of the sequence encoding a Cas9 or a nuclease domain thereof.

### VI. THERAPEUTICALLY-EFFECTIVE RATIOS

In some embodiments, a first vector and a second vector are administered to a patient in a therapeutically effective ratio. As used herein, the term “ratio” may refer to a ratio of the amount of vector in a composition (concentration), amount delivered to a patient (dosage), amount available to a therapeutic site (bioavailability), amount expressed by a target cell (copy number), amount of modifications made (efficacy), amount of DNA, or number of coding sequences (e.g., sequences encoding a gRNA or a Cas9).

In some embodiments, the ratio of the first vector and the second vector is between 1:1 and 1:30. In other embodiments, the ratio of the first vector and the second vector is between 30:1 and 1:1. In some embodiments, the ratio of the amount of the first vector and amount of the second vector is between 1:1 and 1:30. In other embodiments, the ratio of the amount of the first vector and amount of the second vector is between 30:1 and 1:1. In some embodiments, the first vector is an AAV-Cas9 vector of the disclosure and the second vector is an AAV-sgRNA vector of the disclosure.

In some embodiments, the ratio of the first vector to the second vector is greater than 10:1. For example, the ratio of the first vector to the second vector may be about 11:1, about 12:1, about 13:1, about 14:1, about 15:1, about 16:1, about 17:1, about 18:1, about 19:1, about 20:1, about 25:1, about 30:1, about 35:1, about 40:1, about 50:1, about 75:1, or about 100:1. In some embodiments, the ratio of an AAV-sgRNA vector to an AAV-Cas9 vector is greater than 10:1; for example, the ratio may be about 11:1, about 12:1, about 13:1, about 14:1, about 15:1, about 16:1, about 17:1, about 18:1, about 19:1, about 20:1, about 25:1, about 30:1, about 35:1, about 40:1, about 50:1, about 75:1, or about 100:1.

In some embodiments, between 4×10¹² viral genomes (vg)/kilogram (kg) and 3×10¹³ vg/kg, inclusive of the endpoints, of the first and/or the second vector are administered to the patient. In some embodiments, between 4×10¹² viral genomes (vg)/kilogram (kg) and 3×10¹³ vg/kg, inclusive of the endpoints, of the first and/or the second vector are administered to the patient. In some embodiments, at least 5×10¹² viral genomes (vg)/kilogram (kg), 6×10¹² viral genomes (vg)/kilogram (kg), 1×10¹³ viral genomes (vg)/kilogram (kg), 2×10¹³ viral genomes (vg)/kilogram (kg), 3×10¹³ viral genomes (vg)/kilogram (kg), 5×10¹³ viral genomes (vg)/kilogram (kg), 1×10¹⁴ viral genomes (vg)/kilogram (kg), 2×10¹⁴ viral genomes (vg)/kilogram (kg), 3×10¹⁴ viral genomes (vg)/kilogram (kg), or 4×10¹⁴ viral genomes (vg)/kilogram (kg) of the first and/or the second vector are administered to the patient.

In some embodiments, the Cas9 and gRNAs described herein may be delivered to the patient using adoptive cell transfer (ACT). In adoptive cell transfer, one or more expression constructs are provided ex vivo to cells which have originated from the patient (autologous) or from one or more individual(s) other than the patient (allogeneic). The cells are subsequently introduced or reintroduced into the patient. Thus, in some embodiments, one or more nucleic acids encoding Cas9 and a guide RNA that targets a dystrophin splice site are provided to a cell ex vivo before the cell is introduced or reintroduced to a patient.

In some embodiments, a composition comprises (i) a first nucleic acid sequence comprising a sequence encoding a first DMD guide RNA targeting a first genomic target sequence, a sequence encoding a second DMD guide RNA targeting a second genomic target sequence, a sequence encoding a first promoter wherein the first promoter drives expression of the sequence encoding the first DMD guide RNA, and a sequence encoding a second promoter wherein the first promoter drives expression of the sequence encoding the second DMD guide RNA, wherein the first genomic target sequence and the second genomic target sequence each comprise a dystrophin splice acceptor and (ii) a second nucleic acid sequence comprising a sequence encoding a promoter and a sequence encoding a Cas9 or a nuclease domain thereof, wherein the sequence encoding the promoter comprises a sequence encoding a muscle-specific promoter such as the CK8 or CK8e promoter. In some embodiments, the composition further comprises a pharmaceutically acceptable carrier.

In some embodiments, a composition comprises (i) a first vector comprising a nucleic acid sequence comprising a sequence encoding a first DMD guide RNA targeting a first genomic target sequence, a sequence encoding a second DMD guide RNA targeting a second genomic target sequence, a sequence encoding a first promoter wherein the first promoter drives expression of the sequence encoding the first DMD guide RNA, and a sequence encoding a second promoter wherein the first promoter drives expression of the sequence encoding the second DMD guide RNA, wherein the first genomic target sequence and the second genomic target sequence each comprise a dystrophin splice acceptor and (ii) a second vector comprising a nucleic acid sequence comprising a sequence encoding a promoter and a sequence encoding a Cas9 or a nuclease domain thereof, wherein the sequence encoding the promoter comprises a sequence encoding a muscle-specific promoter such as the CK8 or CK8e promoter. In some embodiments, at least one of the first vector and the second vectors are AAVs. In some embodiments, the composition further comprises a pharmaceutically acceptable carrier.

### VII. CELLS AND CELL COMPOSITIONS

Also provided is a cell comprising one or more nucleic acids of the disclosure. In some embodiments, the cell is a human cell. In some embodiments, the cell is a muscle cell or satellite cell. In some embodiments, the cell is an induced pluripotent stem (iPS) cell. Also provided is a composition comprising a cell comprising one or more nucleic acids of the disclosure. In some embodiments, the composition further comprises a pharmaceutically acceptable carrier.

Also provided is a cell comprising a composition comprising one or more vectors of the disclosure. In some embodiments, the cell is a human cell. In some embodiments, the cell is a muscle cell or satellite cell. In some embodiments, the cell is an induced pluripotent stem (iPS) cell.

### VIII. THERAPEUTIC METHODS AND USES

Also provided is a method for correcting a dystrophin defect, the method comprising contacting a cell with one or more compositions of the disclosure under conditions suitable for expression of the first DMD guide RNA, the second DMD guide RNA and the Cas9 protein or a nuclease domain thereof, wherein at least one of first DMD guide RNA or the second DMD guide RNA forms a complex with the Cas9 protein or the nuclease domain thereof to form at least one DMD guide RNA-Cas9 complex, wherein the at least one DMD guide RNA-Cas9 complex disrupts a dystrophin splice site and induces selective skipping of a DMD exon and/or reframing. In some embodiments, the at least one DMD guide RNA-Cas9 complex disrupts a dystrophin splice site and induces a reframing of a dystrophin reading frame. In some embodiments, the at least one DMD guide RNA-Cas9 complex disrupts a dystrophin splice site and produces an insertion which restores the dystrophin protein reading frame. In some embodiments, the insertion comprises an insertion of a single adenosine.

Also provided is a method for inducing selective skipping and/or reframing of a DMD exon, the method comprising contacting a cell with one or more compositions of the disclosure under conditions suitable for expression of the first DMD guide RNA, the second DMD guide RNA and the Cas9 protein or a nuclease domain thereof, wherein at least one of first DMD guide RNA or the second DMD guide RNA forms a complex with the Cas9 protein or the nuclease domain thereof to form at least one DMD guide RNA-Cas9 complex, wherein the at least one DMD guide RNA-Cas9 complex disrupts a dystrophin splice site and induces selective skipping and/or reframing of a DMD exon.

Also provided is a method for inducing a reframing event in the dystrophin reading frame, the method comprising contacting a cell with one or more compositions of the disclosure under conditions suitable for expression of the first DMD guide RNA, the second DMD guide RNA and the Cas9 protein or a nuclease domain thereof, wherein at least one of first DMD guide RNA or the second DMD guide RNA forms a complex with the Cas9 protein or the nuclease domain thereof to form at least one DMD guide RNA-Cas9 complex, wherein the at least one DMD guide RNA-Cas9 complex disrupts a dystrophin splice site and induces selective skipping and/or reframing of a DMD exon. In some embodiments, the at least one DMD guide RNA-Cas9 complex disrupts a dystrophin splice site and induces selective skipping and/or reframing of exon 51 of a human DMD gene.

Also provided is a method of treating muscular dystrophy in a subject in need thereof comprising administering to the subject a therapeutically effective amount of one or more compositions of the disclosure. In some embodiments, the composition is administered locally. In some embodiments, the composition is administered directly to a muscle tissue. In some embodiments, the composition is administered by an intramuscular infusion or injection. In some embodiments, the muscle tissue comprises a tibialis anterior tissue, a quadriceps tissue, a soleus tissue, a diaphragm tissue, or a heart tissue. In some embodiments, the composition is administered by an intra-cardiac injection. In some embodiments, the composition is administered systemically. In some embodiments, the composition is administered by an intravenous infusion or injection. In some embodiments, following administration of the composition, the subject exhibits normal dystrophin-positive myofibers, and mosaic dystrophin-positive myofibers containing centralized nuclei, or a combination thereof. In some embodiments, following administration of the composition, the subject exhibits an emergence or an increase in a level of abundance of normal dystrophin-positive myofibers when compared to an absence or a level of abundance of normal dystrophin-positive myofibers prior to administration of the composition. In some embodiments, following administration of the composition, the subject exhibits an emergence or an increase in a level of abundance of mosaic dystrophin-positive myofibers containing centralized nuclei when compared to an absence or an level of abundance of mosaic dystrophin-positive myofibers containing centralized nuclei prior to administration of the composition. In some embodiments, following administration of the composition, the subject exhibits a decreased serum CK level when compared to a serum CK level prior to administration of the composition. In some embodiment, following administration of the composition, the subject exhibits improved grip strength when compared to a grip strength prior to administration of the composition. In some embodiments, the subject is a neonate, an infant, a child, a young adult, or an adult. In some embodiments, the subject has muscular dystrophy. In some embodiments, the subject is a genetic carrier for muscular dystrophy. In some embodiments, the subject is male. In some embodiments, the subject is female. In some embodiments, the subject appears to be asymptomatic and a genetic diagnosis reveals a mutation in one or both copies of a DMD gene that impairs function of the DMD gene product. In some embodiments, the subject presents an early sign or symptom of muscular dystrophy. In some embodiments, the early sign or symptom of muscular dystrophy comprises loss of muscle mass or proximal muscle weakness. In some embodiments, the loss of muscle mass or proximal muscle weakness occurs in one or both leg(s) and/or a pelvis, followed by one or more upper body muscle(s). In some embodiments, the early sign or symptom of muscular dystrophy further comprises pseudohypertrophy, low endurance, difficulty standing, difficulty walking, difficulty ascending a staircase or a combination thereof. In some embodiments, the subject presents a progressive sign or symptom of muscular dystrophy. In some embodiments, the progressive sign or symptom of muscular dystrophy comprises muscle tissue wasting, replacement of muscle tissue with fat, or replacement of muscle tissue with fibrotic tissue. In some embodiments, the subject presents a later sign or symptom of muscular dystrophy. In some embodiments, the later sign or symptom of muscular dystrophy comprises abnormal bone development, curvature of the spine, loss of movement, and paralysis. In some embodiments, the subject presents a neurological sign or symptom of muscular dystrophy. In some embodiments, the neurological sign or symptom of muscular dystrophy comprises intellectual impairment and paralysis. In some embodiments, administration of the composition occurs prior to the subject presenting one or more progressive, later or neurological signs or symptoms of muscular dystrophy. In some embodiments, the subject is less than 10 years old, less than 5 years old, or less than 2 years old.

Also provided is the use of a therapeutically effective amount of one or more compositions of the disclosure for treating muscular dystrophy in a subject in need thereof.

Tables 6-25 provide exemplary primer and genomic targeting sequences for use in connection with the compositions and methods disclosed herein.

### IX. DELIVERY OF VECTORS

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation. In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Improved methods for culturing 293 cells and propagating adenovirus are known in the art. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/1) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenoviruses of the disclosure are replication defective, or at least conditionally replication defective. The adenovirus may be of any of the 42 different known serotypes or subgroups A-F. Adenovirus type 5 of subgroup C is the preferred starting material in order to obtain the conditional replication-defective adenovirus vector for use in the present disclosure.

As stated above, the typical vector according to the present disclosure is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors, or in the E4 region where a helper cell line or helper virus complements the E4 defect.

Adenovirus is easy to grow and manipulate and exhibits broad host range in vitro and in vivo. This group of viruses can be obtained in high titers, e.g., 109-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and therapeutic potential as in vivo gene transfer vectors.

Adenovirus vectors have been used in eukaryotic gene expression and vaccine development. Animal studies suggested that recombinant adenovirus could be used for gene therapy. Studies in administering recombinant adenovirus to different tissues include trachea instillation, muscle injection, peripheral intravenous injections and stereotactic inoculation into the brain.

The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses may be used, in which biotinylated antibodies against a retroviral envelope protein and against a specific cell receptor are used. The antibodies are coupled via the biotin components by using streptavidin. Using antibodies against major histocompatibility complex class I and class II antigens, it has been demonstrated the infection of a variety of human cells that bore those surface antigens with an ecotropic virus in vitro (Roux et al., 1989).

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes. Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (see, for example, Markowitz et al., 1988; Hersdorffer et al., 1990).

Other viral vectors may be employed as expression constructs in the present disclosure. Vectors derived from viruses such as vaccinia virus adeno-associated virus (AAV) and herpesviruses may be employed. They offer several attractive features for various mammalian cells.

In embodiments, the AAV vector is replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

In some embodiments, a single viral vector is used to deliver a nucleic acid encoding a Cas9 and at least one gRNA to a cell. In some embodiments, Cas9 is provided to a cell using a first viral vector and at least one gRNA is provided to the cell using a second viral vector.

In some embodiments, a single viral vector is used to deliver a nucleic acid encoding Cas9 and at least one gRNA to a cell. In some embodiments, Cas9 is provided to a cell using a first viral vector and at least one gRNA is provided to the cell using a second viral vector. In order to effect expression of sense or antisense gene constructs, the expression construct must be delivered into a cell. The cell may be a muscle cell, a satellite cell, a mesangioblast, a bone marrow derived cell, a stromal cell or a mesenchymal stem cell. In embodiments, the cell is a cardiac muscle cell, a skeletal muscle cell, or a smooth muscle cell. In embodiments, the cell is a cell in the tibialis anterior, quadriceps, soleus, diaphragm or heart. In some embodiments, the cell is an induced pluripotent stem cell (iPSC) or inner cell mass cell (iCM). In further embodiments, the cell is a human iPSC or a human iCM. In some embodiments, human iPSCs or human iCMs of the disclosure may be derived from a cultured stem cell line, an adult stem cell, a placental stem cell, or from another source of adult or embryonic stem cells that does not require the destruction of a human embryo. Delivery to a cell may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000™ is widely used and commercially available.

In certain embodiments, the liposome may be complexed with a hemagglutinating virus (HVJ) to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### X. DUCHENNE MUSCULAR DYSTROPHY

Duchenne muscular dystrophy (DMD) is a recessive X-linked form of muscular dystrophy, affecting around 1 in 5000 boys, which results in muscle degeneration and premature death. The disorder is caused by a mutation in the gene dystrophin (see GenBank Accession NO. NC_000023.11), located on the human X chromosome, which codes for the protein dystrophin (GenBank Accession No. AAA53189).

In humans, dystrophin mRNA contains 79 exons. Dystrophin mRNA is known to be alternatively spliced, resulting in various isoforms. Exemplary dystrophin isoforms are listed in Table 1.

The murine dystrophin protein has the following amino acid sequence (Uniprot Accession No. P11531, SEQ ID NO: 67):

```
      1 MWWVDCYRDV KKTTKWNASK GKHDNSDDGK RDGTGKKKGS TRVHANNVNK ARVKNNVDVN
    61 GSTDVDGNHK TGWNHWVKNV MKTMAGTNSK SWVRSTRNYV NVNTSSWSDG ANAHSHRDDW
  121 NSVVSHSATR HANAKCGKDD VATTYDKKSM YTSVVSAVMR TSSKVTRHHH MHYSTVSAGY
  181 TSSSKRKSYA TAAYVATSDS TSYSHARDKS DSSMTVNDSY TAVSWSADTR AGSNDVVKHA
  241 HGMMDTSHGV GNVGSVGKGK SDAVMNNSRW CRVASMKSKH KVMDNKKDDW TKTRTKKMGD
  301 DKCVHKVDVR VNSTHMVVVV DSSGDHATAA KVGDRWANCR WTDRWVDKWH TCSTWSKDAM
  361 KNTSGKDNMM SSHKSTKDKK KIMKSSNDSA KNKSVTKMWM NARWDNTKKS SASAVTTTST
  421 TTVMTVTMVT TRMVKHAKKR TVDSRKRDVD THSWTRSAVS SAVYRKGNSD KVNAARKAKR
  481 KDASRSAAVM ANGVNASRAS NSRWTCSRVN WYTNTYNMTT TANKTSTTST AKSKCKDVNR
  541 SAKSKKGGMD ADVATNHNHD GVRAKKTDTM RYTMSSRTWS SKSVYSVTYM RGKASSKNGN
  601 YSDTVKMAKK ASCKYSGHWK KSSVSCKHMN KRKNHKTKWM AVDVKWAGDA KKKCRVGDTS
  661 NSVNGGKKSA ASRTRNTWDH CRVYTRKAKA GDKTVSKDSM HWMTAYRDYK TDTAVMKRAK
  721 AKTKVKTTVN SVAHASAAKK TTTNYWCTRN GKCKTVWACW HSYKANKWNV KKTMNVAGTV
  781 SNMHHSNNRA TTDGGVMDNT NSRWRHAVRK KSSAKSHSDK AAYTDKVDAA MAKSDTSHSM
  841 KKHNGKDANR VSDVAKKDVS MKRKANRSKM DVKMHATKSV VSSHCVNYKS SVKSVMVKTG
  901 RVKKINKDRV TAKHYNGAKV TRKKCKSRKM RKMNVTWAAT DTTKRSAVGM SNDSVAWGKA
  961 TKKKAHKSVT GSKMVGKKTV DKSNSNWAVT SRVWNYKHMT DNTKWHADDS KKKKDKRKAM
1021 NDMRKVDSTR DAAKMANRGD HCRKVVSNRR AASHRKTGKA SKNSDKAGVN KDNKDMSDNG
1081 TVNRGDNRTD RKRKKTKHNA KDRSRRKKAS HWYYKRADDK CDKKASRDRK KDRKKKNAVR
1141 RAGSNGAAMA VISKRWRSNA RRNAHTHIMV VTTDMDVSYV STYTSHASVD HNTCAKDDKS
1201 KNKDNSGRDH KKKTAASATS MKVKVAVAMD GKHRMYKRGR DRSVKWRHHY DMKVNWNVKK
1261 TNNWHAKYKW YKDGGRAVVR TNATGSSKID VNKGSSRWHD CKARRKRKNV SRDNVWADNA
1321 TGDKVKARGK NIGGAVVSAR DKKKKTNWKV SRAKGVHKDR DHWSRNYNSA GDKVTVHGKA
1381 DVRSKGHYKK STVKRKDRSW AVNHRRTKDR AGSTTGASAS TVTVTSVVTK TVSKMSSVAA
1441 DNRAWTTDWS DRVKSRVMVG DDNMKKATDR RTAANKNKTS NARTTDRRWD VNRRNMKDST
1501 WAKAVGVRGK DSWKGHTVDA KKTTKAKDRR SVDVANDAKR DYSADDTRKV HMTNNTSWGN
1561 HKRVSAATHR DKSWTATTAN VDASRKKDSR GVRMKWDGTH TDYHNDNGKR SGSDARRDNM
1621 NKWSKKSNRS HASSDWKRHS VWKDDSRAGG DAVKNDHRAK RKTKVMSTTV RTGKYRRANV
1681 TRRKAVNAWD KNRSADWRKD ARAADDKRAV KGSWVGDDSD HKVKARGAKN VNRVNDAHTT
1741 GSYNSTDNTR WRVAVDRVRH AHRDGASHST SVGWRASNKV YYNHTTTCWD HKMTYSADNN
1801 VRSAYRTAMK RRKACDSSAA CDADHNKNDM DNCTTYDRHN NVNVCVDMCN WNVYDTGRTG
1861 RRVSKTGSCK AHDKYRYKVA SSTGCDRRGH DSRGVASGGS NSVRSCANNK AADWMRSMVW
1921 VHRVAAATAK HAKCNCKCGR YRSKHNYDCS CSGRVAKGHK MHYMVYCTTT SGDVRDAKVK
1981 NKRTKRYAKH RMGYVTVGDN MTVTNWVDSA ASSSHDDTHS RHYASRAMNS NGSYNDSSNS
2041 DDHHYCSNDS SRSASSRGRA DNRNAYDRKH HKGSSMMTSS RDAAAKRHKG RARMDHNKSH
2101 RRAAKVNGTT VSSSTSRSDS SMRVVGSTSS MGDSDTSTGV MNNSSSRGRN AGKMRDTM.
```

Dystrophin is an important component within muscle tissue that provides structural stability to the dystroglycan complex (DGC) of the cell membrane. While both sexes can carry the mutation, females are rarely affected with the skeletal muscle form of the disease.

Mutations vary in nature and frequency. Large genetic deletions are found in about 60-70% of cases, large duplications are found in about 10% of cases, and point mutants or other small changes account for about 15-30% of cases. Bladen et al. (2015), who examined some 7000 mutations, catalogued a total of 5,682 large mutations (80% of total mutations), of which 4,894 (86%) were deletions (1 exon or larger) and 784 (14%) were duplications (1 exon or larger). There were 1,445 small mutations (smaller than 1 exon, 20% of all mutations), of which 358 (25%) were small deletions and 132 (9%) small insertions, while 199 (14%) affected the splice sites. Point mutations totaled 756 (52% of small mutations) with 726 (50%) nonsense mutations and 30 (2%) missense mutations. Finally, 22 (0.3%) mid-intronic mutations were observed. In addition, mutations were identified within the database that would potentially benefit from novel genetic therapies for DMD including stop codon read-through therapies (10% of total mutations) and exon skipping therapy (80% of deletions and 55% of total mutations).

Symptoms usually appear in boys between the ages of 2 and 3 and may be visible in early infancy. Even though symptoms do not appear until early infancy, laboratory testing can identify children who carry the active mutation at birth. Progressive proximal muscle weakness of the legs and pelvis associated with loss of muscle mass is observed first. Eventually this weakness spreads to the arms, neck, and other areas. Early signs may include pseudohypertrophy (enlargement of calf and deltoid muscles), low endurance, and difficulties in standing unaided or inability to ascend staircases. As the condition progresses, muscle tissue experiences wasting and is eventually replaced by fat and fibrotic tissue (fibrosis). By age 10, braces may be required to aid in walking, but most patients are wheelchair dependent by age 12. Later symptoms may include abnormal bone development that leads to skeletal deformities, including curvature of the spine. Due to progressive deterioration of muscle, loss of movement occurs, eventually leading to paralysis. Intellectual impairment may or may not be present but if present, does not progressively worsen as the child ages. The average life expectancy for males afflicted with DMD is around 25.

The main symptom of Duchenne muscular dystrophy, a progressive neuromuscular disorder, is muscle weakness associated with muscle wasting with the voluntary muscles being first affected, especially those of the hips, pelvic area, thighs, shoulders, and calves. Muscle weakness also occurs later, in the arms, neck, and other areas. Calves are often enlarged. Symptoms usually appear before age 6 and may appear in early infancy. Other physical symptoms are:
1. Awkward manner of walking, stepping, or running—(patients tend to walk on their forefeet, because of an increased calf muscle tone. Also, toe walking is a compensatory adaptation to knee extensor weakness.)
2. Frequent falls.
3. Fatigue.
4. Difficulty with motor skills (running, hopping, jumping).
5. Lumbar hyperlordosis, possibly leading to shortening of the hip-flexor muscles. This has an effect on overall posture and a manner of walking, stepping, or running.
6. Muscle contractures of Achilles tendon and hamstrings impair functionality because the muscle fibers shorten and fibrose in connective tissue.
7. Progressive difficulty walking.
8. Muscle fiber deformities.
9. Pseudohypertrophy (enlarging) of tongue and calf muscles. The muscle tissue is eventually replaced by fat and connective tissue, hence the term pseudohypertrophy.
10. Higher risk of neurobehavioral disorders (e.g., ADHD), learning disorders (dyslexia), and non-progressive weaknesses in specific cognitive skills (in particular short-term verbal memory), which are believed to be the result of absent or dysfunctional dystrophin in the brain.
11. Eventual loss of ability to walk (usually by the age of 12).
12. Skeletal deformities (including scoliosis in some cases).
13. Trouble getting up from lying or sitting position.

The condition can often be observed clinically from the moment the patient takes his first steps, and the ability to walk usually completely disintegrates between the time the boy is 9 to 12 years of age. Most men affected with DMD become essentially “paralyzed from the neck down” by the age of 21. Muscle wasting begins in the legs and pelvis, then progresses to the muscles of the shoulders and neck, followed by loss of arm muscles and respiratory muscles. Calf muscle enlargement (pseudohypertrophy) is quite obvious. Cardiomyopathy particularly (dilated cardiomyopathy) is common, but the development of congestive heart failure or arrhythmia (irregular heartbeat) is only occasional.

A positive Gowers' sign reflects the more severe impairment of the lower extremities muscles. The child helps himself to get up with upper extremities: first by rising to stand on his arms and knees, and then “walking” his hands up his legs to stand upright. Affected children usually tire more easily and have less overall strength than their peers. Creatine kinase (CPK-MM) levels in the bloodstream are extremely high. An electromyography (EMG) shows that weakness is caused by destruction of muscle tissue rather than by damage to nerves. Genetic testing can reveal genetic errors in the Xp21 gene. A muscle biopsy (immunohistochemistry or immunoblotting) or genetic test (blood test) confirms the absence of dystrophin, although improvements in genetic testing often make this unnecessary.

DMD patients may suffer from:
1. Abnormal heart muscle (cardiomyopathy).
2. Congestive heart failure or irregular heart rhythm (arrhythmia).
3. Deformities of the chest and back (scoliosis).
4. Enlarged muscles of the calves, buttocks, and shoulders (around age 4 or 5). These muscles are eventually replaced by fat and connective tissue (pseudohypertrophy).
5. Loss of muscle mass (atrophy).
6. Muscle contractures in the heels, legs.
7. Muscle deformities.
8. Respiratory disorders, including pneumonia and swallowing with food or fluid passing into the lungs (in late stages of the disease).

Duchenne muscular dystrophy (DMD) is caused by a mutation of the dystrophin gene at locus Xp21, located on the short arm of the X chromosome. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane). Alterations in calcium and signaling pathways cause water to enter into the mitochondria, which then burst.

In skeletal muscle dystrophy, mitochondrial dysfunction gives rise to an amplification of stress-induced cytosolic calcium signals and an amplification of stress-induced reactive-oxygen species (ROS) production. In a complex cascading process that involves several pathways and is not clearly understood, increased oxidative stress within the cell damages the sarcolemma and eventually results in the death of the cell. Muscle fibers undergo necrosis and are ultimately replaced with adipose and connective tissue.

DMD is inherited in an X-linked recessive pattern. Females will typically be carriers for the disease while males will be affected. Typically, a female carrier will be unaware they carry a mutation until they have an affected son. The son of a carrier mother has a 50% chance of inheriting the defective gene from his mother. The daughter of a carrier mother has a 50% chance of being a carrier and a 50% chance of having two normal copies of the gene. In all cases, an unaffected father will either pass a normal Y to his son or a normal X to his daughter. Female carriers of an X-linked recessive condition, such as DMD, can show symptoms depending on their pattern of X-inactivation.

Exon deletions preceding exon 51 of the human DMD gene, which disrupt the open reading frame (ORF) by juxtaposing out of frame exons, represent the most common type of human DMD mutation. Skipping of exon 51 can, in principle, restore the DMD ORF in 13% of DMD patients with exon deletions.

Duchenne muscular dystrophy has an incidence of 1 in 5000 male infants. Mutations within the dystrophin gene can either be inherited or occur spontaneously during germline transmission. A table of exemplary but non-limiting mutations and corresponding models are set forth below:

TABLE 2
Dystrophin mutations and corresponding mouse models

| Deletion, small insertion and | |
|---|---|
| nonsense mutations | Name of Mouse Model |
| Exon 50 | ΔEx50 |
| Exon 44 | ΔEx44 |
| Exon 52 | ΔEx52 |
| Exon 43 | ΔEx43 |
| Exon 45 | ΔEx45 |

DMD Diagnosis: Genetic counseling is advised for people with a family history of the disorder. Duchenne muscular dystrophy can be detected with about 95% accuracy by genetic studies performed during pregnancy.

DNA test. The muscle-specific isoform of the dystrophin gene is composed of 79 exons, and DNA testing and analysis can usually identify the specific type of mutation of the exon or exons that are affected. DNA testing confirms the diagnosis in most cases.

Muscle biopsy. If DNA testing fails to find the mutation, a muscle biopsy test may be performed. A small sample of muscle tissue is extracted (usually with a scalpel instead of a needle) and a dye is applied that reveals the presence of dystrophin. Complete absence of the protein indicates the condition. As DNA tests have been developed that detect more of the many mutations that cause the condition, muscle biopsy is not required as often to confirm the presence of Duchenne's.

Prenatal tests. DMD is carried by an X-linked recessive gene. Males have only one X chromosome, so one copy of the mutated gene will cause DMD. Fathers cannot pass X-linked traits on to their sons, so the mutation is transmitted by the mother. If the mother is a carrier, and therefore one of her two X chromosomes has a DMD mutation, there is a 50% chance that a female child will inherit that mutation as one of her two X chromosomes and be a carrier. There is a 50% chance that a male child will inherit that mutation as his one X chromosome, and therefore have DMD.

Prenatal tests can tell whether their unborn child has the most common mutations. There are many mutations responsible for DMD, and some have not been identified, so genetic testing only works when family members with DMD have a mutation that has been identified. Prior to invasive testing, determination of the fetal sex is important; while males are sometimes affected by this X-linked disease, female DMD is extremely rare. This can be achieved by ultrasound scan at 16 weeks or more recently by free fetal DNA testing. Chorion villus sampling (CVS) can be done at 11-14 weeks, and has a 1% risk of miscarriage. Amniocentesis can be done after 15 weeks, and has a 0.5% risk of miscarriage. Fetal blood sampling can be done at about 18 weeks. Another option in the case of unclear genetic test results is fetal muscle biopsy.

Prognosis. Duchenne muscular dystrophy is a progressive disease which, left untreated, eventually affects all voluntary muscles and involves the heart and breathing muscles in later stages. The life expectancy is currently estimated to be around 25, but this varies from patient to patient. Recent advancements in medicine are extending the lives of those afflicted. The Muscular Dystrophy Campaign, which is a leading UK charity focusing on all muscle disease, states that “with high standards of medical care young men with Duchenne muscular dystrophy are often living well into their 30s.”

In rare cases, persons with DMD have been seen to survive into the forties or early fifties, with the use of proper positioning in wheelchairs and beds, ventilator support (via tracheostomy or mouthpiece), airway clearance, and heart medications, if required. Early planning of the required supports for later-life care has shown greater longevity in people living with DMD.

Curiously, in the mdx mouse model of Duchenne muscular dystrophy, the lack of dystrophin is associated with increased calcium levels and skeletal muscle myonecrosis. The intrinsic laryngeal muscles (ILM) are protected and do not undergo myonecrosis. ILM have a calcium regulation system profile suggestive of a better ability to handle calcium changes in comparison to other muscles, and this may provide a mechanistic insight for their unique pathophysiological properties. The ILM may facilitate the development of novel strategies for the prevention and treatment of muscle wasting in a variety of clinical scenarios.

### XI. COMBINATION THERAPIES

Beyond the gene therapy disclosed herein, there is no current cure for DMD. Prior to the development of the gene therapy of the disclosure, treatment was generally aimed at controlling the onset of symptoms to maximize the quality of life. These therapies may be used in combination with the gene therapies of the disclosure and may include the following:
1. Corticosteroids such as prednisolone and deflazacort increase energy and strength and defer severity of some symptoms.
2. Randomized control trials have shown that beta-2-agonists increase muscle strength but do not modify disease progression. Follow-up time for most RCTs on beta2-agonists is only around 12 months and hence results cannot be extrapolated beyond that time frame.
3. Mild, non-jarring physical activity such as swimming is encouraged. Inactivity (such as bed rest) can worsen the muscle disease.
4. Physical therapy is helpful to maintain muscle strength, flexibility, and function.
5. Orthopedic appliances (such as braces and wheelchairs) may improve mobility and the ability for self-care. Form-fitting removable leg braces that hold the ankle in place during sleep can defer the onset of contractures.
6. Appropriate respiratory support as the disease progresses is important.

Comprehensive multi-disciplinary care standards/guidelines for DMD have been developed by the Centers for Disease Control and Prevention (CDC), and are available at treat-nmd.eu/dmd/care/diagnosis-management-DMD.

DMD generally progresses through five stages, as outlined in Bushby et al., Lancet Neurol., 9(1): 77-93 (2010) and Bushby et al., Lancet Neurol., 9(2): 177-198 (2010), incorporated by reference in their entireties. During the presymptomatic stage, patients typically show developmental delay, but no gait disturbance. During the early ambulatory stage, patients typically show the Gowers' sign, waddling gait, and toe walking. During the late ambulatory stage, patients typically exhibit an increasingly labored gait and begin to lose the ability to climb stairs and rise from the floor. During the early non-ambulatory stage, patients are typically able to self-propel for some time, are able to maintain posture, and may develop scoliosis. During the late non-ambulatory stage, upper limb function and postural maintenance is increasingly limited.

In some embodiments, treatment is initiated in the presymptomatic stage of the disease. In some embodiments, treatment is initiated in the early ambulatory stage. In some embodiments, treatment is initiated in the late ambulatory stage. In embodiments, treatment is initiated during the early non-ambulatory stage. In embodiments, treatment is initiated during the late non-ambulatory stage.

Physical therapy. Physical therapists are concerned with enabling patients to reach their maximum physical potential. Their aim is to:
1. minimize the development of contractures and deformity by developing a program of stretches and exercises where appropriate,
2. anticipate and minimize other secondary complications of a physical nature by recommending bracing and durable medical equipment, and
3. monitor respiratory function and advise on techniques to assist with breathing exercises and methods of clearing secretions.

Respiration assistance. Modern “volume ventilators/respirators,” which deliver an adjustable volume (amount) of air to the person with each breath, are valuable in the treatment of people with muscular dystrophy related respiratory problems. The ventilator may require an invasive endotracheal or tracheotomy tube through which air is directly delivered, but, for some people non-invasive delivery through a face mask or mouthpiece is sufficient. Positive airway pressure machines, particularly bi-level ones, are sometimes used in this latter way. The respiratory equipment may easily fit on a ventilator tray on the bottom or back of a power wheelchair with an external battery for portability.

Ventilator treatment may start in the mid to late teens when the respiratory muscles can begin to collapse. If the vital capacity has dropped below 40% of normal, a volume ventilator/respirator may be used during sleeping hours, a time when the person is most likely to be under ventilating (“hypoventilating”). Hypoventilation during sleep is determined by a thorough history of sleep disorder with an oximetry study and a capillary blood gas (See Pulmonary Function Testing). A cough assist device can help with excess mucus in lungs by hyperinflation of the lungs with positive air pressure, then negative pressure to get the mucus up. If the vital capacity continues to decline to less than 30 percent of normal, a volume ventilator/respirator may also be needed during the day for more assistance. The person gradually will increase the amount of time using the ventilator/respirator during the day as needed.

### XII. MODELS OF DMD

### A. Humanized Mouse Models of DMD

### ΔEx50 Mouse

Rationale. The most common hot spot mutation region in DMD patients is the region between exon 45 to 51. Refraining and/or skipping of exon 51 may be used to treat the largest group (13-14%) of patients.

Recapitulation of the Human Condition. To investigate CRISPR/Cas9-mediated exon 51 skipping in vivo, a mouse model was generated that mimics the human “hot spot” region by deleting exon 50 using the CRISPR/Cas9 system directed by 2 sgRNAs. The deletion of exon 50 was confirmed by DNA sequencing. Deletion of exon 50 placed the dystrophin gene out of frame leading to the absence of dystrophin protein in skeletal muscle and heart. Mice lacking exon 50 showed pronounced dystrophic muscle changes by 2 months of age. Serum analysis of delta-exon 50 mice showed a significant increase in creatine kinase (CK) levels, indicative of muscle damage. Taken together, dystrophin protein expression, muscle histology and serum CK levels validated the dystrophic phenotype of the ΔEx50 mouse model.

### ΔEx44 Mouse

Rationale. Mutations in the dystrophin gene cause Duchenne muscular dystrophy (DMD), which is characterized by lethal degeneration of cardiac and skeletal muscles. Mutations that delete exon 44 of the dystrophin gene represent one of the most common causes of DMD and can be corrected in ˜12% of patients by skipping surrounding exons, which restores the dystrophin open reading frame.

Recapitulation of the Human Condition. As described in greater detail in Example 2, ΔEx44 DMD mice were generated in the C57/BL6J background using the CRISPR/Cas9 system. Two sgRNAs specific to the intronic regions surrounding exon 44 of the mouse Dmd locus were cloned into vector PX458 (Addgene plasmid #48138) using the primers from Table 16.

Deletion of exon 44 was confirmed by RT-PCR analysis. Sequencing of the RT-PCR products using primers for sequences in exons 43 and 46 confirmed the removal of exon 44 in these mice. At 4 weeks of age, immunostaining of tibialis anterior (TA) muscle, diaphragm, and heart in the ΔEx44 DMD mice showed complete absence of dystrophin protein expression. Western blot analysis confirmed loss of dystrophin protein. Necrotic fibers, inflammatory infiltration, and regenerative fibers with centralized nuclei were observed in 4-week old ΔEx44 DMD mice, indicative of a severe muscular dystrophy phenotype. Serum creatine kinase levels in the ΔEx44 DMD mice were elevated 22-fold compared to WT littermates, similar to mdx mice, an established DMD mouse model.

Shear force generated during muscle contraction leads to muscle membrane tearing in muscle lacking dystrophin, eventually causing myofiber degeneration and muscle fibrosis. Fibrotic tissue increases muscle stiffness and compromises contractility of muscles. To further analyze muscle function of ΔEx44 DMD mice, maximal tetanic force was measured in the extensor digitorum longus (EDL) muscle ex vivo. Compared with WT littermates at 4 weeks of age, ΔEx44 DMD mice showed an about 50% decrease in the specific and absolute tetanic force in the EDL muscle. A similar decrease of muscle strength was observed by grip strength analysis in 8-week old ΔEx44 DMD mice.

### B. Dog Models of DMD

### ΔEx50 Dog

Rationale. The ΔE50-MD dog model harbors a missense mutation in the 5′ donor splice site of exon 50 that results in deletion of exon 50 ((Walmsley et al., 2010)). Thus, this represents an ideal canine model for the investigation of gene-editing as an approach to permanently correct the most common DMD mutations in humans.

Recapitulation of the Human Condition. Expression of Cas9 and a single guide RNA (sgRNA) targeting a genomic sequence adjacent to the intron-exon junction of exon 51, using adeno-associated virus serotype 9 (AAV9), creates reframing mutations and allows skipping of exon 51. This leads to highly efficient restoration of dystrophin expression in skeletal and cardiac muscles of these dogs. These results demonstrate for the first time the applicability of a relatively simple, but effectively permanent, gene editing strategy for preventing DMD progression in a large mammal.

### C. Human Cells

Rationale. Patient-derived induced pluripotent stem cells (iPSCs) were generated from a DMD patient lacking exon 44 of the dystrophin gene (DMD) and from the patient's brother as a heathy control. Deletion of exon 44 (ΔEx44) disrupts the open reading frame of dystrophin by causing splicing of exon 43 to exon 45 and introducing a premature termination codon. The reading frame can be restored using CRISPR/Cas9 gene editing by skipping exon 43, which allows splicing between exons 42 and 45, or by skipping exon 45, which allows splicing between exons 43 and 46. Alternatively, reframing of exon 43 or 45 can restore the protein reading frame by inserting one nucleotide (+3n+1 insertion) or deleting two nucleotides (+3n-2 deletion).

### D. Translation to Human Therapy

As shown by the animal models and human cell lines provided by the disclosure, the therapeutic or pharmaceutical compositions described herein comprising at least one gRNA and at least one nuclease specifically and selectively target mutations in the DMD gene, effectively induce breaks in the target sequence, and by a variety of mechanisms (including reframing and/or exon skipping depending on the DMD mutation targeted), restore DMD expression and function. The therapeutic and/or pharmaceutical compositions described herein induce a reframing of the DMD gene and exon skipping to restore DMD gene expression in each of the mouse models, dog model and human cells lines. Accordingly, the proof of concept studies provided in this disclosure recapitulate the in vivo activity of these compositions when used as a human therapeutic. The compositions described herein are shown in vivo to have no detectable off-target activity, demonstrating that these compositions not only efficacious, but also safe. Moreover, the compositions described herein are shown in vivo to have no detectable immune response from a host with a functional immune system, demonstrating that these compositions not only efficacious, but also well-tolerated. Data presented herein demonstrate the composition of the disclosure are efficacious for treating and/or curing late-staged or advanced DMD in individuals with substantial muscle deterioration and functional losses.

Dosages provided herein may be scaled to human adults or to children of various ages using known equivalents, for example, as shown below in Table A or B (Reproduced from “Guidance for Industry Estimating the Maximum Safe Starting Dose in Initial Clinical Trials for Therapeutics in Adult Healthy Volunteers”, U.S. Department of Health and Human Services, Food and Drug Administration, Center for Drug Evaluation and Research (CDER), July 2005, Pharmacology and Toxicology):

TABLE A
Conversion of Animal Doses to Human Equivalent
Doses Based on Body Surface Area

| | To Convert | | |
|---|---|---|---|
| | Animal Dose in | To Convert Animal Dose in mg/kg | |
| | mg/kg to Dose in | to HED^{a} in mg/kg, Either: | |
| | mg/m², Multiply | Divide | Multiply |
| Species | by kₘ | Animal Dose By | Animal Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel | 7 | 5.3 | 0.19 |
| monkey | | | |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula:
HED = animal dose in mg/kg × (animal weight in kg/human weight in kg)^{0.33}.
^{b}This kₘ value is provided for reference only since healthy children will rarely be volunteers fox phase 1 trials.
^{c}For example, cynomolgus, thesus, and stumptail.

TABLE B
Effect of Allometric Exponent on Conversion Factor^{a}

| | | | | | Ratio of |
|---|---|---|---|---|---|
| | Weight Range^{b} | Conversion Factors^{c} | | | 0.75 to |
| Species | (kg) | Standard | b = 0.67 | b = 0.75 | 0.67 |
| Mouse | 0.018-0.033 | 0.081 | 0.075 | 0.141 | 1.88 |
| Rat | 0.09-0.40 | 0.162 | 0.156 | 0.245 | 1.57 |
| Rabbit | 1.5-3 | 0.324 | 0.33 | 0.43 | 1.30 |
| Monkey | 1.5-4 | 0.324 | 0.37 | 0.47 | 1.27 |
| Dog | 6.5-13.0 | 0.541 | 0.53 | 0.62 | 1.17 |

^{a}conversion factor = (Wₐₙᵢₘₐₗ/Wₕᵤₘₐₙ)^{(1-b)}
^{b}human weight range used was 50-80 kg (110-176 lb)
^{c}mean conversion factor calculated across entire animal weight range and human weight range

TABLE 3
sgRNAs targeting sites in various human DMD Exons

| | | | SEQ ID | |
|---|---|---|---|---|
| ID sgRNA | Strand | sgRNA | NO: | PAM |
| Exon50-#1 | 3′ | TAGTGGTCAGTCCAGGAGCT | 68 | AGG |
| Exon50-#2 | 3′ | GCTCCAATAGTGGTCAGTCC | 69 | AGG |
| Exon50-#3 | 5′ | GCTCCTGGACTGACCACTAT | 70 | TGG |
| Exon50-#4 | 3′ | ATACTTACAGGCTCCAATAG | 71 | TGG |
| Exon50-#5 | 3′ | ATGGGATCCAGTATACTTAC | 72 | AGG |
| Exon50-#6 | 5′ | ATTGGAGCCTGTAAGTATAC | 73 | TGG |
| Exon51-#1 | 3′ | CAGAGTAACAGTCTGAGTAG | 74 | GAG |
| Exon51-#2 | 3′ | CACCAGAGTAACAGTCTGAG | 75 | TAG |
| Exon51-#3 | 3′ | TATTTTGGGTTTTTGCAAAA | 76 | AGG |
| Exon51-#4 | 3′ | AGTAGGAGCTAAAATATTTT | 77 | GGG |
| Exon51-#5 | 3′ | GAGTAGGAGCTAAAATATTT | 78 | TGG |
| Exon51-#6 | 3′ | ACCAGAGTAACAGTCTGAGT | 79 | AGG |
| Exon51-#7 | 5′ | TCCTACTCAGACTGTTACTC | 80 | TGG |
| Exon51-#8 | 5′ | TACTCTGGTGACACAACCTG | 81 | TGG |
| Exon51-#9 | 3′ | GCAGTTTCCTTAGTAACCAC | 82 | AGG |
| Exon51-#10 | 5′ | GACACAACCTGTGGTTACTA | 83 | AGG |
| Exon51-#11 | 3′ | TGTCACCAGAGTAACAGTCT | 84 | GAG |
| Exon51-#12 | 3′ | AGGTTGTGTCACCAGAGTAA | 85 | CAG |
| Exon51-#13 | 3′ | AACCACAGGTTGTGTCACCA | 86 | GAG |
| Exon51-#14 | 3′ | GTAACCACAGGTTGTGTCAC | 87 | CAG |
| Exon53-#1 | 5′ | ATTTATTTTTCCTTTTATTC | 88 | TAG |
| Exon53-#2 | 5′ | TTTCCTTTTATTCTAGTTGA | 89 | AAG |
| Exon53-#3 | 3′ | TGATTCTGAATTCTTTCAAC | 90 | TAG |
| Exon53-#4 | 3′ | AATTCTTTCAACTAGAATAA | 91 | AAG |
| Exon53-#6 | 5′ | TTATTCTAGTTGAAAGAATT | 92 | CAG |
| Exon53-#7 | 5′ | TAGTTGAAAGAATTCAGAAT | 93 | CAG |
| Exon53-#8 | 5′ | AATTCAGAATCAGTGGGATG | 94 | AAG |
| Exon53-#9 | 3′ | ATTCTTTCAACTAGAATAAA | 95 | AGG |
| Exon53-#10 | 5′ | TTGAAAGAATTCAGAATCAG | 96 | TGG |
| Exon53-#11 | 5′ | TGAAAGAATTCAGAATCAGT | 97 | GGG |
| Exon53-#12 | 3′ | ACTGTTGCCTCCGGTTCTGA | 98 | AGG |
| Exon44-#1 | 3′ | CAGATCTGTCAAATCGCCTG | 99 | CAG |
| Exon44-#2 | 3′ | AAAACGCCGCCATTTCTCAA | 100 | CAG |
| Exon44-#3 | 3′ | AGATCTGTCAAATCGCCTGC | 101 | AGG |
| Exon44-#4 | 3′ | TATGGATCAAGAAAAATAGA | 102 | TGG |
| Exon44-#5 | 3′ | CGCCTGCAGGTAAAAGCATA | 103 | TGG |
| Exon44-#6 | 5′ | ATCCATATGCTTTTACCTGC | 104 | AGG |
| Exon44-#8 | 5′ | TTGACAGATCTGTTGAGAAA | 105 | TGG |
| Exon44-#9 | 5′ | ACAGATCTGTTGAGAAATGG | 106 | CGG |
| Exon44-#11 | 5′ | GGCGATTTGACAGATCTGTT | 107 | GAG |
| Exon44-#13 | 5′ | GGCGTTTTCATTATGATATA | 108 | AAG |
| Exon44-#14 | 5′ | ATGATATAAAGATATTTAAT | 109 | CAG |
| Exon44-#15 | 5′ | GATATTTAATCAGTGGCTAA | 110 | CAG |
| Exon44-#16 | 5′ | ATTTAATCAGTGGCTAACAG | 111 | AAG |
| Exon44-#17 | 3′ | AGAAACTGTTCAGCTTCTGT | 112 | TAG |
| Exon44-#1 | 5′ | GGGAACATGCTAAATACAAA | 113 | TGG |
| Exon44-#2 | 5′ | TAAATACAAATGGTATCTTA | 114 | AGG |
| Exon43-#1 | 5′ | GTTTTAAAATTTTTATATTA | 115 | CAG |
| Exon43-#2 | 5′ | TTTTATATTACAGAATATAA | 116 | AAG |
| Exon43-#3 | 5′ | ATATTACAGAATATAAAAGA | 117 | TAG |
| Exon45-#1 | 3′ | GTTCCTGTAAGATACCAAAA | 118 | AGG |
| Exon45-#2 | 5′ | TTGCCTTTTTGGTATCTTAC | 119 | AGG |
| Exon45-#3 | 5′ | TGGTATCTTACAGGAACTCC | 120 | AGG |
| Exon45-#4 | 5′ | ATCTTACAGGAACTCCAGGA | 121 | TGG |
| Exon45-#5 | 3′ | GCCGCTGCCCAATGCCATCC | 122 | TGG |
| Exon45-#6 | 5′ | CAGGAACTCCAGGATGGCAT | 123 | TGG |
| Exon45-#7 | 5′ | AGGAACTCCAGGATGGCATT | 124 | GGG |
| Exon45-#8 | 5′ | TCCAGGATGGCATTGGGCAG | 125 | CGG |
| Exon45-#9 | 5′ | GTCAGAACATTGAATGCAAC | 126 | TGG |
| Exon45-#10 | 3′ | AGTTCCTGTAAGATACCAAA | 127 | AAG |
| Exon45-#11 | 3′ | TGCCATCCTGGAGTTCCTGT | 128 | AAG |
| Exon45-#12 | 5′ | TTGGTATCTTACAGGAACTC | 129 | CAG |
| Exon45-#13 | 3′ | CGCTGCCCAATGCCATCCTG | 130 | GAG |
| Exon45-#14 | 5′ | AACTCCAGGATGGCATTGGG | 131 | CAG |
| Exon45-#15 | 5′ | GGGCAGCGGCAAACTGTTGT | 132 | CAG |
| Exon46-#1 | 3′ | ATTCTTTTGTTCTTCTAGCC | 133 | TGG |
| Exon46-#2 | 3′ | TGCTCTTTTCCAGGTTCAAG | 134 | GGG |
| Exon46-#3 | 5′ | GCTAGTATCCCACTTGAACC | 135 | TGG |
| Exon46-#4 | 3′ | TTTAGTTGCTGCTCTTTTCC | 136 | TGG |
| Exon46-#5 | 5′ | AGAAAAGCTTGAGCAAGTCA | 137 | AGG |
| Exon52-#1 | 3′ | AGATCTGTCAAATCGCCTGC | 138 | AGG |
| Exon52-#2 | 3′ | AATCCTGCATTGTTGCCTGT | 139 | AAG |
| Exon52-#3 | 5′ | CGCTGAAGAACCCTGATACT | 140 | AAG |
| Exon52-#4 | 3′ | GAACAAATATCCCTTAGTAT | 141 | CAG |
| Exon52-#5 | 3′ | CTGTAAGAACAAATATCCCT | 142 | TAG |
| Exon52-#6 | 5′ | CTAAGGGATATTTGTTCTTA | 143 | CAG |
| Exon52-#8 | 5′ | TGTTCTTACAGGCAACAATG | 144 | CAG |
| Exon52-#9 | 5′ | CAACAATGCAGGATTTGGAA | 145 | CAG |
| Exon52-#10 | 5′ | ACAATGCAGGATTTGGAACA | 146 | GAG |
| Exon52-#11 | 5′ | ATTTGGAACAGAGGCGTCCC | 147 | CAG |
| Exon52-#12 | 5′ | ACAGAGGCGTCCCCAGTTGG | 148 | AAG |
| Exon2-#1 | 5′ | TATTTTTTTATTTTGCATTT | 149 | TAG |
| Exon2-#2 | 5′ | TTATTTTGCATTTTAGATGA | 150 | AAG |
| Exon2-#3 | 5′ | ATTTTGCATTTTAGATGAAA | 151 | GAG |
| Exon2-#4 | 5′ | TTGCATTTTAGATGAAAGAG | 152 | AAG |
| Exon2-#5 | 5′ | ATGAAAGAGAAGATGTTCAA | 153 | AAG |

TABLE 4
Additional gRNA targeting sequences

| | | | | | | SEQ | |
|---|---|---|---|---|---|---|---|
| | | | | | | ID | |
| Name | Species | Gene | Target | Strand | Sequence | NO | PAM |
| DCR1 | Human | DMD | Intron | + | attggctttgatttcccta | 154 | GGG |
| | | | 50 | | | | |
| DCR2 | Human | DMD | Intron | − | tgtagagtaagtcagccta | 155 | TGG |
| | | | 50 | | | | |
| DCR3 | Human | DMD | Exon | + | cctactcagactgttactc | 156 | TGG |
| | | | 51-55′ | | | | |
| DCR4 | Human | DMD | Exon | + | ttggacagaacttaccgac | 157 | TGG |
| | | | 51-53′ | | | | |
| DCR5 | Human | DMD | Intron | − | cagttgcctaagaactggt | 158 | GGG |
| | | | 51 | | | | |
| DCR6 | Human | DMD | Intron | − | GGGCTCCACCCTCACGAGT | 159 | GGG |
| | | | 44 | | | | |
| DCR7 | Human | DMD | Intron | + | TTTGCTTCGCTATAAAACG | 160 | AGG |
| | | | 55 | | | | |
| DCR8 | Human | DMD | Exon 41 | + | TCTGAGGATGGGGCCGCAA | 161 | TGG |
| DCR9 | Human | DMD | Exon 44 | − | GATCTGTCAAATCGCCTGC | 162 | AGG |
| DCR10 | Human | DMD | Exon 45 | + | CCAGGATGGCATTGGGCAG | 163 | CGG |
| DCR11 | Human | DMD | Exon 45 | + | CTGAATCTGCGGTGGCAGG | 164 | AGG |
| DCR12 | Human | DMD | Exon 46 | − | TTCTTTTGTTCTTCTAGCc | 165 | TGG |
| DCR13 | Human | DMD | Exon 46 | + | GAAAAGCTTGAGCAAGTCA | 166 | AGG |
| DCR14 | Human | DMD | Exon 47 | + | GAAGAGTTGCCCCTGCGCC | 167 | AGG |
| DCR15 | Human | DMD | Exon 47 | + | ACAAATCTCCAGTGGATAA | 168 | AGG |
| DCR16 | Human | DMD | Exon 48 | − | TGTTTCTCAGGTAAAGCTC | 169 | TGG |
| DCR17 | Human | DMD | Exon 48 | + | GAAGGACCATTTGACGTTa | 170 | AGG |
| DCR18 | Human | DMD | Exon 49 | − | AACTGCTATTTCAGTTTCc | 171 | TGG |
| DCR19 | Human | DMD | Exon 49 | + | CCAGCCACTCAGCCAGTGA | 172 | AGG |
| DCR20 | Human | DMD | Exon 50 | + | gtatgcttttctgttaaag | 173 | AGG |
| DCR21 | Human | DMD | Exon 50 | + | CTCCTGGACTGACCACTAT | 174 | TGG |
| DCR22 | Human | DMD | Exon 52 | + | GAACAGAGGCGTCCCCAGT | 175 | TGG |
| DCR23 | Human | DMD | Exon 52 | + | GAGGCTAGAACAATCATTA | 176 | CGG |
| DCR24 | Human | DMD | Exon 53 | + | ACAAGAACACCTTCAGAAC | 177 | CGG |
| DCR25 | Human | DMD | Exon 53 | − | GGTTTCTGTGATTTTCTTT | 178 | TGG |
| DCR26 | Human | DMD | Exon 54 | + | GGCCAAAGACCTCCGCCAG | 179 | TGG |
| DCR27 | Human | DMD | Exon 54 | + | TTGGAGAAGCATTCATAAA | 180 | AGG |
| DCR28 | Human | DMD | Exon 55 | − | TCGCTCACTCACCctgcaa | 181 | AGG |
| DCR29 | Human | DMD | Exon 55 | + | AAAAGAGCTGATGAAACAA | 182 | TGG |
| DCR30 | Human | DMD | 5′ | + | TAcACTTTTCaAAATGCTT | 183 | TGG |
| | | | UTR/Ex | | | | |
| | | | on 1 | | | | |
| DCR31 | Human | DMD | Exon 51 | + | gagatgatcatcaagcaga | 184 | AGG |
| DCR32 | Mouse | DMD | mdx | + | ctttgaaagagcaaTaaaa | 185 | TGG |
| DCR33 | Human | DMD | Intron | − | CACAAAAGTCAAATCGGAA | 186 | TGG |
| | | | 44 | | | | |
| DCR34 | Human | DMD | Intron | − | ATTTCAATATAAGATTCGG | 187 | AGG |
| | | | 44 | | | | |
| DCR35 | Human | DMD | Intron | − | CTTAAGCAATCCCGAACTC | 188 | TGG |
| | | | 55 | | | | |
| DCR36 | Human | DMD | Intron | − | CCTTCTTTATCCCCTATCG | 189 | AGG |
| | | | 55 | | | | |
| DCR48 | Human | DMD | Intron | − | TAGTGATCGTGGATACGAG | 190 | AGG |
| | | | 45 | | | | |
| DCR49 | Human | DMD | Intron | − | TACAGCCCTCGGTGTATAT | 191 | TGG |
| | | | 45 | | | | |
| DCR50 | Human | DMD | Intron | − | GGAAGGAATTAAGCCCGAA | 192 | TGG |
| | | | 52 | | | | |
| DCR51 | Human | DMD | Intron | − | GGAACAGCTTTCGTAGTTG | 193 | AGG |
| | | | 53 | | | | |
| DCR52 | Human | DMD | Intron | + | ATAAAGTCCAGTGTCGATC | 194 | AGG |
| | | | 54 | | | | |
| DCR53 | | | Intron | + | AAAACCAGAGCTTCGGTCA | 195 | AGG |
| | | | 54 | | | | |
| DCR1 | Human | DMD | Intron | + | attggctttgatttcccta | 196 | GGG |
| | | | 50 | | | | |
| DCR2 | Human | DMD | Intron | − | tgtagagtaagtcagccta | 197 | TGG |
| | | | 50 | | | | |
| DCR3 | Human | DMD | Exon | + | cctactcagactgttactc | 198 | TGG |
| | | | 51-5′ | | | | |
| DCR4 | Human | DMD | Exon | + | ttggacagaacttaccgac | 199 | TGG |
| | | | 51-3′ | | | | |
| DCR5 | Human | DMD | Intron | − | cagttgcctaagaactggt | 200 | GGG |
| | | | 51 | | | | |
| DCR6 | Human | DMD | Intron | − | GGGCTCCACCCTCACGAGT | 201 | GGG |
| | | | 44 | | | | |
| DCR7 | Human | DMD | Intron | + | TTTGCTTCGCTATAAAACG | 202 | AGG |
| | | | 55 | | | | |
| DCR8 | Human | DMD | Exon 41 | + | TCTGAGGATGGGGCCGCAA | 203 | TGG |
| DCR9 | Human | DMD | Exon 44 | − | GATCTGTCAAATCGCCTGC | 204 | AGG |
| DCR10 | Human | DMD | Exon 45 | + | CCAGGATGGCATTGGGCAG | 205 | CGG |
| DCR11 | Human | DMD | Exon 45 | + | CTGAATCTGCGGTGGCAGG | 206 | AGG |
| DCR12 | Human | DMD | Exon 46 | − | TTCTTTTGTTCTTCTAGCc | 207 | TGG |
| DCR13 | Human | DMD | Exon 46 | + | GAAAAGCTTGAGCAAGTCA | 208 | AGG |
| DCR14 | Human | DMD | Exon 47 | + | GAAGAGTTGCCCCTGCGCC | 209 | AGG |
| DCR15 | Human | DMD | Exon 47 | + | ACAAATCTCCAGTGGATAA | 210 | AGG |
| DCR16 | Human | DMD | Exon 48 | − | TGTTTCTCAGGTAAAGCTC | 211 | TGG |
| DCR17 | Human | DMD | Exon 48 | + | GAAGGACCATTTGACGTTa | 212 | AGG |
| DCR18 | Human | DMD | Exon 49 | − | AACTGCTATTTCAGTTTCc | 213 | TGG |
| DCR19 | Human | DMD | Exon 49 | + | CCAGCCACTCAGCCAGTGA | 214 | AGG |
| DCR20 | Human | DMD | Exon 50 | + | gtatgcttttctgttaaag | 215 | AGG |
| DCR21 | Human | DMD | Exon 50 | + | CTCCTGGACTGACCACTAT | 216 | TGG |
| DCR22 | Human | DMD | Exon 52 | + | GAACAGAGGCGTCCCCAGT | 217 | TGG |
| DCR23 | Human | DMD | Exon 52 | + | GAGGCTAGAACAATCATTA | 218 | CGG |
| DCR24 | Human | DMD | Exon 53 | + | ACAAGAACACCTTCAGAAC | 219 | CGG |
| DCR25 | Human | DMD | Exon 53 | − | GGTTTCTGTGATTTTCTTT | 220 | TGG |
| DCR26 | Human | DMD | Exon 54 | + | GGCCAAAGACCTCCGCCAG | 221 | TGG |
| DCR27 | Human | DMD | Exon 54 | + | TTGGAGAAGCATTCATAAA | 222 | AGG |
| DCR28 | Human | DMD | Exon 55 | − | TCGCTCACTCACCctgcaa | 223 | AGG |
| DCR29 | Human | DMD | Exon 55 | + | AAAAGAGCTGATGAAACAA | 224 | TGG |
| DCR30 | Human | DMD | 5′ | + | TAcACTTTTCaAAATGCTT | 225 | TGG |
| | | | UTR/Ex | | | | |
| | | | on 1 | | | | |
| DCR31 | Human | DMD | Exon 51 | + | gagatgatcatcaagcaga | 226 | AGG |
| DCR33 | Human | DMD | Intron | − | CACAAAAGTCAAATCGGAA | 227 | TGG |
| | | | 44 | | | | |
| DCR34 | Human | DMD | Intron | − | ATTTCAATATAAGATTCGG | 228 | AGG |
| | | | 44 | | | | |
| DCR35 | Human | DMD | Intron | − | CTTAAGCAATCCCGAACTC | 229 | TGG |
| | | | 55 | | | | |
| DCR36 | Human | DMD | Intron | − | CCTTCTTTATCCCCTATCG | 230 | AGG |
| | | | 55 | | | | |
| DCR48 | Human | DMD | Intron | − | TAGTGATCGTGGATACGAG | 231 | AGG |
| | | | 45 | | | | |
| DCR49 | Human | DMD | Intron | − | TACAGCCCTCGGTGTATAT | 232 | TGG |
| | | | 45 | | | | |
| DCR50 | Human | DMD | Intron | − | GGAAGGAATTAAGCCCGAA | 233 | TGG |
| | | | 52 | | | | |
| DCR51 | Human | DMD | Intron | − | GGAACAGCTTTCGTAGTTG | 234 | AGG |
| | | | 53 | | | | |
| DCR52 | Human | DMD | Intron | + | ATAAAGTCCAGTGTCGATC | 235 | AGG |
| | | | 54 | | | | |
| DCR53 | Human | DMD | Intron | + | AAAACCAGAGCTTCGGTCA | 236 | AGG |
| | | | 54 | | | | |

TABLE 5
Additional gRNA targeting sequences for human DMD
intron 52, exon 53, and intron 53

| | | | | | SEQ ID | |
|---|---|---|---|---|---|---|
| Species | Gene | Target | Strand | gRNA sequence | NO | PAM |
| Human | DMD | Intron 52 | + | GTTGCCTGGTATGTCTAGCC | 237 | TGT |
| Human | DMD | Exon 53 | + | AAGAACACCTTCAGAACCGG | 238 | AGG |
| Human | DMD | Intron 52 | + | GGTATGTCTAGCCTGTCACA | 239 | TGG |
| Human | DMD | Intron 52 | − | TGTGACAGGCTAGACATACC | 240 | AGG |
| Human | DMD | Intron 52 | + | GAGGCATCAGTTGCCTGGTA | 241 | TGT |
| Human | DMD | Exon 53 | − | GTTGCCTCCGGTTCTGAAGG | 242 | TGT |
| Human | DMD | Intron 52 | − | AGTAGTAAATGCTAGTCTGG | 243 | AGG |
| Human | DMD | Intron 52 | − | TATAGTAGTAAATGCTAGTC | 244 | TGG |
| Human | DMD | Exon 53 | − | TCCGGTTCTGAAGGTGTTCT | 245 | TGT |
| Human | DMD | Intron 52 | + | GAGGGGAGGCATCAGTTGCC | 246 | TGG |
| Human | DMD | Intron 52 | + | CATAAATGTGAGATAACGTT | 247 | TGG |
| Human | DMD | Intron 52 | + | GTTCTAGGGATCAAGAATTG | 248 | AGT |
| Human | DMD | Intron 52 | + | AGGGGAGGCATCAGTTGCCT | 249 | GGT |
| Human | DMD | Intron 52 | + | ATTATATACAGGCTGCCTCT | 250 | GGG |
| Human | DMD | Intron 52 | + | AGGGGATATGGATATTCTGC | 251 | TGT |
| Human | DMD | Intron 52 | + | TGTCTAGCCTGTCACATGGA | 252 | TGG |
| Human | DMD | Intron 52 | + | CATTATATACAGGCTGCCTC | 253 | TGG |
| Human | DMD | Intron 52 | − | ACCCATCAGCCTGATTTCCC | 254 | AGT |
| Human | DMD | Intron 52 | + | GGCAATGAAATGCTGGCACT | 255 | GGG |
| Human | DMD | Intron 52 | + | AATGTGAGATAACGTTTGGA | 256 | AGT |
| Human | DMD | Intron 52 | + | CACTGGGAAATCAGGCTGAT | 257 | GGG |
| Human | DMD | Exon 53 | + | TACAAGAACACCTTCAGAAC | 258 | CGG |
| Human | DMD | Intron 52 | − | GGAGTTTGCTTTGCCATCCA | 259 | TGT |
| Human | DMD | Intron 52 | + | GATGGCAAAGCAAACTCCTG | 260 | TGG |
| Human | DMD | Intron 52 | + | TGATGGGTGCTGAAGTGGCA | 261 | AGG |
| Human | DMD | Intron 52 | − | TGAAGGCTTTCTGTTGCCAC | 262 | AGG |
| Human | DMD | Intron 52 | − | TGATGGCAAATATTAGTTTC | 263 | TGT |
| Human | DMD | Intron 52 | − | CAAACGTTATCTCACATTTA | 264 | TGT |
| Human | DMD | Intron 52 | − | AGGCTTTCTGTTGCCACAGG | 265 | AGT |
| Human | DMD | Intron 52 | − | TAATGACTAATTGTAGGAAT | 266 | TGG |
| Human | DMD | Intron 52 | + | AGGCAATGAAATGCTGGCAC | 267 | TGG |
| Human | DMD | Intron 52 | − | AGAACCAAATATGAATCATC | 268 | TGT |
| Human | DMD | Intron 52 | + | GCCTTCAGGCAATGAAATGC | 269 | TGG |
| Human | DMD | Intron 52 | + | AATGCTGGCACTGGGAAATC | 270 | AGG |
| Human | DMD | Intron 52 | − | TGCTTTGCCATCCATGTGAC | 271 | AGG |
| Human | DMD | Intron 52 | − | GCCAGCATTTCATTGCCTGA | 272 | AGG |
| Human | DMD | Intron 52 | + | ACTGGGAAATCAGGCTGATG | 273 | GGT |
| Human | DMD | Intron 52 | + | TGATGTTCTATGTCACCTTC | 274 | AGG |
| Human | DMD | Intron 52 | + | TGTGGCAACAGAAAGCCTTC | 275 | AGG |
| Human | DMD | Intron 52 | + | TCTAACAGATGATTCATATT | 276 | TGG |
| Human | DMD | Intron 52 | − | TTATTCATTGTGTTATGGCT | 277 | AGG |
| Human | DMD | Intron 52 | − | GCCTGTATATAATGACTAAT | 278 | TGT |
| Human | DMD | Intron 52 | + | TACAATTAGTCATTATATAC | 279 | AGG |
| Human | DMD | Intron 52 | + | GCACTGGGAAATCAGGCTGA | 280 | TGG |
| Human | DMD | Intron 52 | − | TGTATATAATGACTAATTGT | 281 | AGG |
| Human | DMD | Intron 52 | + | GTGCTGAAGTGGCAAGGATG | 282 | AGG |
| Human | DMD | Intron 52 | + | GATGATTCATATTTGGTTCT | 283 | AGG |
| Human | DMD | Exon 53 | + | ATTCAGAATCAGTGGGATGA | 284 | AGT |
| Human | DMD | Intron 52 | + | TGCTGAAGTGGCAAGGATGA | 285 | GGG |
| Human | DMD | Intron 52 | + | ATGATTCATATTTGGTTCTA | 286 | GGG |
| Human | DMD | Intron 52 | + | GCAACATAAATGTGAGATAA | 287 | CGT |
| Human | DMD | Intron 52 | − | TGGCTAGGATGATGAACAAC | 288 | AGG |
| Human | DMD | Intron 52 | + | TGGATGGCAAAGCAAACTCC | 289 | TGT |
| Human | DMD | Intron 52 | + | GTGGCAAGGATGAGGGGATA | 290 | TGG |
| Human | DMD | Intron 52 | + | CTAACAGATGATTCATATTT | 291 | GGT |
| Human | DMD | Exon 53 | + | TGAAAGAATTCAGAATCAGT | 292 | GGG |
| Human | DMD | Intron 52 | + | GGAGCTGGAGGGGAGGCATC | 293 | AGT |
| Human | DMD | Intron 52 | + | GGATATGGATATTCTGCTGT | 294 | AGT |
| Human | DMD | Intron 52 | + | CAGGCTGATGGGTGCTGAAG | 295 | TGG |
| Human | DMD | Exon 53 | + | AGTTGAAAGAATTCAGAATC | 296 | AGT |
| Human | DMD | Intron 52 | − | TTCATTGCCTGAAGGCTTTC | 297 | TGT |
| Human | DMD | Intron 52 | + | GCTGAAGTGGCAAGGATGAG | 298 | GGG |
| Human | DMD | Exon 53 | + | TTGAAAGAATTCAGAATCAG | 299 | TGG |
| Human | DMD | Intron 52 | + | TACAGGCTGCCTCTGGGAGC | 300 | TGG |
| Human | DMD | Intron 52 | + | ATATATATGTTGATGTTCTA | 301 | TGT |
| Human | DMD | Intron 52 | − | AATATATAGTAGTAAATGCT | 302 | AGT |
| Human | DMD | Intron 52 | + | GCTGCCTCTGGGAGCTGGAG | 303 | GGG |
| Human | DMD | Intron 52 | + | AGGCTGCCTCTGGGAGCTGG | 304 | AGG |
| Human | DMD | Intron 52 | − | CTTTTTTGATGGCAAATATT | 305 | AGT |
| Human | DMD | Intron 52 | + | GCCTCCCCTCCAGCTCCCAG | 306 | AGG |
| Human | DMD | Intron 52 | + | GCCTCTGGGAGCTGGAGGGG | 307 | AGG |
| Human | DMD | Intron 52 | + | ATCAGGCTGATGGGTGCTGA | 308 | AGT |
| Human | DMD | Intron 52 | − | TTTTAAATGTAACTTCCAAA | 309 | CGT |
| Human | DMD | Intron 52 | − | CTGTTAAATTATTTTCCTGA | 310 | AGG |
| Human | DMD | Intron 52 | + | TCCAGCTCCCAGAGGCAGCC | 311 | TGT |
| Human | DMD | Intron 52 | + | GGCTGCCTCTGGGAGCTGGA | 312 | CGG |
| Human | DMD | Intron 52 | − | TCTGGAGGAGACATTTTAAA | 313 | TGT |
| Human | DMD | Intron | − | ATTCTTTCAACTAGAATAAA | 314 | AGG |
| | | 52/Exon 53 | | | | |
| Human | DMD | Intron 52 | − | TGTTAAATTATTTTCCTGAA | 315 | GGT |
| Human | DMD | Intron 52 | + | AAAAAAGCAAAGAATCCTGT | 316 | TGT |
| Human | DMD | Intron 52 | − | AAAAATTATTCATTGTGTTA | 317 | TGG |
| Human | DMD | Intron 52 | + | TTTTAAATAAGCAACATAAA | 318 | TGT |
| Human | DMD | Intron 52 | + | AAATTTTATATATATGTTGA | 319 | TGT |
| Human | DMD | Intron 52 | − | AGGAAAAATAAATATATAGT | 320 | AGT |
| Human | DMD | Intron 52 | − | ATTTAAAAAATTATTCATTG | 321 | TGT |
| Human | DMD | Intron 52 | + | GAGTTAAAATTTTATATATA | 322 | TGT |
| Human | DMD | Intron 52 | + | TTTGGAAGTTACATTTAAAA | 323 | TGT |
| Human | DMD | Intron 52 | + | ATCAAAAAAGCAAAGAATCC | 324 | TGT |
| Human | DMD | Intron 52 | − | AGGATTCTTTGCTTTTTTGA | 325 | TGG |
| Human | DMD | Intron 52 | − | TTATTTAAAAAATTATTCAT | 326 | TGT |
| Human | DMD | Intron 52 | − | AAAAGGAAAAATAAATATAT | 327 | AGT |
| Human | DMD | Intron 52 | + | TTTATTTTTCCTTTTATTCT | 328 | AGT |
| Human | DMD | Exon 53 | + | TGGAAGGAGGGTCCCTATAC | 329 | AGT |
| Human | DMD | Intron 52 | − | GGTATCTTTGATACTAACCT | 330 | TGG |
| Human | DMD | Intron 53 | − | ACTGTTGCCTCCGGTTCTGA | 331 | AGG |
| Human | DMD | Exon 53 | + | CCTTCAGAACCGGAGGCAAC | 332 | AGT |
| Human | DMD | Exon 53 | + | AGCTGAGCAGGTCTTAGGAC | 333 | AGG |
| Human | DMD | Exon 53 | − | CTGTTGCCTCCGGTTCTGAA | 334 | GGT |
| Human | DMD | Exon 53 | + | CCAGAGCCAAGCTTGAGTCA | 335 | TGG |
| Human | DMD | Intron 53 | − | TCAGCTTTAACGTGATTTTC | 336 | TGT |
| Human | DMD | Exon 53 | − | CTCCTTCCATGACTCAAGCT | 337 | TGG |
| Human | DMD | Exon 53 | − | TTGGATTGCATCTACTGTAT | 338 | AGG |
| Human | DMD | Intron 53 | + | GCTCACTGAGCCTTGAACTC | 339 | TGG |
| Human | DMD | Exon 53 | − | ACTCAAGCTTGGCTCTGGCC | 340 | TGT |
| Human | DMD | Exon 53 | − | TGGATTGCATCTACTGTATA | 341 | GGG |
| Human | DMD | Exon 53 | + | ACAGGCCAGAGCCAAGCTTG | 342 | AGT |
| Human | DMD | Exon 53 | − | TTCTTTTGGATTGCATCTAC | 343 | TGT |
| Human | DMD | Exon 53 | − | TTCATTCAACTGTTGCCTCC | 344 | GGT |
| Human | DMD | Exon 53 | − | CCATGACTCAAGCTTGGCTC | 345 | TGG |
| Human | DMD | Intron 53 | − | GTATCTTTGATACTAACCTT | 346 | GGT |
| Human | DMD | Intron 53 | + | CTGAAATGAACAGTAGACTT | 347 | TGT |
| Human | DMD | Exon 53 | + | CAAGCTTGAGTCATGGAAGG | 348 | AGG |
| Human | DMD | Exon 53 | − | TTTCATTCAACTGTTGCCTC | 349 | CGG |
| Human | DMD | Exon 53 | − | TCCTTAGCTTCCAGCCATTG | 350 | TGT |
| Human | DMD | Exon 53 | − | CTTCCTTAGCTTCCAGCCAT | 351 | TGT |
| Human | DMD | Exon 53 | + | AAAGGATTCAACACAATGGC | 352 | TGG |
| Human | DMD | Exon 53 | + | AACACAATGGCTGGAAGCTA | 353 | AGG |
| Human | DMD | Intron 53 | + | TCACTGAGCCTTGAACTCTG | 354 | GGG |
| Human | DMD | Exon 53 | + | TGTTAAAGGATTCAACACAA | 355 | TGG |
| Human | DMD | Intron 53 | − | CATCATTAAATTACAATCTA | 356 | TGG |
| Human | DMD | Exon 53 | + | GAAGAAGCTGAGCAGGTCTT | 357 | AGG |
| Human | DMD | Exon 53 | + | AAATCACAGAAACCAAGGTT | 358 | AGT |
| Human | DMD | Exon 53 | + | AGCTTGAGTCATGGAAGGAG | 359 | GGT |
| Human | DMD | Exon 53 | − | CCTTTAACATTTCATTCAAC | 360 | TGT |
| Human | DMD | Exon 53 | − | ACTGTTCATTTCAGCTTTAA | 361 | CGT |
| Human | DMD | Exon 53 | + | AGCCAAGCTTGAGTCATGGA | 362 | AGG |
| Human | DMD | Intron 53 | + | ACGTTAAAGCTGAAATGAAC | 363 | AGT |
| Human | DMD | Exon 53 | + | GAGGCAACAGTTGAATGAAA | 364 | TGT |
| Human | DMD | Intron 53 | + | GATAAAATTATACCATAGAT | 365 | TGT |
| Human | DMD | Exon 53 | + | AAGCTTGAGTCATGGAAGGA | 366 | GGG |
| Human | DMD | Intron 53 | − | ATCATTAAATTACAATCTAT | 367 | GGT |
| Human | DMD | Intron 53 | + | CATAGATTGTAATTTAATGA | 368 | TGT |
| Human | DMD | Intron 53 | − | TAATTTTATCAAATGTAACC | 369 | AGT |
| Human | DMD | Intron 53 | − | AATAAATATACAAAGTCTAC | 370 | TGT |
| Human | DMD | Exon 53 | + | GCTAAGGAAGAAGCTGAGCA | 371 | GGT |
| Human | DMD | Exon 53 | + | AGCTAAGGAAGAAGCTGAGC | 372 | AGG |
| Human | DMD | Intron 53 | + | CTCACTGAGCCTTGAACTCT | 373 | GGG |
| Human | DMD | Exon 53 | − | ACAGTTGAATGAAATGTTAA | 374 | AGG |
| Human | DMD | Exon 53/ | − | TTGATACTAACCTTGGTTTC | 375 | TGT |
| | | Intron 53 | | | | |
| Human | DMD | Intron 53 | + | AAGTTATTAACAGAAAATCA | 376 | CGT |
| Human | DMD | Intron 53 | − | AGCCCCAGAGTTCAAGGCTC | 377 | AGT |
| Human | DMD | Intron 53 | − | TATGGTATAATTTTATCAAA | 378 | TGT |
| Human | DMD | Intron 53 | + | TGTAATTTAATGATGTTTAA | 379 | TGT |
| Human | DMD | Intron 53 | + | TTTAATGATGTTTAATGTAA | 380 | AGT |
| Human | DMD | Exon 53 | − | TGGTTTCTGTGATTTTCTTT | 381 | TGG |
| Human | DMD | Exon 53 | + | AAAGAAAATCACAGAAACCA | 382 | AGG |
| Human | DMD | Exon 53 | + | AAGAAAATCACAGAAACCAA | 383 | GGT |
| Human | DMD | Intron 53 | + | ACCTTTTTAAAATAAAATAC | 384 | TGG |
| Human | DMD | Intron 53 | + | CCTTTTTAAAATAAAATACT | 385 | GGT |
| Human | DMD | Intron 53 | − | CCAGTATTTTATTTTAAAAA | 386 | GGT |
| Human | DMD | Intron 53 | − | ACCAGTATTTTATTTTAAAA | 387 | AGG |
| Human | DMD | Intron 53 | − | AAGGCTCAGTGAGCTATGAT | 388 | TGT |
| Human | DMD | Intron 53 | − | CTAAGAAAATAAATATACAA | 389 | AGT |

### XIII. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

LRVQR-SpCas9 vector cloning and in vitro sgRNA screening. The LRVQR-SpCas9 was synthesized as gBlocks (Integrated DNA Technologies), and subcloned into a plasmid containing U6-sgRNA expression cassette, generating the pSpCas9-LRVQR-2A-GFP plasmid. CRISPR sgRNAs targeting human exon 53 were subcloned into the newly designed pSpCas9-LRVQR-2A-GFP plasmid, using BbsI digestion and T4 ligation, in vitro sgRNA screening was performed in human 293T cells. 1 million cells were transiently transfected with 2500 ng of pSpCas9-LRVQR-2A-GFP-U6-sgRNA plasmid using Lipofectamine 2000 transfection reagent according to the manufacturer's protocol. 3-day post transfection, GFP-positive 293T cells were enriched by fluorescence-activated cell sorting (FACS).

Human iPSC maintenance, nucleofection, and differentiation. Human iPSCs were cultured in mTeSR plus medium (STEMCELL Technologies) and passaged once reaching 70% confluence (1:18 split ratio). One hour before nucleofection, DMD ΔEx52 iPSCs were pretreated with 10 μM ROCK inhibitor (Y-27632) and dissociated into single cells using Accutase (Innovative Cell Technologies Inc.). DMD ΔEx52 iPSCs (1×10⁶) were mixed with 5 μg of the pSpCas9-LRVQR-2A-GFP-U6-sgRNA plasmid, and then nucleofected with the P3 Primary Cell 4D-Nucleofector X Kit (Lonza) according to the manufacturer's protocol. After nucleofection, DMD ΔEx52 iPSCs were cultured in mTeSR plus medium supplemented with 10 μM ROCK inhibitor, and Primocin (100 μg/ml; InvivoGen). Three days after nucleofection, GFP(+) cells were sorted by FACS and subjected to differentiation to cardiomyocytes and sequencing analysis.

Dystrophin Western blot. For Western blot of iPSC-derived cardiomyocytes, 2 million cardiomyocytes were harvested and lysed with lysis buffer (10% SDS, 62.5 mM Tris [pH 6.8], 1 mM EDTA, and protease inhibitor). Cell or tissue lysates were passed through a 25G syringe and then a 27G syringe, 10 times each. Protein concentration was determined by bicinchonic acid assay (BCA) assay, and 50 mg total protein was loaded onto a 4%-20% acrylamide gel. Gels were run at 100 V for 15 min and switched to 200 V for 45 min followed by 1 h, 20 min transfer to a polyvinylidene fluoride (PVDF) membrane at 100 V in cold room. The blot was incubated with mouse anti-dystrophin antibody (MANDYS8, Sigma-Aldrich, D8168) at 4° C. overnight and then with goat anti-mouse horseradish peroxidase (HRP) antibody (Bio-Rad Laboratories) at room temperature for 1 h. The blot was developed using Western Blotting Luminol Reagent (Santa Cruz Biotechnology, sc-2048). The loading control was determined by blotting with mouse anti-vinculin antibody (Sigma-Aldrich, V9131).

Dystrophin immunostaining. iPSC-derived cardiomyocytes were fixed with acetone, blocked with serum cocktail [2% normal horse serum/2% normal donkey serum/0.2% bovine serum albumin (BSA)/phosphate-buffered saline (PBS)], and incubated with a dystrophin antibody (MANDYS8, 1:800; Sigma-Aldrich) and troponin I antibody (H170, 1:200; Santa Cruz Biotechnology) in 0.2% BSA/PBS. Following overnight incubation at 4 degrees, they were incubated with secondary antibodies [biotinylated horse anti-mouse immunoglobulinG (IgG), 1:200 (Vector Laboratories), and fluorescein-conjugated donkey anti-rabbit IgG, 1:50 (Jackson ImmunoResearch)] for 1 hour. Nuclei were counterstained with Hoechst 33342 (Molecular Probes).

Human iPSC maintenance, nucleofection, and differentiation. Stem cell work described in this patent has been conducted under the oversight of the UT Southwestern Stem Cell Research Oversight (SCRO) Committee. Human iPSCs were cultured in mTeSR plus medium (STEMCELL Technologies) and passaged once reaching 70% confluence (1:18 split ratio). One hour before nucleofection, DMD ΔEx52 iPSCs were pretreated with 10 μM ROCK inhibitor (Y-27632) and dissociated into single cells using Accutase (Innovative Cell Technologies Inc.). DMD ΔEx52 iPSCs (1×10⁶) were mixed with 5 μg of the pSpCas9-LRVQR-2A-GFP-U6-sgRNA plasmid, and then nucleofected with the P3 Primary Cell 4D-Nucleofector X Kit (Lonza) according to the manufacturer's protocol. After nucleofection, DMD ΔEx52 iPSCs were cultured in mTeSR plus medium supplemented with 10 μM ROCK inhibitor, and Primocin (100 μg/ml; InvivoGen). Three days after nucleofection, GFP(+) cells were sorted by FACS and subjected to differentiation to cardiomyocytes and TIDE analysis, as previously described (Min et al., 2020).

In vivo AAV delivery into Δ52;h53KI DMD mice. Postnatal day 4 Δ52;h53KI DMD mice (body weight ˜3 g) were injected intraperitoneally with 80 μl of saline or AAV9 containing ssAAV2/9-SpCas9-LRVQR (8×10¹³ vg/kg) and scAAV2/9-U6-M11-sgRNAs (8×10¹³ vg/kg) using an ultrafine BD insulin syringe (Becton Dickinson). Four weeks after systemic delivery, Δ52;h53KI DMD mice and h53KI control mice were dissected for physiological, biochemical and histological analysis. Animal work described in this manuscript has been approved and conducted under the oversight of the University of Texas Southwestern Institutional Animal Care and Use Committee.

Dystrophin immunocytochemistry and immunohistochemistry. Immunocytochemistry of iPSC-derived cardiomyocytes was performed as previously described (Zhang et al., 2017). In brief, iPSC-derived cardiomyocytes were fixed with acetone, blocked with serum cocktail [2% normal horse serum, 2% normal donkey serum, 0.2% bovine serum albumin (BSA) in phosphate-buffered saline (PBS)], and incubated with a dystrophin antibody (MANDYS8, 1:800; Sigma-Aldrich) and troponin I antibody (H170, 1:200; Santa Cruz Biotechnology) in 0.2% BSA/PBS. Following overnight incubation at 4° C., cardiomyocytes were incubated with secondary antibodies [biotinylated horse anti-mouse immunoglobulin G (IgG), 1:200 (Vector Laboratories), and fluorescein-conjugated donkey anti-rabbit IgG, 1:50 (Jackson ImmunoResearch)] for 1 hr. Nuclei were counterstained with DAPI (1:250; Sigma-Aldrich).

Immunohistochemistry of skeletal muscles and heart of Δ50;h51KI mice and h51KI control mice was performed as previously described (Min et al., 2019). In brief, skeletal muscles and heart of h51KI and Δ50;h51KI mice were cryosectioned into 8 μm transverse sections and delipidated in 1% Triton X-100 in PBS (pH 7.4). Following delipidation, sections were incubated with mouse IgG blocking reagent (M.O.M. Kit, Vector Laboratories), washed, and sequentially equilibrated with M.O.M diluent (600 uL of M.O.M Protein Concentrate stock solution to 7.5 mL of PBS, pH 7.4). Then sections were incubated with mouse anti-dystrophin primary antibody (MANDYS8, 1:800; Sigma-Aldrich) and rabbit anti-laminin primary antibody (L9393, 1:500; Sigma-Aldrich) dissolved in M.O.M. diluent at room temperature for 1 hour. Followed by PBS wash, sections were then incubated with Avidin D conjugated with FITC (1:250; Vector Laboratories), goat anti-rabbit IgG secondary antibody conjugated with Alexa Fluor 555 (1:500; ThermoFisher Scientific), and DAPI (1:250; Sigma-Aldrich) dissolved in M.O.M. diluent at room temperature for 30 min.

Dystrophin Western blot analysis. For Western blot analysis of iPSC-derived cardiomyocytes, 4×10⁶ cells were lysed in lysis buffer [10% SDS, 62.5 mM tris (pH 6.8), 1 mM EDTA, and protease inhibitor]. Heart and skeletal muscles of Δ50;h51KI mice and h51KI control mice were crushed into fine powder using a liquid-nitrogen-frozen crushing apparatus and lysed in the same lysis buffer as iPSC-derived cardiomyocytes. Protein concentration was determined by Pierce BCA Protein Assay Kit according to manufacturer's protocol. A total 40 μg of protein was loaded onto 4-20% Criterion™ TGX™ Precast Midi Protein Gel (Bio-Rad Laboratories). Gels were run at 80 V for 30 min, switched to 130 V for 2 hr, followed by a wet transfer to a polyvinylidene difluoride (PVDF) membrane at 100 V at 4° C. for 90 min. For dystrophin protein detection, the PVDF membrane was blocked in blocking buffer (5% w/v nonfat dry milk, 1×TBS, 0.1% Tween-20) at room temperature for 1 hr, and then incubated with mouse anti-dystrophin primary antibody (MANDYS8, 1:1000; Sigma-Aldrich) at 4° C. overnight, followed by incubation with goat anti-mouse IgG (H+L)-HRP secondary antibody (1:10000; Bio-Rad) at room temperature for 1 hr. For vinculin protein detection, the PVDF membrane was cleared with stripping buffer, blocked with blocking buffer at room temperature for 1 hr. Then the PVDF membrane was incubated with mouse anti-vinculin primary antibody (V9131, 1:1000; Sigma-Aldrich) at room temperature for 1 hr, followed by incubation with goat anti-mouse IgG (H+L)-HRP secondary antibody (1:10000; Bio-Rad) at room temperature for 1 hr. The PVDF membrane was developed by Western Blotting Luminol Reagent (Santa Cruz) according to manufacturer's protocol and imaged by digital imager (Bio-Rad).

Grip strength test and serum CK measurement. Forelimb muscle strength of 4-week old Δ52;h53KI DMD mice and h53KI control mice was assessed by the grip strength meter (Columbus Instruments). In brief, an individual mouse was weighed and lifted by the tail causing the forelimbs to grasp the pull-bar assembly connected to the grip strength meter. The mouse was drawn along a straight line leading away from the sensor until the grip is broken and the peak amount of force in grams was recorded. Measurement of forelimb grip strength of each mouse was repeated 5 times in a blinded experimental design. Serum CK was measured by the Metabolic Phenotyping Core at UT Southwestern Medical Center using the VITROS 250 Chemistry System in a blinded experimental design.

### Results

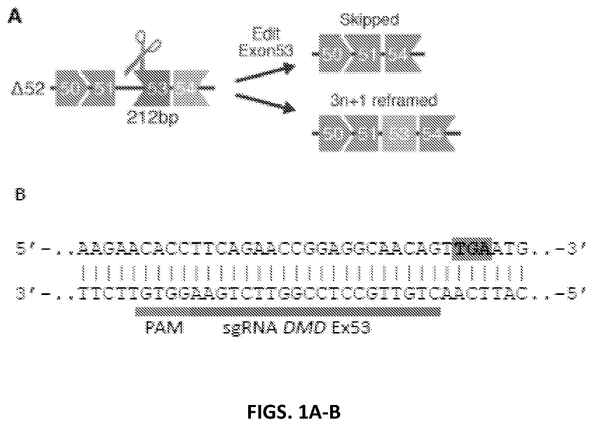

Strategies for LRVQR-SpCas9-mediated gene editing of human DMD exon 53. Human DMD exon 52 out-of-frame deletion results in splicing of exon 51 to exon 53, generating a premature termination codon in exon 53 (FIG. 1A). LRVQR-SpCas9-mediated single cut gene editing strategy was designed to restore the open reading frame of the dystrophin gene. Small insertions and deletions (INDELs) with one nucleotide insertion (3n+1) or two nucleotide deletions (3n-2) can reframe human exon 53. Large INDELs disrupting the 5′-AG-3′ splice acceptor sequence causes human exon 53 skipping, resulting in splicing of exon 51 to exon 54 (FIG. 1A). LRVQR-SpCas9 sgRNAs in human 293T cells were screened, and an optimal sgRNA with highly productive editing was identified. This optimized sgRNA recognizes a 5′-GGTG-3′ PAM in human exon 53 and cuts genomic DNA 18 bp upstream of the premature termination codon (FIG. 1B).

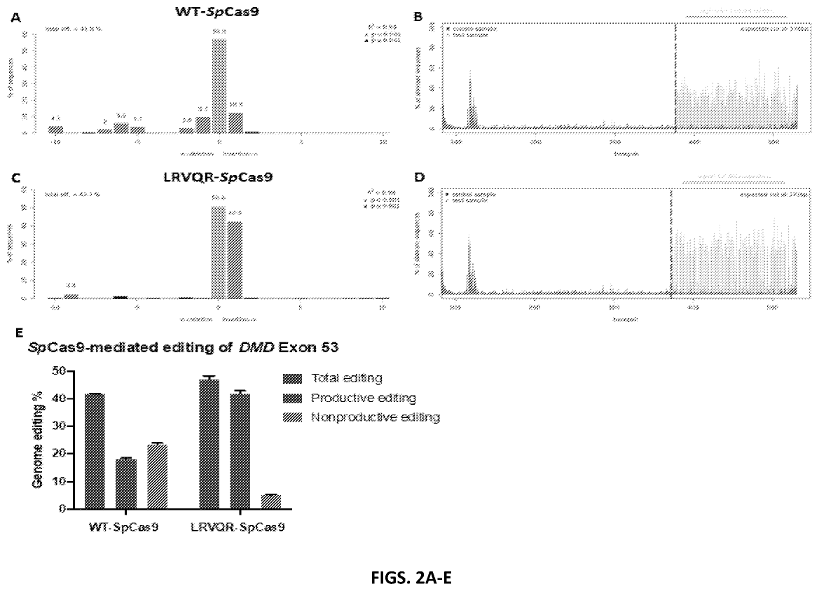

LRVQR-SpCas9 showed higher productive editing than the WT-SpCas9. Human 293T cells were transfected with LRVQR-SpCas9 or WT-SpCas9. WT-SpCas9 generated 41.8% of total genomic editing in human DMD exon 53 locus (FIGS. 2A-B). However, less than 20% of genomic editing events induced by WT-SpCas9 were productive editing (FIG. 2E). In contrast, LRVQR-SpCas9 generated 47.7% of total genomic editing in human DMD exon 53 locus (FIGS. 2C-D). Importantly, more than 40% of genomic editing events induced by LRVQR-SpCas9 were productive editing, which was significantly higher than WT-SpCas9 (FIG. 2E).

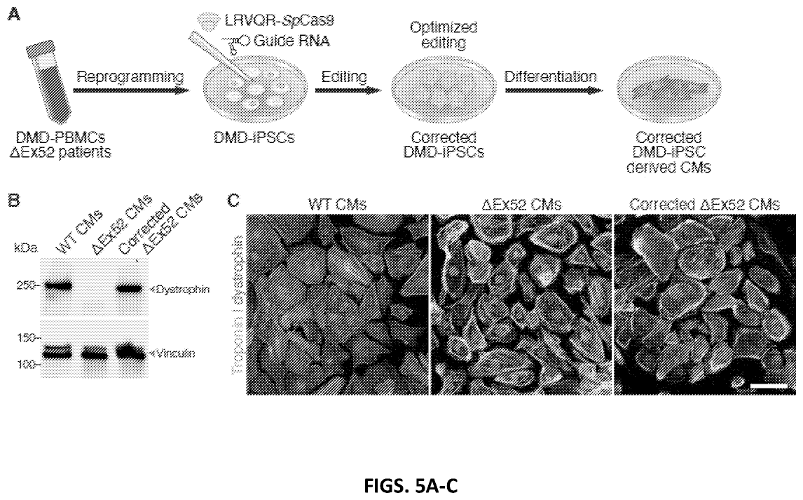

LRVQR-SpCas9 gene editing restores dystrophin expression in patient iPSC-derived ΔEx52 cardiomyocytes. LRVQR-SpCas9-mediated single-cut gene editing was performed in human iPSCs generated from a ΔEx52 DMD patient (FIG. 5A). This DMD iPSC line carries an out-of-frame deletion of DMD exon 52, which generates a premature termination codon in exon 53. The sgRNA that showed high productive editing was then used to correct the mutation in ΔEx52 iPSCs. From immunocytochemistry and Western blot analysis, it was determined that cardiomyocytes differentiated from LRVQR-SpCas9 gene edited ΔEx52 iPSC showed a high level of dystrophin protein expression comparable to healthy control cardiomyocytes, even without clonal selection and expansion (FIGS. 5B-C). It was thus concluded that LRVQR-SpCas9-mediated single-cut gene editing represents an efficient strategy to correct human exon 53 out-of-frame mutations in human DMD iPSCs.

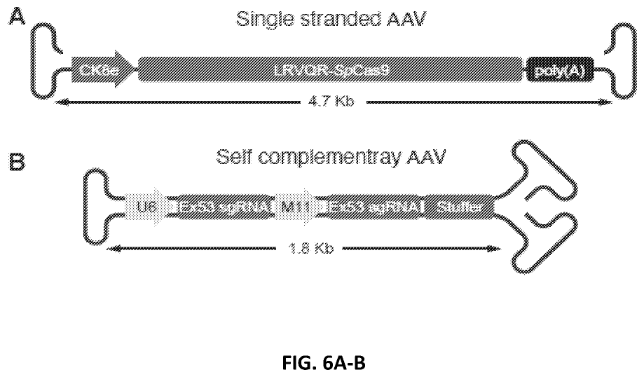

Design of AAV-based vectors to deliver CRISPR/LRVQR-SpCas9 gene editing components. To assess the efficacy of the LRVQR-SpCas9 nuclease and sgRNA in vivo, a dual AAV delivery system was designed to package the LRVQR-SpCas9 gene editing components individually. AAV serotype 9 (AAV9) was chosen because of its tropism to striated muscle. In this dual AAV delivery system, the LRVQR-SpCas9 nuclease was cloned into a single-stranded AAV vector and its expression was driven by the muscle-specific CK8e promoter (FIG. 6A). A double-stranded self-complementary AAV (scAAV) vector was chosen to express sgRNA because scAAV showed substantially higher viral transduction efficiency in systemic CRISPR gene editing (Min et al., 2020; Zhang et al., 2020). Moreover, two copies of the sgRNA expression cassette driven by two RNA polymerase III promoters, U6 and M11, were cloned because sgRNA has been shown to be rate-limiting for in vivo gene editing of DMD mouse models (FIG. 6B) (Hakim et al., 2018; Min et al., 2019).

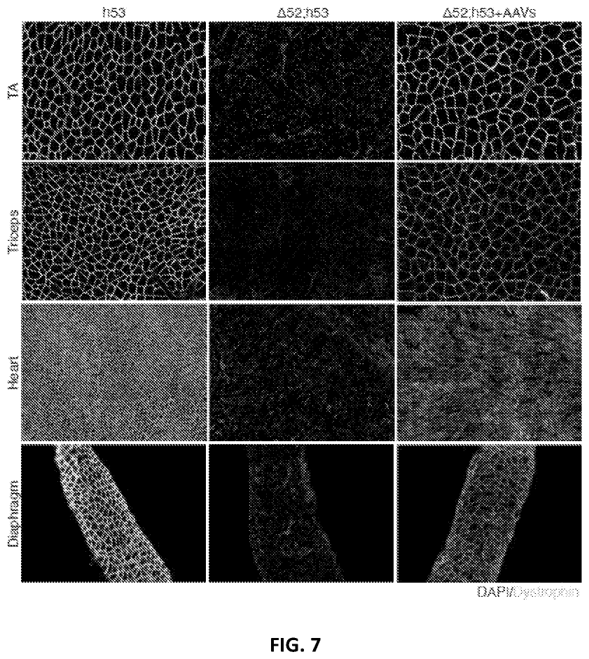

Systemic CRISPR/LRVQR-SpCas9 gene editing restores dystrophin expression in humanized DMD mice. To achieve whole body gene editing, AAV-SpCas9-LRVQR and AAV-sgRNA were delivered to postnatal day 4 (P4) Δ52;h53KI mice through intraperitoneal (IP) injection at a dose of 8×10¹³ vector genomes (vg)/kg for each AAV vector. Four weeks after systemic AAV delivery, the dystrophin protein expression was assessed in several muscle tissues, including tibialis anterior (TA) of the hindlimb, triceps of the forelimb, diaphragm, and cardiac muscle. Assessment by immunohistochemistry showed extensive dystrophin expression in skeletal muscles and the heart (FIG. 7).

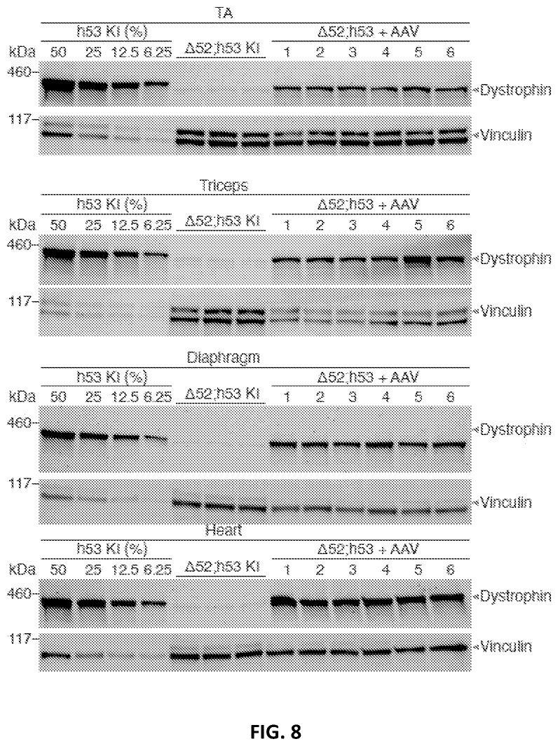

Western blot analysis of humanized DMD mice receiving systemic CRISPR/LRVQR-SpCas9 gene editing shows dystrophin restoration. To further quantitatively assess dystrophin protein restoration, Western blot analysis was performed in skeletal muscles and the heart of CRISPR/LRVQR-SpCas9 gene edited Δ52;h53KI DMD mice. Mice receiving the systemic AAV-CRISPR/LRVQR-SpCas9 treatment showed restored dystrophin protein expression in multiple skeletal muscles and heart (FIG. 8).

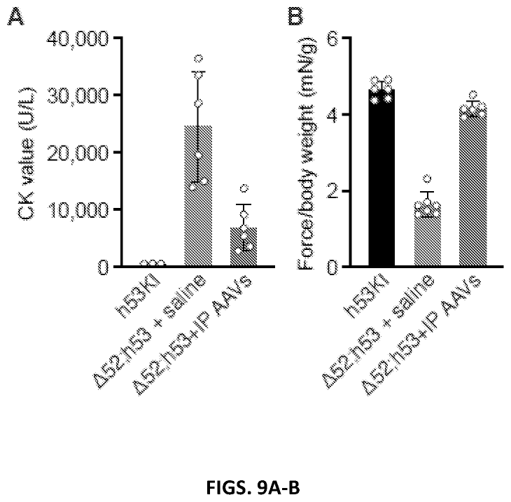

Systemic CRISPR/LRVQR-SpCas9 gene editing improves muscle function in humanized DMD mice. To examine the effect of dystrophin restoration on muscle integrity and function, serum CK levels and forelimb grip strength was measured in CRISPR/LRVQR-SpCas9 gene edited Δ52;h53KI DMD mice. Elevated serum CK is a pathological indicator of muscle damage. Compared to the h53KI control mice, the serum CK activity of Δ52;h53KI DMD mice injected with saline was elevated 52-fold. In contrast, the serum CK of CRISPR/LRVQR-SpCas9 gene edited Δ52;h53KI DMD mice was reduced by 72%, indicative of muscle protection from contraction-induced damage (FIG. 9A). Additionally, CRISPR/LRVQR-SpCas9 gene editing was shown to be able to improve muscle strength in Δ52;h53KI DMD mice. The grip strength of Δ52;h53KI DMD mice receiving saline was reduced by 65% compared to h53KI control mice (FIG. 9B). Δ52;h53KI DMD mice receiving systemic AAV-LRVQR-SpCas9 gene editing showed a significant improvement of forelimb grip strength to 89% of h53KI mice (FIG. 9B).

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Benvenisty and Neshif, Proc. Natl. Acad. Sci. USA, 83: 9551-9555, 1986.
Bushby et al., Lancet Neurol., 9(1): 77-93 (2010).
Bushby et al., Lancet Neurol., 9(2): 177-198 (2010).
Dubensky et al., Proc. Natl. Acad. Sci. USA, 81:7529-7533, 1984.
Hersdorffer et al., DNA Cell Biol., 9:713-723, 1990.
Markowitz et al., J. Virol., 62: 1120-1124, 1988.
Roux et al., Proc. Natl. Acad. Sci. USA, 86:9079-9083, 1989.
Walmsley et al., PLoS ONE 5, e8647, 2010.
Wang et al., Cell, 153: 910-910, 2013.
Himeda et al., Methods Mol Biol 709: 3-19, 2011.
Min et al., Mol Ther 28: 2044-2055, 2020.
Zhang et al., Sci Adv 6, eaay6812, 2020.
Hakim et al., JCI Insight 3.10.1172, 2018.
Min et al., Sci Adv 5, eaav4324, 2019.
Zhang et al., Sci Adv 3, e1602814, 2017.

1. A composition comprising:
(a) a first vector comprising a nucleic acid comprising:
a sequence encoding a first DMD guide RNA targeting a first genomic target sequence;
a sequence encoding a first promoter, wherein the first promoter drives expression of the sequence encoding the first DMD guide RNA, and
(b) a second vector comprising a nucleic acid comprising:
a sequence encoding an SpCas9 or a nuclease domain thereof, wherein said SpCas9 or nuclease domain thereof comprises Leucine or Valine at residue 1135, Arginine, Lysine or Serine at residue 1218, Valine, Glutamine or Phenylalanine at residue 1219, Glutamine or Valine at residue 1335 and Arginine at residue 1337 corresponding to the sequence shown in SEQ ID NO: 2;
a sequence encoding a muscle-specific promoter, wherein the muscle-specific promoter drives expression of the sequence encoding said SpCas9 or a nuclease domain thereof.

2. The composition of claim 1, wherein the ratio of the first vector to the second vector is 1:1 to 1:30 or from 1:1 to 30:1.

3. The composition of claim 1, wherein the ratio of the first vector to the second vector is 1:2, 1:3, 1:4, 1:5, 1:6, 1:7, 1:8, 1:9, 1:10, 1:15, 1:20 or 1:25.

4. The composition of claim 1, wherein the ratio of the second vector to the first vector is 1:2, 1:3, 1:4, 1:5, 1:6, 1:7, 1:8, 1:9, 1:10, 1:15, 1:20 or 1:25.

5. The composition of claim 1, wherein the first vector and/or the second vector comprises a sequence isolated or derived from an adeno-associated virus (AAV).

6. The composition of claim 1, wherein the sequence encoding the muscle-specific promoter comprises or consists of a sequence encoding a CK8 promoter, such as a CK8e promoter.

7. The composition of claim 1, wherein the DMD guide RNA comprises or consists of the sequence 5′-CUGUUGCCUCCGGUUCUGAA-3′ (SEQ ID NO: 1).

8. The composition of claim 1, wherein the composition comprises between 5×10¹¹ viral genomes (vg)/kilogram (kg) and 1×10¹⁵ vg/kg, inclusive of the endpoints, of the first vector, such as at least 5×10¹² viral genomes (vg)/kilogram (kg), at least 1×10¹³ viral genomes (vg)/kilogram (kg), at least 5×10¹³ viral genomes (vg)/kilogram (kg), at least 1×10¹⁴ viral genomes (vg)/kilogram (kg), at least 5×10¹⁴ viral genomes (vg)/kilogram (kg) of the first vector, or at least 1×10¹⁵ viral genomes (vg)/kilogram (kg) of the first vector.

9. The composition of claim 1, wherein the Cas9 nuclease or nuclease domain thereof has the mutations D1135L, G1218R, E1219V, R1335Q, and T1337R.

10. The composition of claim 1, further comprising a pharmaceutically-acceptable carrier.

11. A method of treating muscular dystrophy in a subject in need thereof comprising administering to the subject a therapeutically effective amount of the composition of claim 1.

12. The method of claim 11, wherein the composition is administered locally.

13. The method of claim 11, wherein the composition is administered directly to a muscle tissue.

14. The method of claim 11, wherein the composition is administered by an intramuscular infusion or injection.

15. The method of claim 11, wherein the muscle tissue comprises a tibialis anterior tissue, a quadriceps tissue, a soleus tissue, a diaphragm tissue, or a heart tissue.

16. The method of claim 11, wherein the composition is administered by intra-cardiac injection.

17. The method of claim 16, wherein the composition is administered systemically.

18. The method of claim 17, wherein the composition is administered by an intravenous infusion or injection.

19. The method of claim 11, wherein the subject is a neonate, an infant, a child, a young adult, or an adult.

20. The method of claim 11, wherein the subject has muscular dystrophy.

21. The method of claim 11, wherein the subject is a genetic carrier for muscular dystrophy.

22. The method of claim 11, wherein the subject is male.

23. The method of claim 11, wherein the subject is female.

24. The method of claim 11, wherein the subject is an adult.

25. The method of claim 24, wherein the adult is at least 18 years old or at least 25 years old.

26. The method of claim 24, wherein the adult is at least 20 kg.

27. The method of claim 11, wherein the subject is a child.

28. The method of claim 27, wherein the child is less than 18 years of age.

29. The method of claim 27, wherein the child is 20 kg or less.

30. The method of claim 11, wherein the subject is an infant.

31. The method of claim 30, wherein the subject is less than 2 years old.

32. The method of claim 11, wherein upon administering the therapeutically effective amount of the composition, the subject produces a minimal immune response to the composition.

33. The method of claim 32, wherein the minimal immune response to the composition is reduced or eliminated treatment with an anti-inflammatory agent or an immune suppressive agent.

34. The method of claim 11, wherein the composition does not induce breaks in a predicted alternative targeting site.

35. The method of claim 34, wherein the predicted alternative targeting site comprises a coding sequence of the human genome and wherein the coding sequence comprises at least 2 mismatches with respect to the first genomic target sequence.

36. The method of claim 35, wherein the predicted alternative targeting site comprises a coding sequence of the human genome and wherein the coding sequence comprises at least 3, 4, 5, 6, 7, 8, 9, or 10 mismatches with respect to the first genomic target sequence.

37. The method of claim 11, wherein the administration of the therapeutically effective amount of the composition is provided as a single dose or provided within a single medical procedure.

38. The method of claim 11, wherein the administration of the therapeutically effective amount of the composition is provided as multiple doses or provided over multiple medical procedures.

39. A Cas9 nuclease or nuclease domain comprising Leucine or Valine at residue 1135, Arginine, Lysine or Serine at residue 1218, Valine, Glutamine or Phenylalanine at residue 1219, Glutamine or Valine at residue 1335 and/or Arginine at residue 1337 corresponding to the sequence of SEQ ID NO: 2.

40. The Cas9 nuclease or nuclease domain of claim 39, wherein the Cas9 nuclease or nuclease domain comprises Leucine or Valine at residue 1135 corresponding to the sequence of SEQ ID NO: 2.

41. The Cas9 nuclease or nuclease domain of claim 39, wherein the Cas9 nuclease or nuclease domain comprises Arginine, Lysine or Serine at residue 1218 corresponding to the sequence of SEQ ID NO: 2.

42. The Cas9 nuclease or nuclease domain of claim 39, wherein the Cas9 nuclease or nuclease domain comprises Valine, Glutamine or Phenylalanine at residue 1219 corresponding to the sequence of SEQ ID NO: 2.

43. The Cas9 nuclease or nuclease domain of claim 39, wherein the Cas9 nuclease or nuclease domain comprises Glutamine or Valine at residue 1335 corresponding to the sequence of SEQ ID NO: 2.

44. The Cas9 nuclease or nuclease domain of claim 39, wherein the Cas9 nuclease or nuclease domain comprises Arginine at residue 1337 corresponding to the sequence of SEQ ID NO: 2.

45. The Cas9 nuclease or nuclease domain of claim 39, wherein the Cas9 nuclease has the mutations D1135L, G1218R, E1219V, R1335Q, and/or T1337R.

46. The Cas9 nuclease or nuclease domain of claim 39, wherein the Cas9 nuclease comprises a sequence that is at least 85%, 90%, 91%, 92%, 93%, 94%, 95%, 96%, 97%, 98%, 99%, or 100% identical the sequence of SEQ ID NO: 6, or biologically active fragments thereof.

47. The Cas9 nuclease or nuclease domain of claim 39, wherein the Ca9 nuclease or nuclease domain is derived from Staphylococcus pyogenes.

48. A nucleic acid encoding the Cas9 nuclease or nuclease domain of claim 39.

49. A nucleic acid encoding a Cas9 nuclease or nuclease domain comprising Leucine or Valine at residue 1135, Arginine, Lysine or Serine at residue 1218, Valine, Glutamine or Phenylalanine at residue 1219, Glutamine or Valine at residue 1335 and/or Arginine at residue 1337 corresponding to the sequence of SEQ ID NO: 2.

50. The nucleic acid of claim 49, wherein the Cas9 nuclease has the mutations D1135L, G1218R, E1219V, R1335Q, and T1337R.

51. The nucleic acid of claim 49, wherein the Cas9 nuclease comprises the sequence of SEQ ID NO: 6.

52. The nucleic acid of claim 49, wherein the Ca9 nuclease or nuclease domain is derived from Staphylococcus pyogenes.

53. The nucleic acid of claim 49, wherein the nucleic acid is an RNA.

54. The nucleic acid of claim 49, wherein the nucleic acid is a DNA.

55. The nucleic acid of claim 49, wherein the nucleic acid is located in a vector under the control of a promoter active in eukaryotic cells.

56. The nucleic acid of claim 55, wherein the vector is an AAV vector.

57. The nucleic acid of claim 55, wherein the promoter is a muscle-specific promoter, such as a CK8/CK8e promoter.

58. An sgRNA comprising the sequence 5′-CUGUUGCCUCCGGUUCUGAA-3′(SEQ ID NO: 1).

59. A nucleic acid encoding the sgRNA of claim 58.

60. The sgRNA of claim 58 or the nucleic acid of claim 59, wherein the sgRNA or nucleic acid is located in a vector under the control of a promoter active in eukaryotic cells.

61. The nucleic acid of claim 60, wherein the vector is an AAV vector.

62. A host cell comprising the nucleic acid of claim 48.
